(19) United States
(12) Patent Application Publication
(10) Pub. No.: US 2020/0179528 A9
(43) Pub. Date: Jun. 11, 2020

(54) IMMUNE CELL-TARGETED PARTICLES

(71) Applicants: Dana-Farber Cancer Institute, Inc., Boston, MA (US); Massachusetts Institute of Technology, Cambridge, MA (US)

(72) Inventors: Michael Solomon Goldberg, Brookline, MA (US); Daniela Schmid, Lenggries (DE); Darrell J. Irvine, Arlington, MA (US); Kai Wucherpfennig, Brookline, MA (US)

(73) Assignees: Dana-Farber Cancer Institute, Inc., Boston, MA (US); Massachusetts Institute of Technology, Cambridge, MA (US)

(21) Appl. No.: 16/065,489

(22) PCT Filed: Dec. 23, 2016
(86) PCT No.: PCT/US16/68541

§ 371 Date: Jun. 22, 2018

Related U.S. Application Data
(60) Provisional application No. 62/286,283, filed on Jan. 22, 2016.
(60) Provisional application No. 62/387,251, filed on Dec. 23, 2015.

(51) Int. Cl. A61K 47/68; A61K 47/69; C07K 16/28

(52) U.S. Cl. CPC A61K 47/6849; A61K 47/6937; C07K 2317/54; C07K 16/2803; A61K 47/6801

(57) ABSTRACT

The present disclosure provides particles with a polymeric core containing a pharmaceutically active agent; and an antibody fragment conjugated to the surface of the particle, wherein the antibody fragment targets an endogenous immune cell subset (e.g., an endogenous T-cell or a myeloid-derived suppressor cell). The present invention provides methods for forming and methods for using the particles. The particles described herein may be useful in treating and/or preventing proliferative disease, inflammatory disease, or neoplastic disorders (e.g., cancer, autoimmune diseases). Also provided in the present disclosure are pharmaceutical compositions, kits, methods, and uses including or using a particle described herein.

### RELATED APPLICATIONS

This application claims priority under 35 U.S.C. § 119(e) to U.S. Provisional Application, U.S. Ser. No. 62/387,251, filed Dec. 23, 2015, and U.S. Provisional Application, U.S. Ser. No. 62/286,283, filed Jan. 22, 2016, which are incorporated herein by reference.

### FIELD OF THE INVENTION

The present invention relates to a particle with a polymeric core containing a pharmaceutically active agent, and an antibody or fragment thereof conjugated to the surface of the particle, wherein the antibody or fragment thereof targets a T-cell; compositions including such particles, methods for preparing such particles, and uses of the particles for the treatment and prevention of disease. The present invention relates to a particle with a polymeric core containing a pharmaceutically active agent, and an antibody or fragment thereof conjugated to the surface of the particle, wherein the antibody or fragment thereof targets an endogenous immune cell subset (e.g., a T-cell, or myeloid-derived suppressor cell); compositions including such particles, methods for preparing such particles, and uses of the particles for the treatment and prevention of disease.

### BACKGROUND OF THE INVENTION

Particles are often used as delivery systems for pharmaceutically active agents. The use of nanoparticles allows the pharmaceutically active agent to be transported to and/or accumulate at a target site (e.g., the place of action), thereby minimizing undesirable side effects and lowering the required therapeutic dose. Moreover, encapsulation of pharmaceutically active agents in particles greatly enhances the therapeutic window of many pharmaceutically active agents, thereby reducing the frequency of administration. Many applications require the particles to be stable under physiological conditions, exhibit sustained or controlled release kinetics, and/or exhibit high loading capacity of the pharmaceutically active agent (e.g., drug).

Clinical data reveal that arousal of a patient's dormant immune system can produce durable benefit. (1). Challengingly, the proportion of patients who respond to cancer immunotherapy remains modest (<20%), and systemic immune stimulation is often associated with autoimmune-type pathologies, such as colitis and pneumonitis (2,3). The ability to concentrate the action of immunostimulatory drugs on tumor-reactive effector cells would improve both efficacy and safety, preventing stimulation of both immunosuppressive cells and non-tumor-reactive effector cells. To this end, nanoparticles that can target the delivery of immunotherapies to specific subsets of endogenous immune cells have been developed. Following intravenous administration, these particles bind to T cells in the circulation, which actively migrate to solid tumors, and can carry the particles into the harsh, immunosuppressive tumor microenvironment.

TGFβ is a major mediator of immunosuppression (4), but systemic administration of TGFβR1 inhibitors can be toxic owing to the importance of this signaling pathway in disparate cellular contexts (5). It was hypothesized that release of SD-208, a TGFβR1 inhibitor, in an autocrine- and/or paracrine-like manner would restore effector T cell function and thereby enable robust killing of cancer cells. Notably, the antibody fragments used to target the nanoparticles can also be used to impart immune checkpoint blockade, thereby further augmenting the functionality of exhausted T cells, such as those expressing PD-1.

The particles described herein increase the proportion of patients who respond to immunotherapy and to minimize the side effects that they experience. These particles have strong potential for clinical translation as they are prepared from the FDA-approved polymers poly(lactic-co-glycolic acid) (PLGA) and polyethylene glycol (PEG). PLGA/PEG-based nanoparticles have previously been used to target the delivery of cytotoxic chemotherapy (6) or molecular targeted therapy (7) to cancer cells based on binding to receptors expressed on the surface of the cancer cells.

Unfortunately, directly targeting receptors on the surface of cancer cells may not work, as targeted and untargeted particles exhibit similar biodistribution and tumor localization patterns (8). Most nanoparticles rely on passive accumulation into tumors, and their efficacy has been most pronounced in preclinical models of solid tumors that harbor leaky vasculature (9), which may not reflect tumors that grow over the course of years rather than days. In contrast, immune cells traffic actively down chemokine gradients to sites of inflammation, such as tumors. Indeed, leveraging T cells as vectors greatly enhances the quantity of drug that can be delivered to tumors, achieving levels in the tumor that are orders of magnitude greater than that which can be delivered by nanoparticles alone (10). Furthermore, most approaches to date have focused on the delivery of cytotoxic agents, which must kill the vast majority of the target cells in order to be effective. Much lower concentrations of immunomodulatory drugs are required, as such compounds can stimulate an amplifying response.

The conjugation of drug-containing liposomes to the surface of T cells prior to adoptive cell transfer dramatically improves the potency of the administered cells (11, 12). The liposomes, however, become diluted as the cells proliferate. It was next shown that adoptively transferred T cells can be effectively targeted in vivo with surface-modified liposomes, enabling repeated expansion of the transferred cells (13). The targeting of endogenous immune cells in the absence of the cumbersome and costly procedures associates with adoptive cell transfer was sought. The delivery of a small molecule immunomodulator in a targeted manner via these nanoparticles was also sought.

It was hypothesized that delivery of immunomodulatory compounds via T cell-targeting nanoparticles would augment T cell function better than systemic administration of free drug. To this end, it has been shown that the T cell-targeting particles can be targeted to particular endogenous T cell subsets in blood, secondary lymphoid organs, and tumors. Importantly, the particles can be targeted to surface receptors in a modular manner, as we have confirmed targeting of lineage markers (e.g., CD8, Gr-1) as well as functional markers (e.g., PD-1, GITR). This modularity extends to the entrapped payload, as the particles can be loaded with a variety of small molecule drugs, which are released from the particles in a sustained manner. We show specific binding in vitro and in vivo. Targeted delivery of an inhibitor of TGFβ signaling to PD-1-expressing T cells delays tumor growth and extends the survival of mice harboring colorectal tumors relative to administration of free drug. Excitingly, targeted delivery of a TLR7/8 to PD-1-expressing T cells can inflame a non-inflamed tumor, providing a novel approach to improving the percentage of patients who respond to cancer immunotherapy. Accordingly, improved particles, compositions of such particles, and methods for preparing and using such particles for targeted drug delivery are needed.

### SUMMARY OF THE INVENTION

The present invention provides particles that target T-cells, in particular endogenous T-cells, compositions thereof, formulations, and kits useful for administration of the particles to a subject. The present invention also provides methods of preparing such particles. The present invention provides a method of treating a proliferative disease in a subject comprising administering the particles or compositions thereof to a subject in need of treatment for a proliferative disease.

In one aspect, a nanoparticle comprising a polymeric core containing at least one pharmaceutically active agent and an antibody or fragment thereof conjugated to the surface of the particle, wherein the antibody or fragment thereof targets a T-cell, is provided. In one aspect, a particle comprising a polymeric core containing at least one pharmaceutically active agent and an antibody fragment conjugated to the surface of the particle, wherein the antibody fragment targets an endogenous immune cell subset, is provided. In some embodiments, the endogenous immune cell subset is a T-cell. In some embodiments, the endogenous immune cell subset is a myeloid-derived suppressor cell. In some embodiments, the particle is not an artificial antigen presenting cell. In some embodiments, the particles are not artificial antigen presenting cells. In some embodiments, the nanoparticles are not artificial antigen presenting cells. In some embodiments, the antibody or fragment thereof is an antibody fragment. In some embodiments, the antibody fragment is enzymatically produced by fragmentation of an intact antibody using IdeS or IdeZ. In some embodiments, the antibody fragment is enzymatically produced by fragmentation of an intact antibody using IdeS or IdeZ has a defined sequence. In some embodiments, the antibody or fragment thereof is directly conjugated to the surface of the particle. In some embodiments, the antibody fragment is directly conjugated to the surface of the particle. In some embodiments, the antibody fragment is derived from nivolumab, pembrolizumab, PDR001, MBG453, LAG525, or GWN323. In some embodiments, the antibody or fragment thereof targets GITR or Gr-1. In some embodiments, the antibody or fragment thereof targets PD-1 or GITR, which are expressed on the surface of T-cells. In some embodiments, the antibody or fragment thereof targets Gr-1, which is expressed on the surface of myeloid-derived suppressor cells. Gr-1, or its human equivalent, may include but is not limited to CCR2, CD11b, CD14, CD15, CD33, CD39, CD66b, CD124, IL4Ra, and/or S100 family members, including S100A8, S100A9, S10A12. In certain embodiments, an antibody or fragment thereof targeting two of these receptors is used. In some embodiments, the particle comprises a corona around at least a portion of the surface of the particle core. In some embodiments, the corona comprises a polymer. In some embodiments, the corona comprises polyethylene glycol (PEG). In some embodiments, the corona has a moiety allowing for attachment of the antibody fragment to the surface of the particle. In some embodiments, the PEG corona has a moiety allowing for attachment of the antibody fragment to the surface of the particle. In certain embodiments, the moiety is an electrophile-PEG corona. In certain embodiments, the electrophile-PEG corona is a maleimide-PEG corona. In certain embodiments, the maleimide-PEG corona allows for attachment of the antibody fragment to the surface of the particle. In some embodiments, the particle comprises a polyethylene glycol (PEG) coating covering the surface of the particle core. In some embodiments, the PEG coating has a maleimide-PEG corona moiety allowing for attachment of the antibody or fragment thereof to the surface of the particle. In some embodiments, the antibody or fragment thereof is directly conjugated to the PEG-PLGA nanoparticle. In some embodiments, the antibody or fragment thereof is not non-covalently bound (e.g., biotin/streptavidin) to the surface of the particle. In some embodiments, the antibody or fragment thereof is covalently bound to the surface of the particle. In some embodiments, the antibody or fragment thereof is not non-covalently bound to the PEG-PLGA nanoparticle. In some embodiments, the antibody or fragment thereof is covalently bound to the PEG-PLGA nanoparticle. The antibody or fragment thereof attached to the particle targets particular T-cells, allowing the delivery of the pharmaceutically active agent within the particle to particular T-cells. In certain embodiments, the antibody or fragment thereof attached to the particle targets particular T-cells, allowing the delivery of the pharmaceutically active agent within the particle to particular T-cells or to tissues in which such T cells reside or to tissues to which such T-cells migrate. In some embodiments, the antibody or fragment thereof targets a CD4+ T-cell. In some embodiments, the antibody or fragment thereof targets an effector T-cell. In some embodiments, the antibody fragment targets an effector T-cell in vivo. In some embodiments, the antibody or fragment thereof targets a regulatory T-cell. In some embodiments, the antibody fragment targets a regulatory T-cell in vivo. In some embodiments, the antibody or fragment thereof targets a suppressor cell. In some embodiments, the antibody or fragment thereof targets a myeloid-derived suppressor cell. In some embodiments, the antibody fragment targets a myeloid-derived suppressor cell. In some embodiments, the antibody or fragment thereof targets a myeloid-derived suppressor cell (MDSC) in vivo. In some embodiments, the target of the antibody fragment is Gr-1. In certain embodiments, the particle is internalized by T-cells (e.g., activated T-cells, activated CD8+ T-cells). In some embodiments, endogenous T-cells are targeted. In some embodiments, activated T-cells (e.g., activated CD8+ T-cells) are targeted. In some embodiments, the target of the antibody or fragment thereof is selected from the group consisting of PD-1, Thy1.1, CD8, CD137, LAG-3, and TIM-3. In some embodiments, the target of the antibody fragment is selected from the group consisting of PD-1, CD8, CD25, CD27, LAG-3, TIM-3, BTLA, VISTA, TIGIT, NRP1, TNFRSF25, OX40, GITR, and ICOS. In some embodiments, the T-cell is a CD8+ T-cell. In some embodiments, the T-cell is a CD4+ T-cell.

In some embodiments, the particle comprises a biodegradable polymer, and has a high encapsulation efficiency of the pharmaceutically active agent. In some embodiments, the biodegradable polymer has a sustained release of the pharmaceutically active agent. In some embodiments, the pharmaceutically active agent is an immunomodulatory compound. In certain embodiments, the pharmaceutically active agent is an inhibitor of TGFβ signaling. In certain embodiments, the pharmaceutically active agent is an inhibitor of the TGFβ receptor I kinase. In certain embodiments, the pharmaceutically active agent binds to the TGFβ receptor I kinase. In certain embodiments, the pharmaceutically active agent specifically binds to the TGFβ receptor I kinase. In certain embodiments, the pharmaceutically active agent is compound SD-208. In certain embodiments, the pharmaceutically active agent is a toll-like receptor (TLR) agonist. In certain embodiments, the pharmaceutically active agent is a TLR7 agonist. In certain embodiments, the pharmaceutically active agent is a TLR8 agonist. In certain embodiments, the pharmaceutically active agent is an agonist of TLR7 and TLR8. In certain embodiments, the pharmaceutically active agent is resiquimod (R848). In certain embodiments, the pharmaceutically active agent increases the proportion of CD8+ T cells in the tumor. In certain embodiments, the pharmaceutically active agent increases the proportion of granzyme B-expressing CD8+ T cells in the tumor. In certain embodiments, the pharmaceutically active agent increases the proportion of IFNγ-expressing CD8+ T cells in the tumor. In certain embodiments, targeted delivery of a TLR agonist to PD-1+ T cells inflames a non-T-cell-inflamed tumor, which improves patient responses to cancer immunotherapy.

In some embodiments, the polymeric core contains two or more agents to be delivered. In another aspect, methods of forming the particle are provided. In another aspect, methods of using the particle are provided. In some embodiments, the method includes providing a polymeric core containing a pharmaceutically active agent; and conjugating an antibody or fragment thereof to the surface of the particle, wherein the antibody or fragment thereof targets a T-cell. In some embodiments, the method includes providing a polymeric core containing a pharmaceutically active agent; and conjugating an antibody fragment to the surface of the particle, wherein the antibody fragment targets an endogenous immune cell subset. In some embodiments, the endogenous immune cell subset is a T-cell. In some embodiments, the endogenous immune cell subset is a myeloid-derived suppressor cell. In some embodiments, the method includes targeting a T-cell to deliver pharmaceutical agents to specific T-cells for the treatment of proliferative disease. In some embodiments, the method includes targeting an endogenous immune cell subset to deliver pharmaceutical agents to cells in the tumor microenvironment or draining lymph node for the treatment of proliferative disease. In another aspect, the present invention provides methods of using the T-cell targeted particle for the treatment of proliferative disease. In another aspect, the present invention provides methods of using the endogenous immune cell subset-targeted particle for the treatment of proliferative disease. In another aspect, the present invention provides use of the particle for the treatment of proliferative disease. In some embodiments, the proliferative disease is cancer. In some embodiments, the cancer is colorectal cancer. In some embodiments, the cancer is metastatic colorectal cancer. In some embodiments, the cancer is melanoma. In some embodiments, the cancer is metastatic melanoma. In some embodiments, the proliferative disease is autoimmune disease. In some embodiments, the proliferative disease is inflammatory disease. In some embodiments, the proliferative disease is neoplastic disorder.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### Definitions

“Antibody”: The term “antibody” refers to an immunoglobulin, whether natural or wholly or partially synthetically produced. All derivatives or fragments thereof which maintain specific binding ability are also included in the term. The term also covers any protein having a binding domain which is homologous or largely homologous to an immunoglobulin binding domain. An antibody may be monoclonal or polyclonal. The antibody may be a member of any immunoglobulin class, including any of the human classes: IgG, IgM, IgA, IgD, and IgE. In certain embodiments, antibodies of the IgG class are used.

“Antibody fragment”: The term “antibody fragment” refers to any derivative of an antibody which is less than full-length. Examples of antibody fragments include, but are not limited to, Fab, Fab′, F(ab′)₂, scFv, Fv, dsFv diabody, Fc, and Fd fragments. In certain embodiments, the fragment is an Fab fragment, more particularly an F(ab′)₂ fragment of an IgG antibody. In certain embodiments, the fragment is a F(ab′)₂ fragment. In certain embodiments, the fragment is a Fab fragment. In certain embodiments, the fragment is a Fab′ fragment. The antibody fragment may be produced by any means. For instance, the antibody fragment may be enzymatically or chemically produced by fragmentation of an intact antibody, or it may be recombinantly produced from a gene encoding a partial antibody sequence. Alternatively, the antibody fragment may be wholly or partially synthetically produced. The antibody fragment may be a single chain antibody fragment. A functional antibody fragment will typically comprise at least about 50 amino acids and more typically will comprise at least about 200 amino acids. In some embodiments, the antibody fragment may be enzymatically produced by fragmentation of an intact antibody using IdeS or IdeZ.

“Administer”: The terms “administer,” “administering,” or “administration,” as used herein, refers to implanting, absorbing, ingesting, injecting, inhaling, or otherwise introducing an inventive particle, or a composition thereof, in or on a subject.

“Biocompatible”: As used herein, the term “biocompatible” is intended to describe a material (e.g., particles, excipients) that is not toxic to cells. Particles are “biocompatible” if their addition to cells in vitro results in less than 20% (e.g., less than 15%, less than 10%, less than 5%, less than 3%, less than 2%, less than 1%) cell death, and their administration in vivo does not induce inflammation or other such adverse effects.

“Biodegradable”: As used herein, “biodegradable” compounds or materials are those that, when introduced into cells, are broken down by the cellular machinery or by hydrolysis into components that the cells can either reuse or dispose of without significant toxic effects on the cells (i.e., fewer than about 20% of the cells are killed when the components are added to cells in vitro). The components preferably do not induce inflammation or other adverse effects in vivo. In certain embodiments, the chemical reactions relied upon to break down the biodegradable compounds are not catalyzed. For example, the inventive materials may be broken down in part by the hydrolysis of the polymeric material of the inventive particles.

“Biological macromolecule”: The term biological macromolecule refers to a macromolecule comprising at least 10 (e.g., at least 15, at least 25, at least 50) sugar, amino acid, and/or nucleotide repeating units. The biological molecule may be capable of undergoing a biological binding event (e.g., between complementary pairs of biological molecules) with another biological molecule. The biological macromolecule may be a nucleic acid, protein, peptide, or carbohydrate.

“Composition”: The terms “composition” and “formulation” are used interchangeably.

“Condition”: As used herein, the terms “condition,” “disease,” and “disorder” are used interchangeably.

“Particle”: As used herein, the term “particle” refers to a small object, fragment, or piece of material and includes, without limitation, microparticles and nanoparticles. Particles may be composed of a single substance or multiple substances. In certain embodiments, the particles are substantially solid throughout and/or comprise a core that is substantially solid throughout. In some embodiments, a particle may not include a micelle, a liposome, or an emulsion. The term “nanoparticle” or “NP” refers to a particle having a characteristic dimension (e.g., greatest dimension, average diameter) of less than about 1 micrometer and at least about 1 nanometer, where the characteristic dimension of the particle is the largest cross-sectional dimension of the particle. The term “microparticle” refers to a particle having a characteristic dimension of less than about 1 millimeter and at least about 1 micrometer, where the characteristic dimension of the particle is the smallest cross-sectional dimension of the particle. In certain embodiments, the particle is not an artificial antigen presenting cell.

“Pharmaceutically active agent”: As used herein, the term “pharmaceutically active agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Pharmaceutically active agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. Preferably, though not necessarily, the pharmaceutically active agent is one that has already been deemed safe and effective for use in humans or animals by the appropriate governmental agency or regulatory body. For example, drugs approved for human use are listed by the FDA under 21 C.F.R. § § 330.5, 331 through 361, and 440 through 460, incorporated herein by reference; drugs for veterinary use are listed by the FDA under 21 C.F.R. § § 500 through 589, incorporated herein by reference. All listed drugs are considered acceptable for use in accordance with the present invention. In certain embodiments, the pharmaceutically active agent is a small molecule. In certain embodiments, the pharmaceutically active agent is a biologic. In certain embodiments, the pharmaceutically active agent is not a biologic. In certain embodiments, the pharmaceutically active agent is not a protein. In certain embodiments, the pharmaceutically active agent is not a nucleic acid. In certain embodiments, the pharmaceutically active agent is not an anti-CD137 antibody. In certain embodiments, the pharmaceutically active agent is not interleukin-2 (IL-2). In certain embodiments, the pharmaceutically active agent is not IL-2-Fc fusion protein. In certain embodiments, the pharmaceutically active agent is not a vaccine. In certain embodiments, the pharmaceutically active agent is not a source of antigen for vaccination. Exemplary pharmaceutically active agents include, but are not limited to, anti-cancer agents, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, prostaglandins, immunomodulatory agents, etc.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to pharmaceutically active agent, whether naturally-occurring or artificially created (e.g., via chemical synthesis) that has a relatively low molecular weight. Typically, a small molecule is an organic compound (i.e., it contains carbon). The small molecule may contain multiple carbon-carbon bonds, stereocenters, and other functional groups (e.g., amines, hydroxyl, acyls, and heterocyclic rings, etc.). In certain embodiments, the molecular weight of a small molecule is at most about 2,500 g/mol, is at most about 2,000 g/mol, at most about 1,500 g/mol, at most about 1,250 g/mol, at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, at least about 900 g/mol, or at least about 1,000 g/mol. Combinations of the above ranges (e.g., at least about 200 g/mol and at most about 2,500 g/mol, at least about 200 g/mol and at most about 2,000 g/mol, at least about 200 g/mol and at most about 1,500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions.

“Solubility”: As used herein, “solubility” refers to the ability of a molecule to be carried in the solvent without precipitating out. The solubility may be expressed in terms of concentration of the saturated solution of the molecule at standard conditions.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female at any stage of development. The animal may be a transgenic animal or genetically engineered animal. In certain embodiments, the subject is non-human animal. In certain embodiments, the animal is fish. A “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

“Surface modifying agents”: As used herein, the term “surface modifying agent” refers to any chemical compound that can be attached to the surface of a particle. The surface modifying agent may be any type of chemical compound including small molecules, polynucleotides, proteins, peptides, metals, polymers, oligomers, organometallic complexes, lipids, carbohydrates, etc. The agent may modify any property of particle including surface charge, hydrophilicity, hydrophobicity, zeta potential, size, thickness of coating, etc. In certain embodiments, the surface modifying agent is a polymer such as polyethylene glycol (PEG) or co-polymers thereof.

As defined herein, the term “target tissue” refers to any biological tissue of a subject (including a group of cells, a body part, or an organ) or a part thereof, including blood and/or lymph vessels, which is the object to which a compound, particle, and/or composition of the invention is delivered. A target tissue may be an abnormal or unhealthy tissue, which may need to be treated. A target tissue may also be a normal or healthy tissue that is under a higher than normal risk of becoming abnormal or unhealthy, which may need to be prevented. The term “target cells” refers to a group of cells, or a part thereof, to which a compound, particle, and/or composition of the invention is delivered. Target cells may include cells in the immune response, for example, T-cells. “T-cells” are equivalent to “T cells.” A “non-target tissue” is any biological tissue of a subject (including a group or type of cells, a body part, or an organ) or a part thereof, including blood and/or lymph vessels, which is not a target tissue.

“Targeting moiety”: The term “targeting moiety” refers to a chemical moiety that facilitates localization to a particular targeting site, such as a tumor, a disease site, a tissue, an organ, a type of cell, or an organelle, and is able to bind to or otherwise associate with a biological moiety, for example, a membrane component, a cell surface receptor, organelle component, or the like. The targeting moiety may be directly bound to the particle or may be associated with the particle through a linking moiety. A variety of targeting moieties that direct pharmaceutical compositions to particular cells are known in the art (see, for example, Cotten et al., Methods Enzym., 217: 618, 1993; incorporated herein by reference). Classes of targeting moieties useful in the inventive particles include proteins, peptides, polynucleotides, small organic molecules, metals, metal complexes, carbohydrates, lipids, etc.

“Therapeutically effective amount”: As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent, alone or in combination with other therapies, which provides a therapeutic benefit in the treatment of the disease, disorder, or condition. The term “therapeutically effective amount” can encompass an amount that improves overall therapy, reduces or avoids symptoms or causes of disease or condition, or enhances the therapeutic efficacy of another therapeutic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

“Treatment”: As used herein, the terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

The term “inhibition”, “inhibiting”, “inhibit,” or “inhibitor” refer to the ability of a compound to reduce, slow, halt or prevent activity of a particular biological process in a cell relative to a vehicle.

The terms “condition,” “disease,” and “disorder” are used interchangeably.

The term “biologic” refers to large, complex molecules or mixtures of molecules produced in a living system (e.g., in a microorganism, plant, or animal cells). Examples of biologics include, but are not limited to vaccines, gene therapies, cellular therapies, antibodies (e.g., anti-CD137 antibodies), blood and blood components, tissues, nucleic acids, and proteins (e.g., cytokines (e.g., interleukin-2 (IL-2))).

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (e.g., “malignant neoplasms”), benign neoplasms, angiogenesis, inflammatory diseases, and autoimmune diseases.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1B are (A) a schematic of the in vitro characterization of the anti-CD8 nanoparticles (NP), including the size distribution of optimized blank NP's, anti-CD8 NP's, and control formulations, and the Polydispersity index (PDI) of each set of NP's; (B) confocal microscopy images of the CD8 and isotype NP's on the CD8+ T-cell surface.

FIG. 2 is a schematic of the activation of the ovalbumin-specific (OT-1) CD8+ T-cells by B16 tumor cells following CD8-NP binding.

FIG. 3 is a schematic of the binding of anti-CD8 NP's in vivo, in blood, inguinal lymph nodes (LN), and spleen.

FIG. 4 is a schematic of the binding of anti-CD8 NP's in tumor-bearing mice.

FIG. 5 is a schematic of a small molecule inhibitor (SMI) screen to assess the immunomodulatory effects of the SMI's.

FIG. 6 is a schematic of the internalization of CD8-targeted nanoparticles (NP) by CD8+ T-cells.

FIGS. 7A-7G show encapsulation and release of immunomodulatory compounds. FIG. 7A is the structure of SD-208, a TGF-βRI inhibitor (IC₅₀=49 nM); FIG. 7B is an absorbance scan of SD-208 dissolved in DMSO; absorbance maximum was identified at 370 nm; FIG. 7C is the standard curve used to measure percent drug encapsulation prepared in blank nanoparticle matrix at 370 nm; FIG. 7D is a scheme of the single-emulsion evaporation method that was used for drug encapsulation; FIG. 7E shows the entrapment efficiencies and size distributions of nanoparticles using different polymers (PDI: polydispersity index); FIG. 7F shows the release kinetics of SD-208 into PBS containing 10% FBS at 0.33 mg polymer/mL of release medium; FIG. 7G shows the encapsulation of other immunomodulatory compounds in maleimide AP41-based PEG-PLGA nanoparticles.

FIGS. 8A-8D show optimization of F(ab′)2 conjugation to polymer-based nanoparticles. FIG. 8A is the scheme of antibody conjugation to nanoparticle (NP) surface; FIG. 8B is a Coomassie-stained SDS gel (non-reducing conditions) after cleavage of anti-CD8a and isotype control antibody for 2 h with IdeS/FabRICATOR; FIG. 8C shows that shows that various amounts of DTT and maleimide-functionalized PEG-PLGA were evaluated to optimize F(ab′)2 fragment conjugation, as measured by BCA protein assay; the optimized formulation yielded 27.5±4.7% conjugation efficiency; FIG. 8D is a Western blot of an SDS gel (reducing conditions) of CD8a-targeting nanoparticles using Fab- or Fc-specific antibodies.

FIGS. 9A-9D show in vitro characterization of CD8-targeting nanoparticles. FIG. 9A shows the size distribution of optimized blank anti-CD8a NPs and control formulations (PDI: polydispersity index); FIG. 9B shows the binding of NPs (labeled with fluorescein) to the surface of CD8+ T cells isolated from a mouse spleen assessed by flow cytometry after 5 or 30 min; FIG. 9C shows the dose-dependent binding of DiD (dye)-labeled NPs to CD8+ T cells (Iso: isotype control antibody); FIG. 9D. is confocal microscopy after incubation of CD8+ T cells with NPs for 2 h; data analysis performed with ImageJ shows the NPs on the T cell surface.

FIG. 10 shows that T cells proliferate following activation by B16-Ova tumor cells, even when bound by nanoparticles. OT-I CD8+ T cells were incubated with anti-CD8a NPs (or relevant negative controls) for 30 min, washed to remove unbound NPs, and co-cultured with ovalbumin-expressing B16 tumor cells for 72 hours. Proliferation was assessed by CFSE dilution, and NP binding was assessed by fluorescence of DiD, which had been entrapped in the NP core.

FIG. 11 shows the binding of Thy1.1-targeted nanoparticles to the T cell surface. Fluorescein-labeled NPs targeting Thy1.1 were prepared as described in FIG. 8. T cells (CD4 or CD8) were incubated with NPs for 30 min, and the fluorescence intensity was assessed by flow cytometry.

FIG. 12 shows that the targeted nanoparticles bind to endogenous T cells in vivo. DiD-loaded CD8a-targeting NPs were injected intravenously and detected on T cells in the blood, inguinal lymph nodes (LN), and spleen after 2 h. The negative control (rat IgG2b isotype) is shown in red.

FIGS. 13A-13C show that T cell-targeting nanoparticles bind to endogenous T cells in tumor-bearing mice. FIG. 13A shows an experimental protocol: B16 melanoma cells were injected subcutaneously into C57BL/6 mice, which developed tumors over 13 days to a size of ˜400 mm³. 1 mg of nanoparticles was injected intravenously. Blood, tumors, tumor-draining lymph nodes, and spleens were collected 1, 24, or 48 h later. FIG. 13B shows the flow cytometry gating strategy for a tumor isolated after 24 h. FIG. 13C shows quantification of CD3/CD8-positive T cells in the left panel and DiD-positive CD3/CD8+ T cells in the right panel.

FIGS. 14A-14C show characterization of PD-1-targeting nanoparticles. FIG. 14A is a non-reducing SDS-PAGE stained with Coomassie Brilliant Blue following enzymatic cleavage of anti-PD-1 and mouse IgG2a isotype control antibodies using IdeZ; FIG. 14B is a Western blot after reducing SDS-PAGE of PD-1-targeting NPs developed with Fab-specific (left panel) or Fc-specific antibodies (right panel); lane 1: uncoated NPs, lane 2: isotype control NPs, lane 3: anti-PD-1 NPs, lane 4: anti-PD-1 F(ab′)2 and Fc cleavage products as positive control; FIG. 14C is a non-reducing SDS-PAGE stained with Coomassie Brilliant Blue following enzymatic cleavage of Pembrolizumab and human IgG4 isotype control into F(ab′)2 and Fc using IdeS.

FIGS. 15A-15B show that PD-1-targeting nanoparticles bind to T cells activated by cancer cells in vitro and to endogenous T cells in tumors in vivo. FIG. 15A shows CD8+ OT-I T cells that were activated with ovalbumin-expressing B16 melanoma cells (ratio 1:10 B16 to T cell) for 48 h and incubated with DiD-loaded, PD-1-targeting NPs for 30 min prior to DiD detected by flow cytometry. FIG. 15B shows C57BL/6 mice that were inoculated subcutaneously with ovalbumin-expressing B16 melanoma cells. NPs were injected intravenously when tumors grew to a size of ˜400 mm³. T cells in tumors were assessed for binding of PD-1-targeting NPs 1 h post-injection; quantification in panel at right.

FIG. 16 shows that PD-1-targeting nanoparticles bind to CD8+ T cells in the blood of tumor-bearing mice. C57BL/6 mice were inoculated subcutaneously with ovalbumin-expressing B16 melanoma cells. NPs were injected intravenously when tumors grew to a size of ˜400 mm³. T cells in the blood, spleen, and tumor-draining lymph node (TdLN) were assessed for binding of PD-1-targeting NPs 1 h post-injection; quantification in the right panels. Note that it may take longer than 1 h for NPs to be observed in the spleen and TdLN (and in higher proportions among T cells in the blood); indeed, there are very few PD-1+ T cells in the blood, spleen, and TdLN at this time point, but circulating T cells may enter these compartments given more time.

FIGS. 17A-17D show that PD-1-targeting nanoparticles bind to activated human T cells. FIG. 17A shows PD-1 expression on human CD3 T cells following activation with anti-CD3/CD28 complexes, n=4 independent donors±SEM; FIG. 17B shows dose-dependent binding of anti-PD-1 NPs to 250,000 activated human T cells is confirmed; negative control (hIgG4 isotype) shown in blue; FIG. 17C shows a quantification of T cells bound by DiD-loaded NPs; μg of NPs per 250,000 T cells (graph shows the results of two donors and is representative for at least two independent experiments); FIG. 17D shows the pre-incubation of activated (PD-1-expressing) T cells with free pembrolizumab (“pre pembro”) for 30 min, which blocks the binding of anti-PD-1 NPs (10 μg/200,000 T cells), n=3±SD.

FIGS. 18A-18D shows that delivery of TGFβR1 inhibitor (SD-208) from nanoparticles phenocopies free drug in vitro. FIG. 18A is the release profile of optimized NP formulation that was used for cellular assays (without DMSO as co-solvent in the organic phase); FIG. 18B shows the proliferation of CD8+ T cells following activation with anti-CD3/CD28 beads (1:2 bead to T cell ratio) for 72 hours in the presence or absence of TGFβ1 (2 ng/mL); quantification of geometric mean of cell trace violet (CTV), which is diluted upon proliferation, is shown in the right panel, n=3 ±SD; FIG. 18C shows intracellular granzyme B expression assessed by flow cytometry, n=3 ±SD; FIG. 18D shows interferon-γ (IFNγ) measured by ELISA, n=4±SEM.

FIG. 19 shows that targeted delivery of a TGFβR1 inhibitor (SD-208) to PD-1-expressing cells delays tumor growth, while free drugs and untargeted drug do not. 200,000 MC38 cells were injected subcutaneously in 100 μl PBS into C57BL/6 mice on day 0. Five days later, twice weekly treatment (administered intravenously) was initiated for a total of up to seven doses. 1) no treatment, 2) anti-PD-1 IgG+free SD-208, 3) untargeted empty particles, 4) untargeted particles loaded with SD-208, 5) PD-1-targeting empty particles, 6) PD-1-targeting empty particles+free SD-208, 7) PD-1-targeting particles loaded with SD-208. The dose was 20 μg for anti-PD-1 and 40 μg for SD-208. Note that an antitumor effect is observed only when the small molecule is delivered via the targeted particles. Iso, isotype control.

FIG. 20 shows that targeted delivery of a TGFβR1 inhibitor (SD-208) to PD-1-expressing cells extends survival of tumor-bearing mice, while free drugs and untargeted drug do not. 200,000 MC38 cells were injected subcutaneously in 100 μl PBS into C57BL/6 mice on day 0. Five days later, twice weekly treatment (administered intravenously) was initiated for a total of up to seven doses. 1) no treatment, 2) anti-PD-1 IgG+free SD-208, 3) untargeted empty particles, 4) untargeted particles loaded with SD-208, 5) PD-1-targeting empty particles, 6) PD-1-targeting empty particles+free SD-208, 7) PD-1-targeting particles loaded with SD-208. The dose was 20 μg for anti-PD-1 and 40 ug for SD-208. Note that an antitumor effect is observed only when the small molecule is delivered via the targeted particles. Iso, isotype control.

FIG. 21 shows that various small molecules can be efficiently loaded into the nanoparticles, which sustain the release of the payloads. FIG. 21A shows data for an inhibitor of IDO (epacadostat, INCB024360). FIG. 21B shows data for an agonist of TLR7/8 (resiquimod, R848). FIG. 21C shows data for an inhibitor of JAK (ruxolitinib). The formulation procedure used is the same as that used in FIG. 1. Release was measured by absorbance maximum at 280 nm, 300 nm, and 340 nm, respectively. 22.5 mg of PLGA and 7.5 mg of PLGA-PEG were used along with 3 mg (10%), 6 mg (20%), or 12 mg (40%) of small molecule. Note that the release profile can be delayed by loading less drug. Note that the encapsulation efficiency can increase (epacadostat) or decrease (resiquimod, ruxolitinib) by increasing initial loading amount.

FIG. 22 shows that T cell-targeting nanoparticles can be internalized. F(ab′)2-conjugated nanoparticles were loaded with DiD and labeled using the pHAb Amine Reactive Dye (G9841, Promega). This dye emits minimal fluorescence when situated in environment of pH greater 7 but fluoresces at 532/560 nm in acidic solution (as found in lysosomal cell compartments). CD8+ T cells were incubated with CD8-targeting nanoparticles for the indicated amount of time, and the fluorescent signal was measured by flow cytometry. DiD was used to confirm nanoparticle binding, and the fluorescence intensity of the pHAb dye was used as a measure of nanoparticle internalization. Such internalization depends on the receptor being targeted and was not observed for all targets.

FIG. 23 shows that the targeted delivery of a TLR7/8 agonist (R848) to PD-1-expressing cells increases the proportion of immune cells (CD45+) in MC38 tumors. 200,000 MC38 cells were injected subcutaneously in 100 μl PBS into C57BL/6 mice on day 0. Fourteen days later, a single intravenous injection was performed. Group 1 is PBS, Group 2 is free anti-PD-1 and free R848, Group 3 is free anti-PD-1 and R848 loaded in untargeted nanoparticles (isotype control), and Group 4 is R848 loaded in PD-1-targeted nanoparticles. The dose was 20 μg for anti-PD-1 and 60 μg for R848. After 72 hours, tumors were harvested, processed into single-cell suspensions, and analyzed by flow cytometry.

FIG. 24 shows that the targeted delivery of a TLR7/8 agonist (R848) to PD-1-expressing cells increases the proportion of Granzyme B- and IFNγ-positive CD8+ T cells in MC38 tumors. 200,000 MC38 cells were injected subcutaneously in 100 μl PBS into C57BL/6 mice on day 0. Fourteen days later, a single intravenous injection was performed. Group 1 is PBS, Group 2 is free anti-PD-1 and free R848, Group 3 is free anti-PD-1 and R848 loaded in untargeted nanoparticles (isotype control), and Group 4 is R848 loaded in PD-1-targeted nanoparticles. After 72 hours, tumors were harvested, processed into single-cell suspensions, and analyzed by flow cytometry.

FIG. 25 shows targeted delivery of a TLR7/8 agonist (R848) to PD-1-expressing cells promotes infiltration of CD8+ T cells into MC38 tumors. 200,000 MC38 cells were injected subcutaneously in 100 μl PBS into C57BL/6 mice on day 0. Fourteen days later, a single intravenous injection was performed. Group 1 is PBS, Group 2 is free anti-PD-1 and free R848, Group 3 is free anti-PD-1 and R848 loaded in untargeted nanoparticles (isotype control), and Group 4 is R848 loaded in PD-1-targeted nanoparticles. The dose was 20 μg for anti-PD-1 and 60 μg for R848. After 72 h, tumors were harvested, processed for immunohistochemistry, and analyzed by ImageJ software. Note that the effect is specific to CD8+ T cells, as the proportion of CD3+ remains unchanged (see FIG. 26).

FIG. 26 shows that the proportion of total CD3+ T cells remains unchanged following targeted delivery of a TLR7/8 agonist (R848) to PD-1-expressing cells. 200,000 MC38 cells were injected subcutaneously in 100 μl PBS into C57BL/6 mice on day 0. Fourteen days later, a single intravenous injection was performed. Group 1 is PBS, Group 2 is free anti-PD-1 and free R848, Group 3 is free anti-PD-1 and R848 loaded in untargeted nanoparticles (isotype control), and Group 4 is R848 loaded in PD-1-targeted nanoparticles. After 72 hours, tumors were harvested, processed for immunohistochemistry, and analyzed by ImageJ software.

FIGS. 27-29. Immunohistochemistry data showing that the tumors become inflamed with CD8 T+ cells if the TLR7/TLR8 agonist R848 is entrapped in PD-1-targeting nanoparticles.

FIG. 27 shows the percentage of area imaged with CD8+ and CD3+ T-cells under treatment with PBS, free anti-PD-1 and free R848, free anti-PD-1 or R848 loaded in untargeted nanoparticles (isotype control), and R848 loaded in PD-1-targeted nanoparticles.

FIG. 28 shows microscopy images of MC38 tumors with CD8+ T-cells with 40× magnification. Group 1 is treated with PBS, Group 2 is treated with free anti-PD-1 and free R848, Group 3 is treated with free anti-PD-1 and R848 loaded in untargeted nanoparticles (isotype control), and Group 4 is treated with R848 loaded in PD-1-targeted nanoparticles.

FIG. 29 shows microscopy images of MC38 tumors with CD3+ T-cells with 40× magnification. Group 1 is treated with PBS, Group 2 is treated with free anti-PD-1 and free R848, Group 3 is treated with free anti-PD-1 and R848 loaded in untargeted nanoparticles (isotype control), and Group 4 is treated with R848 loaded in PD-1-targeted nanoparticles.

FIG. 30. Optimization of F(ab′)2 conjugation to polymeric nanoparticles. FIG. 30A. Scheme of antibody fragment conjugation to the surface of pre-formulated maleimide-functionalized PEG-PLGA polymeric nanoparticles (NPs). FIG. 30B. A non-reducing SDS-PAGE gel stained with Coomassie Brilliant Blue is shown following IdeS-mediated cleavage of anti-CD8a and rat IgG2b isotype control antibodies. FIG. 30C A Western blot following reducing SDS-PAGE of CD8a-targeting NPs developed with Fab-specific (left panel) or Fc-specific antibodies (right panel); lane 1: uncoated NPs, lane 2: NPs without antibody reduction before conjugation, lane 3: anti-CD8 NPs with the antibody reduced using 0.5 mM DTT before conjugation, lane 4: anti-CD8 F(ab′)2 and Fc cleavage product as a positive control.

FIG. 31. CD8a-targeting nanoparticles bind to T cell in vitro and in vivo. FIG. 31A. CD8a-targeting NPs (loaded with DiD) bind to the surface of CD8+ T cells isolated from the spleen within 30 min of incubation, as assessed by flow cytometry. FIG. 31B. Quantification of DiD-positive T cells; data representative for more than 4 experiments. FIG. 31C. Timeline of in vivo binding experiment. FIG. 31D. Quantification of DiD-positive, CD3/CD8+ T cells 1, 24, and 48 h after the NPs were injected intravenously; n=3−6±SEM; ° Anti-CD8a antibody for flow cytometry staining could not bind due to steric hindrance with CD8a-targeting NPs. FIG. 31E. Quantification of CD3/CD8+ T cells in blood, spleen, tumor-draining lymph node (TdLN), and tumor 24 h after the NPs were injected intravenously.

FIG. 32. PD-1-targeting nanoparticles bind to T cells in vitro and in vivo. FIG. 32A. CD8+ OT-I T cells were activated with ovalbumin-expressing B16 (ratio 1:10 B16 to T cell) for 48 h and incubated with DiD-loaded, PD-1-targeting NPs for 30 min before detection of DiD by flow cytometry. FIG. 32B. C57BL/6 mice were inoculated with ovalbumin-expressing B16 melanoma cells. Once tumors reached ˜400mm³ in volume, DiD-loaded, PD-1-targeting NPs were injected intravenously. One hour later, tumors were recovered. Flow cytometry was performed (gating shown at left), and the percentage of T cells that positive for both PD-1 expression and NP binding was quantified (right panel).

FIG. 33. PD-1-targeting nanoparticles bind to activated human T cells. FIG. 33A. PD-1 expression on human CD3+ T cells following activation with anti-CD3/CD28 complex, n=4 independent donors±SEM. FIG. 33B. Dose-dependent binding of PD-1-targeting NPs to 250,000 activated human T cells. FIG. 33C. Quantification of T cells that were bound by DiD-loaded, PD-1-targeting NPs, μg per 250,000 T cells; graph shows results of two donors and is representative for at least two independent experiments. FIG. 33D. Pre-incubation of activated human T cells with free pembrolizumab (pre pembro) for 30 min blocks binding of PD-1-targeting NPs (10 μg/200,000 T cells), n=3±SD.

FIG. 34. Delivery of a TGFβR1 inhibitor (SD-208) from nanoparticles confers same phenotype as free drug in vitro. FIG. 34A. Proliferation of CD8+ T cells following activation with anti-CD3/CD28 beads (1:2 bead to T cell ratio) for 72 hours in the presence or absence of TGFβ1 (2 ng/mL); quantification of geometric mean of CTV in the right panel, n=3±SD. FIG. 34B. Intracellular granzyme B expression was assessed by flow cytometry, n=3±SD. FIG. 34C. Fold change of interferon-γ (IFNγ) was measured by ELISA, n=4±SEM.

FIG. 35. Targeted delivery of a TGFβR1 inhibitor (SD-208) to PD-1-expressing cells delays tumor growth and extends survival. 200,000 MC38 cells were injected subcutaneously into C57BL/6 mice on day 0. Five days later, NPs or free drugs were administered intravenously twice weekly up to a total of 7 injections. The dose was 20 μg of anti-PD-1 and 40 μg of SD-208. FIG. 35A. Tumor volume and FIG. 35B. animal survival were monitored to assess for efficacy.

FIG. 36. Targeted delivery of a TLR7/8 agonist (R848) to PD-1-expressing cells promotes infiltration of CD8+ T cells into MC38 tumors. 200,000 MC38 cells were injected subcutaneously into C57BL/6 mice on day 0. Fourteen days later, a single intravenous injection was performed with the following groups: 1) PBS, 2) anti-PD-1 IgG+free R848, 3) anti-PD-1 IgG+untargeted particles loaded with R848, 4) PD-1-targeting particles loaded with R848. The dose was 20 ug for anti-PD-1 and 60 ug for R848. After 72 hours, tumors were harvested, processed into FFPE blocks for immunohistochemistry or into single-cell suspensions for flow cytometry. FIG. 36A. Immunohistochemistry using anti-CD8 reveals that MC38 tumors are not highly inflamed at baseline. An increase in TILs (quantified in FIG. 36B using ImageJ software) is observed only if the TLR7/8 agonist is delivered via the targeted NPs. Flow cytometry analysis reveals that PD-1-targeted delivery of R848 increases the proportion of CD8+ T cells that produce FIG. 36C) granzyme B and FIG. 36D) IFNγ. The dose was 20 μg of anti-PD-1 and 60 μg of R848.

FIG. 37. T cells retain their ability to proliferate in co-culture with ovalbumin-expressing B16 melanoma cells in the presence of CD8-targeting nanoparticles. OT-I CD8+ T cells were incubated with anti-CD8a NPs (or relevant negative controls) for 30 min, washed to remove unbound NPs, and co-cultured with ovalbumin-expressing B16 tumor cells for 72 hours. Proliferation was assessed by CFSE dilution, and NP binding was assessed by fluorescence of DiD, which had been entrapped in the NP core.

FIG. 38. In vivo assessment of anti-CD8a nanoparticles. FIG. 38A. Gating strategy of in vivo binding experiment for blood, spleen, tumor, and TdLN. FIG. 38B. Percentage of NP-bound CD3+ T cells after NPs were in the circulation for 1 h, as described in FIG. 31.

FIG. 39. Characterization of PD-1-targeting nanoparticles. FIG. 39A. Non-reducing SDS-PAGE gel stained with Coomassie Brilliant Blue following enzymatic cleavage of anti-PD-1 and mouse IgG2a antibodies using IdeZ. FIG. 39B. Western blot after reducing SDS-PAGE of PD-1-targeting NPs developed with Fab-specific (left panel) or Fc-specific antibody (right panel); lane 1: uncoated NPs, lane 2: isotype NPs, lane 3: anti-PD-1 NPs, lane 4: anti-PD-1 F(ab′)2 and Fc cleavage product as positive control.

FIG. 40. Binding of PD-1-targeting nanoparticles to T cells activated by anti-CD3/CD28 beads. CD8+ OT-I T cells were activated with CD3/CD28 beads (ratio 1:2 beads to T cell) for 48 h and incubated with DiD-loaded, PD-1-targeting NPs for 30 min before detection of DiD by flow cytometry.

FIG. 41. Binding of PD-1-targeting NPs to T cells in B16 tumor-bearing mice. C57BL/6 mice were inoculated with ovalbumin-expressing B16 melanoma cells. Once tumors reached ˜400mm³ in volume, DiD-loaded, PD-1-targeting NPs were injected intravenously. One hour later, blood, spleen, and tumor-draining lymph nodes were recovered. Flow cytometry was performed (gating shown at left), and the percentage of T cells that were positive for both PD-1 expression and NP binding was quantified (right panel).

FIG. 42. Cleavage of Pembrolizumab and human IgG4 into F(ab′)2 and Fc using IdeS was confirmed. Non-reducing SDS-PAGE gel stained with Coomassie Brilliant Blue following enzymatic cleavage of Pembrolizumab and human IgG4 antibodies using IdeS.

FIG. 43. Analysis of SD-208-encapsulating nanoparticles. FIG. 43A. Absorbance scan of SD-208 for the determination of drug encapsulation. FIG. 43B. Release profile of SD-208 containing NPs in 10% FBS in PBS, n=3±SD.

FIG. 44. Binding of GITR-targeting nanoparticles to T cells in B16 tumor-bearing mice. C57BL/6 mice were inoculated with B16 melanoma cells. Once tumors reached ˜400mm³ in volume, DiD-loaded, GITR-targeting NPs were injected intravenously. Two hours later, tumors were recovered. Flow cytometry was performed. FIG. 44A. Gating of CD4+ T cells on GITR+ and DiD+ is shown. FIG. 44B. The percentage of CD4+ T cells that were positive for both GITR expression and NP binding was quantified. FIG. 44C. Gating of CD8+ T cells on GITR+ and DiD+ is shown. FIG. 44D. The percentage of CD8+ T cells that were positive for both GITR expression and NP binding was quantified. FIG. 44E. Note that there were ˜10-fold fewer CD8+ T cells than CD4+ T cells recovered from the tumors.

FIG. 45. Binding of Gr-1-targeting nanoparticles to Ly-6C+ myeloid-derived suppressor cells in B16 tumor-bearing mice. C57BL/6 mice were inoculated with B16 melanoma cells. Once tumors reached ˜400mm³ in volume, DiD-loaded, Gr-1-targeting NPs were injected intravenously. Two hours later, tumors were recovered. Flow cytometry was performed. FIG. 45A. Gating of CD11b+ myeloid cells on Ly-6C+ and DiD+ is shown. The HK1.4 clone (used for flow cytometry does not block the binding of clone) and RB6-8C5 clone (used for targeting to Gr-1).) do not compete for binding to Ly-6C. FIG. 45B. The percentage of CD11b+ myeloid cells that were positive for both Gr-1 expression and NP binding was quantified. FIG. 45C. Note that there were ˜10-fold fewer Ly-6G+ myeloid cells than Ly-6C+ myeloid cells recovered from the tumors.

FIG. 46. The F(ab′)2-conjugated targeting nanoparticles described herein are not phagocytosed by macrophages. C57BL/6 mice were inoculated with B16 melanoma cells. Once tumors reached ˜400mm³ in volume, DiD-loaded, Gr-1-targeting NPs were injected intravenously. Two hours later, tumors were recovered. Flow cytometry was performed. CD11b+ myeloid cells gated on F4/80+ and DiD+ are shown. In the absence of Fc (IgG constant regions), the particles are not recognized by Fc receptors expressed on macrophages.

### DETAILED DESCRIPTION OF CERTAIN EMBODIMENTS OF THE INVENTION

One aspect of the present disclosure relates to a particle comprising a core containing at least one pharmaceutically active agent and an antibody or fragment thereof conjugated to the surface of the particle, wherein the antibody or fragment thereof targets a T-cell. In some embodiments, the antibody or fragment thereof is an antibody fragment. In some embodiments, the antibody fragment is enzymatically produced by fragmentation of an intact antibody using IdeS or IdeZ. In some embodiments, the antibody fragment is enzymatically produced by fragmentation of an intact antibody using IdeS or IdeZ has a defined sequence. In some embodiments, the antibody or fragment thereof is directly conjugated to the surface of the particle. In some embodiments, the particle is not an artificial antigen presenting cell. In some embodiments, the particles are not artificial antigen presenting cells.

In some embodiments, the antibody or fragment thereof targets a specific immune cell and delivers the pharmaceutically active agent to the specific immune cell (e.g., T-cell). In some embodiments, the antibody fragment targets a specific immune cell and delivers the pharmaceutically active agent to cells in the surrounding microenvironment. In some embodiments, the method includes targeting a T-cell to deliver pharmaceutical agents to cells in the tumor microenvironment or draining lymph node for the treatment of proliferative disease.

In some embodiments, the particle comprises a corona around at least a portion of the surface of the particle core. In some embodiments, the corona comprises a polymer. In some embodiments, the corona comprises polyethylene glycol (PEG). In some embodiments, the corona has a moiety allowing for attachment of the antibody fragment to the surface of the particle. In some embodiments, the PEG corona has a moiety allowing for attachment of the antibody fragment to the surface of the particle. In certain embodiments, the moiety is an electrophile-PEG corona. In certain embodiments, the electrophile-PEG corona is a maleimide-PEG corona. In certain embodiments, the maleimide-PEG corona allows for attachment of the antibody fragment to the surface of the particle. In some embodiments, the particle comprises a coating covering at least a portion of the surface of the particle core. In some embodiments, the coating comprises a polymer. In some embodiments, the coating comprises polyethylene glycol (PEG). In some embodiments, the PEG coating has a moiety allowing for attachment of the antibody or fragment thereof. In certain embodiments, the moiety is an electrophile-PEG corona. In certain embodiments, the electrophile-PEG corona is a maleimide-PEG corona. In certain embodiments, the PEG coating has a maleimide-PEG corona, which allows for attachment of the antibody or fragment thereof to the surface of the particle. In some embodiments, the antibody or fragment thereof is directly conjugated to the surface of the particle. In some embodiments, the antibody or fragment thereof is directly conjugated to the PEG-PLGA nanoparticle. In some embodiments, the antibody or fragment thereof is covalently bound to the surface of the particle. In some embodiments, the antibody or fragment thereof is not non-covalently bound to the surface of the particle. In some embodiments, the antibody or fragment thereof is not non-covalently bound (e.g., biotin/streptavidin binding) to the surface of the particle. In some embodiments, the antibody or fragment thereof is not non-covalently bound (e.g., biotin/streptavidin binding) to the PEG-PLGA nanoparticle. In some embodiments, the antibody or fragment thereof is covalently bound to the PEG-PLGA nanoparticle. In certain embodiments, the antibody or fragment thereof attached to the particle targets specific T-cells. In certain embodiments, the antibody or fragment thereof attached to the particle targets specific T-cells in vivo. In certain embodiments, the antibody or fragment thereof attached to the particle targets specific T-cells, enabling the delivery of the pharmaceutically active agent contained in the particle to specific T-cells. In certain embodiments, the antibody or fragment thereof attached to the particle targets particular T-cells, allowing the delivery of the pharmaceutically active agent within the particle to particular T-cells or to tissues in which such T cells reside or to tissues to which such T-cells migrate. In certain embodiments, the particle is internalized by the T-cell. In certain embodiments, the particle is internalized by activated T-cells. In certain embodiments, the particle is internalized by activated CD8+ T-cells.

In some embodiments, the antibody or fragment thereof is a F(ab′)₂ fragment. In some embodiments, the antibody or fragment thereof is a Fab fragment. In some embodiments, the antibody or fragment thereof is a Fab′ fragment.

In some embodiments, the antibody or fragment thereof is an antibody fragment. In some embodiments, the antibody fragment is enzymatically produced by fragmentation of an intact antibody using IdeS or IdeZ. In some embodiments, the antibody fragment is enzymatically produced by fragmentation of an intact antibody using IdeS or IdeZ has a defined sequence. In some embodiments, the antibody fragment is a F(ab′)2.

In some embodiments, the antibody or fragment thereof targets an endogenous immune cell subset. In some embodiments, the endogenous immune cell subset is a myeloid-derived suppressor cell. In some embodiments, the antibody or fragment thereof targets a marker expressed on the surface of myeloid-derived suppressor cells (MDSC). In some embodiments, the marker expressed on the surface of MDSC's is Gr-1.

In some embodiments, the antibody or fragment thereof targets endogenous T-cells. In some embodiments, the antibody or fragment thereof targets a surface antigen on the endogenous T-cells. In some embodiments, the target of the antibody or fragment thereof is selected from the group consisting of PD-1, Thyl.1, CD8, CD137, LAG-3, and TIM-3. In some embodiments, the target of the antibody or fragment thereof is selected from the group consisting of PD-1, CD8, CD25, CD27, LAG-3, TIM-3, BTLA, VISTA, TIGIT, NRP1, TNFRSF25, OX40, GITR, and ICOS. In some embodiments, the target of the antibody or fragment thereof is found on other cells (e.g., Natural Killer (NK) cells). In some embodiments, PD-1, Thy1.1, CD8, CD137, LAG-3, or TIM-3 will also be targeted on NK cells because the NK cells express these markers. In some embodiments, PD-1, CD8, CD25, CD27, LAG-3, TIM-3, BTLA, NRP1, TNFRSF25, OX40, GITR, or ICOS will also be targeted on NK cells because the NK cells express these markers. In some embodiments, the target of the antibody or fragment thereof is PD-1. In some embodiments, the target of the antibody or fragment thereof is Thy1.1. In some embodiments, the target of the antibody or fragment thereof is CD8. In some embodiments, the target of the antibody or fragment thereof is CD137. In some embodiments, the target of the antibody or fragment thereof is LAG-3. In some embodiments, the target of the antibody or fragment thereof is TIM-3. In some embodiments, the target of the antibody or fragment thereof is CD25. In some embodiments, the target of the antibody or fragment thereof is CD27. In some embodiments, the target of the antibody or fragment thereof is BTLA. In some embodiments, the target of the antibody or fragment thereof is VISTA. In some embodiments, the target of the antibody or fragment thereof is TIGIT. In some embodiments, the target of the antibody or fragment thereof is NRP1. In some embodiments, the target of the antibody or fragment thereof is TNFRSF25. In some embodiments, the target of the antibody or fragment thereof is OX40. In some embodiments, the target of the antibody or fragment thereof is GITR. In some embodiments, the target of the antibody or fragment thereof is ICOS.

In some embodiments, the T-cell is an endogenous T-cell. In some embodiments, the T-cell is a CD8+ T-cell. In some embodiments, the T-cell is a tumor-reactive T-cell. In some embodiments, the T-cell is a tumor-specific T-cell. In some embodiments, the T-cell is a CD4+ T-cell. In some embodiments, the T-cell is a regulatory T-cell.

In some embodiments, the antibody or fragment thereof targets CD8+ T-cells. In some embodiments, the antibody or fragment thereof targets PD-1+ T-cells. In some embodiments, PD-1+ T-cells represent a subset of T-cells that have become activated and then exhausted. In some embodiments, the subset of T-cells that have become activated are not later exhausted. In some embodiments, the antibody or fragment thereof targets a subset of NK cells that have become activated and then exhausted. In some embodiments, the subset of NK cells that have become activated are not later exhausted. In some embodiments, the antibody or fragment thereof targets CD4+ T-cells. In certain embodiments, the antibody or fragment thereof targets regulatory CD4+ T-cells. In some embodiments, an antibody or fragment thereof targets GITR. In some embodiments, the antibody or fragment thereof targets GITR+ T-cells. In certain embodiments, the particle comprises two antibodies or fragments thereof. In some embodiments, an antibody or fragment thereof targets CD8. In some embodiments, a second antibody or fragment thereof targets PD-1. In some embodiments, one antibody or fragment thereof targets PD-1. In some embodiments, a second antibody or fragment thereof targets CD137. In some embodiments, a second antibody fragment targets GITR.

In some embodiments, the target of the antibody or fragment thereof is a marker expressed on the surface of myeloid-derived suppressor cells (MDSC). In some embodiments, the target of the antibody or fragment thereof is Gr-1. Gr-1, or its human equivalent, may include but is not limited to CCR2, CD11b, CD14, CD15, CD33, CD39, CD66b, CD124, IL4Ra, and/or S100 family members, including S100A8, S100A9, S10A12. In some embodiments, the target of the antibody or fragment thereof is CCR2, CD11b, CD14, CD15, CD33, CD39, CD66b, CD124, IL4Ra, and/or S100 family members, including S100A8, S100A9, S 10Al2. In certain embodiments, an antibody or fragment thereof targeting two of these receptors is used.

In some embodiments, the antibody or fragment thereof targets a peripheral T-cell. In some embodiments, the antibody or fragment thereof targets a tumor-resident T-cell. In some embodiments, the antibody or fragment thereof targets an activated T-cell. In some embodiments, the antibody or fragment thereof targets an activated CD8+ T-cell. In some embodiments, the antibody or fragment thereof targets an activated CD4+ T-cell. In some embodiments, the antibody or fragment thereof targets a tumor-specific T-cell. In some embodiments, the antibody or fragment thereof targets a tumor-specific T-cell in vivo.

In some embodiments, the antibody or fragment thereof targets an effector T-cell. In some embodiments, the antibody or fragment thereof targets a regulatory T-cell. In some embodiments, the antibody or fragment thereof targets a regulatory T-cell in vivo. In some embodiments, the antibody fragment targets a regulatory T-cell in vivo. In some embodiments, the antibody or fragment thereof targets a suppressor cell. In some embodiments, the antibody or fragment thereof targets a myeloid-derived suppressor cell. In some embodiments, the antibody fragment targets a myeloid-derived suppressor cell. In some embodiments, the antibody or fragment thereof targets a myeloid-derived suppressor cell (MDSC) in vivo. In some embodiments, the antibody fragment targets a myeloid-derived suppressor cell (MDSC) in vivo. In some embodiments, the antibody or fragment thereof targets a monocytic MDSC. In some embodiments, the antibody fragment targets a monocytic MDSC. In some embodiments, the antibody or fragment thereof targets a granulocytic MDSC. In some embodiments, the antibody fragment targets a granulocytic MDSC.

In some embodiments, the particles, described herein, may have a relatively small diameter. In certain embodiments, the particle is a nanoparticle. In certain embodiments, the average cross-sectional dimension of the particle ranges from 200 to 500 nm. In some embodiments, the average cross-sectional dimension of the particle ranges from 250 to 300 nm. As used herein, the diameter of a particle for a non-spherical particle is the diameter of a perfect mathematical sphere having the same volume as the non-spherical particle. In general, the particles are approximately spherical; however the particles are not necessarily spherical but may assume other shapes (e.g., discs, rods) as well. The measurements described herein typically represent the average particle size of a population. However, in certain embodiments, the measurements may represent the range of sizes found in a population, or the maximum or minimum size of particles found in the population. In some embodiments, the diameter of the core may fall within the above-mentioned ranges for the size of the particle.

In some embodiments, the core contains more than one pharmaceutically active agent. In some embodiments, the core contains a second pharmaceutically active agent. In some embodiments, the core contains a single pharmaceutically active agent (e.g., biological macromolecule, or small molecule). In some embodiments, the core contains a single pharmaceutically active agent (e.g., small molecule). In some embodiments, the core contains two or more pharmaceutically active agents. In certain embodiments, the core contains two or more pharmaceutically active agents, such as a small molecule and a biological macromolecule, two or more small molecules, or two or more biological molecules. In certain embodiments, the core contains two or more pharmaceutically active agents, such as a two or more small molecules. In certain embodiments, the core contains two or more biological molecules. In some embodiments, the core may contain two or more small molecules.

In some embodiments, the pharmaceutically active agent is a small molecule. In some embodiments, the small molecule is hydrophobic. In some embodiments, the pharmaceutically active agent is an immunomodulatory compound. In some embodiments, the immunomodulatory compound is a kinase inhibitor. In some embodiments, the kinase inhibitor is selected from the group consisting of: transforming growth factor β receptor I (TGF-βR I) kinase inhibitor, mammalian target of rapamycin (mTOR) inhibitor, glycogen synthase kinase-3β (GSK-3β) inhibitor, diacylglycerol kinase (DGK) inhibitor, and combinations thereof. In some embodiments, the kinase inhibitor is selected from the group consisting of: transforming growth factor β receptor I (TGF-βR I) kinase inhibitor, mammalian target of rapamycin (mTOR) inhibitor, glycogen synthase kinase-3β (GSK-3β) inhibitor, diacylglycerol kinase (DGK) inhibitor, proto-oncogene serine/threonine-protein kinase (PIM) inhibitor, phosphatidyl-inositol-3 kinase (PI3K) inhibitor, Janus kinase (JAK) inhibitor, mitogen-activated protein kinase (MEK) inhibitor, and combinations thereof. In some embodiments, the immunomodulatory compound is a TGF-βR I kinase inhibitor. In some embodiments, the immunomodulatory compound is an mTOR inhibitor. In some embodiments, the immunomodulatory compound is a GSK-3β inhibitor. In some embodiments, the immunomodulatory compound is a DGK inhibitor. In some embodiments, the immunomodulatory compound is a PIM inhibitor. In some embodiments, the PIM inhibitor is PIM447. In some embodiments, the immunomodulatory compound is a PI3K inhibitor. In some embodiments, the PI3K inhibitor is BKM120. In some embodiments, the immunomodulatory compound is specific for PI3Kγ. In some embodiments, the immunomodulatory compound is specific for PI3Kδ. In some embodiments, the immunomodulatory compound is a Janus kinase (JAK) inhibitor. In some embodiments, the JAK inhibitor is ruxolitinib, and has the structure:

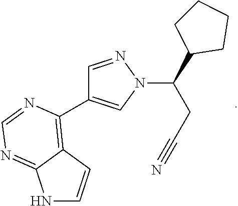 [SMILES: N#CC[C@H](C1CCCC1)n1cc(-c2ncnc3[nH]ccc23)cn1]

In some embodiments, the immunomodulatory compound is a MEK inhibitor.

In some embodiments, the immunomodulatory compound is a IDO1 inhibitor. In some embodiments, the immunomodulatory compound is a TDO2 inhibitor. In some embodiments, the IDO inhibitor is Epacadostat, with the structure:

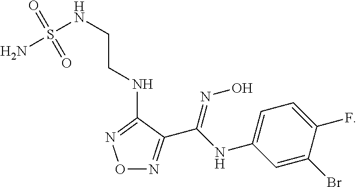 [SMILES: NS(=O)(=O)NCCNc1nonc1/C(=N/O)Nc1ccc(F)c(Br)c1]

In some embodiments, the immunomodulatory compound is a ARG1 inhibitor. In some embodiments, the immunomodulatory compound is a PGE2 inhibitor. In some embodiments, the immunomodulatory compound is a PDE5 inhibitor. In some embodiments, the immunomodulatory compound is a COX2 inhibitor. In some embodiments, the immunomodulatory compound is an IAP inhibitor. In some embodiments, the IAP inhibitor is LCL161. In some embodiments, the immunomodulatory compound is a SHP-1 inhibitor. In some embodiments, the immunomodulatory compound is a SHP-2 inhibitor. In some embodiments, the immunomodulatory compound is a PORCN inhibitor. In some embodiments, the PORCN inhibitor is WNT974. In some embodiments, the immunomodulatory compound is a A2AR inhibitor. In some embodiments, the PI3K inhibitor is NIR178. In some embodiments, the immunomodulatory compound is a CSF1R inhibitor. In some embodiments, the immunomodulatory compound is a RON inhibitor. In some embodiments, the TGF-βR I kinase inhibitor is a compound comprising the structure:

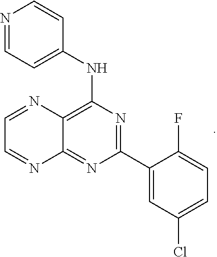 [SMILES: Fc1ccc(Cl)cc1-c1nc(Nc2ccncc2)c2nccnc2n1]

In certain embodiments, the pharmaceutically active agent is an inhibitor of TGFβ signaling. In certain embodiments, the pharmaceutically active agent is an inhibitor of the TGFβ receptor I kinase. In certain embodiments, the pharmaceutically active agent binds to the TGFβ receptor I kinase. In certain embodiments, the pharmaceutically active agent specifically binds to the TGFβ receptor I kinase. In certain embodiments, the pharmaceutically active agent is compound SD-208. In certain embodiments, the pharmaceutically active agent is proto-oncogene serine/threonine-protein kinase (PIM) inhibitor. In certain embodiments, the pharmaceutically active agent is phosphatidyl-inositol-3 kinase (PI3K) inhibitor. In certain embodiments, the pharmaceutically active agent is Janus kinase (JAK) inhibitor. In certain embodiments, the pharmaceutically active agent is mitogen-activated protein kinase (MEK) inhibitor.

In some embodiments, the immunomodulatory compound is not a kinase inhibitor. In some embodiments, the non-kinase inhibitor is selected from the group consisting of: indoleamine 2,3-dioxygenase (IDO1) inhibitor, tryptophan 2,3-dioxygenase (TDO2) inhibitor, arginase (ARG1) inhibitor, prostaglandin E2 (PGE2) inhibitor, phosphodiesterase type 5 (PDE5) inhibitor, cyclooxygenase-2 (COX2) inhibitor, inhibitors of apoptosis proteins (IAP) inhibitor, Src homology region 2 domain-containing phosphatase-1 (SHP-1) inhibitor, Src homology region 2 domain-containing phosphatase-2 (SHP-2) inhibitor, porcupine homology (PORCN) inhibitor, adenosine A2A receptor (A2AR) inhibitor, colony-stimulating factor 1 receptor (CSF1R) inhibitor, macrophage-stimulating protein receptor (RON) inhibitor, and combinations thereof. In certain embodiments, the immunomodulatory compound is IDO1 inhibitor. In certain embodiments, the immunomodulatory compound is TDO2 inhibitor. In certain embodiments, the immunomodulatory compound is ARG1 inhibitor. In certain embodiments, the immunomodulatory compound is PGE2 inhibitor. In certain embodiments, the immunomodulatory compound is phosphodiesterase type 5 (PDE5) inhibitor.

In some embodiments, the immunomodulatory compound is an activator of innate immunity. In certain embodiments, the pharmaceutically active agent is an agonist of a toll-like receptor (TLR). In some embodiments, the immunomodulatory compound is a TLR2 agonist, TLR4 agonist, TLR7 agonist, a TLR8 agonist, and combinations thereof. In certain embodiments, the pharmaceutically active agent is a TLR7 agonist. In certain embodiments, the pharmaceutically active agent is a TLR8 agonist. In certain embodiments, the pharmaceutically active agent is an agonist of TLR7 and TLR8. In certain embodiments, the pharmaceutically active agent is resiquimod (R848). In certain embodiments, the pharmaceutically active agent is an immunomodulatory compound that is an agonist of a Toll-like receptor (TLR), a C-type lectin receptor (CLR), or a NOD-like receptor (NLR) selected from the group consisting of: TLR2 agonist, TLR4 agonist, TLRS agonist, TLR7 agonist, TLR8 agonist, Dectin-1 agonist, Dectin-2 agonist, Mincle agonist, NOD1 agonist, NOD2 agonist, and combinations thereof. In certain embodiments, the pharmaceutically active agent increases the proportion of CD8⁺ T cells in the tumor. In certain embodiments, targeted delivery of a TLR agonist to PD-1+ T cells inflames a non-T-cell-inflamed tumor, which improves patient response to cancer immunotherapy.

In some embodiments, the pharmaceutically active agent is a biological macromolecule. In some embodiments, the biological macromolecule is a nucleic acid. In some embodiments, the biological macromolecule is a peptide. In some embodiments, the biological macromolecule is an antibody or fragment thereof. In certain embodiments, the pharmaceutically active agent is not a biologic. In certain embodiments, the pharmaceutically active agent is not an anti-CD137 antibody. In certain embodiments, the pharmaceutically active agent is not interleukin-2 (IL-2). In certain embodiments, the pharmaceutically active agent is not an IL-2-Fc fusion protein. In certain embodiments, the pharmaceutically active agent is not a vaccine. In certain embodiments, the pharmaceutically active agent is not a source of antigen for vaccination.

In some embodiments, the weight percentage of a single pharmaceutically active agent (e.g., pharmaceutically active agent) and/or of all the pharmaceutically active agents in the particles (i.e., loading efficiency) is at least about 0.5%, at least about 1%, at least about 2%, at least about 4%, at least about 6%, at least about 8%, at least about 10%, at least about 15%, at least about 20%, at least about 25%, at least about 30%, at least about 35%, at least about 40%, at least about 45%, or at least about 50%, all as percentage by weight. In some embodiments, the loading efficiency is between about 0.5% and about 60%, between about 0.5% and about 50%, between about 0.5% and about 40%, between about 0.5% and about 30%, between about 1% and about 60%, between about 1% and about 50%, between about 1% and about 40%, between about 1% and about 30%, between about 2% and about 60%, between about 2% and about 50%, between about 2% and about 40%, or between about 2% and about 30%, all as percentage by weight. The loading efficiency may be determined by extracting the pharmaceutically active agent from the dried particles using, e.g., organic solvents, and measuring the quantity of the agent using high pressure liquid chromatography (i.e., HPLC), liquid chromatography-mass spectrometry, nuclear magnetic resonance, absorbance, fluorescence, or mass spectrometry. Those of ordinary skill in the art would be knowledgeable of techniques to determine the quantity of an agent using the above-referenced techniques. For example, HPLC may be used to quantify the amount of an agent by, e.g., comparing the area under the curve of a HPLC chromatogram to a standard curve.

In some embodiments, the pharmaceutically active agent is encapsulated by the polymer in the core. In certain embodiments, the core of the particle is substantially solid.

In certain embodiments, the core of the particle comprises a biodegradable polymer. In some embodiments, the core comprises one or more hydrolytically degradable polymers. As used herein, “biodegradable” particles are particles that, when introduced into cells, are broken down by the cellular machinery or by hydrolysis into components that the cells can either reuse or dispose of without significant toxic effects on the cells, i.e., fewer than about 20% (e.g., fewer than about 15%, fewer than about 10%, fewer than about 5%, fewer than about 3%, fewer than about 2%, fewer than about 1%) of the cells are killed when the components are added to cells in vitro. The components preferably do not cause inflammation or other adverse effects in vivo. In certain embodiments, the chemical reactions relied upon to break down the biodegradable particles are catalyzed. In other embodiments, the chemical reactions relied upon to break down the biodegradable particles are not catalyzed. The particle may degrade over hours to days to weeks to months, thereby releasing the agent (e.g., pharmaceutically active agent) over an extended period of time. In certain embodiments, the half-life of the particle under physiological conditions is 1-72 hours (e.g., 1-48 hours, 1-24 hours). In certain embodiments, the half-life of the particle under physiological conditions is 1-7 days. In other embodiments, the half-life is from 2-4 weeks. In other embodiments, the half-life is approximately 1 month.

In some embodiments, the core comprises a synthetic polymer (e.g., polyester). An exemplary, non-limiting list of polymers that may be used to form the core includes polyesters such as poly(lactic acid)/polylactide, poly(glycolic acid), poly(lactic-co-glycolic acid), and poly(caprolactone); poly(orthoesters); poly(anhydrides); poly(ether esters) such as polydioxanone; poly(carbonates); poly(amino carbonates); and poly(hydroxyalkanoates) such as poly(3-hydroxybutyrate) and poly(3-hydroxybutyrate-co-3-hydroxyvalerate); polyphosphazenes; polyacrylates; poly(alkyl acrylates); polyamides; polyamines such as poly(amido amine) dendrimers; polyethers; poly(ether ketones); poly(alkaline oxides) such as polyethylene glycol; polyacetylenes and polydiacetylenes; polysiloxanes; polyolefins; polystyrene such as sulfonated polystyrene; polycarbamates; polyureas; polyimides; polysulfones; polyurethanes; polyisocyanates; polyacrylonitriles; polysaccharides such as alginate and chitosan; polypeptides; and derivatives and block, random, radial, linear, and teleblock copolymers, and blends of the above. In some embodiments, poly(lactic-co-glycolic acid) is used to form the core.

The polymers may be homopolymers or copolymers. Other potentially suitable polymer molecules are described in the Polymer Handbook, Fourth Ed., Brandrup, J. Immergut, E. H., Grulke, E. A., Eds., Wiley-Interscience: 2003, which is incorporated herein by reference in its entirety.

The polymers are generally extended molecular structures comprising backbones which optionally contain pendant side groups or chains, wherein the term backbone is given its ordinary meaning as used in the art, e.g., a linear chain of atoms within the polymer by which other chains may be regarded as being pendant. Typically, but not always, the backbone is the longest chain of atoms within the polymer. A polymer may be a co-polymer, for example, a block, alternating, or random co-polymer. Polymers may be obtained from natural sources or be created synthetically. In some embodiments, the polymer may be acyclic or cyclic. In some embodiments, the polymers in the core are not cross-linked. In other embodiments, the polymers in the core are cross-linked.

In certain embodiments, the polymer is poly(lactic-co-glycolic acid) (PLGA). In certain embodiments, the polymer is poly(lactic acid). In certain embodiments, the polymer is poly(glycolic acid). In certain embodiments, the polymer is poly(lactic-co-glycolic acid)-poly(ethylene glycol) copolymer. In certain embodiments, the polymer is poly(lactic acid)-poly(ethylene glycol) copolymer. In certain embodiments, the polymer is poly(glycolic acid)-poly(ethylene glycol) copolymer. In certain embodiments, the polymer comprises combinations of synthetic polymers. In certain embodiments, the polymer comprises combinations of poly(lactic-co-glycolic acid), poly(lactic acid), poly(glycolic acid), poly(lactic-co-glycolic acid)-poly(ethylene glycol) copolymer, poly(lactic acid)-poly(ethylene glycol) copolymer, and poly(glycolic acid)-poly(ethylene glycol) copolymer. In certain embodiments, the polymer is PLGA (with a molecular weight (MW) ranging from 10 to 15 kDa) and a 50:50 ratio of poly(lactic acid) to poly(glycolic acid). In certain embodiments, 75% of this polymer mixture is blended with 25% maleimide-functionalized PEG-PLGA (10 kDa MW) and a 50:50 ratio of poly(lactic acid) to poly(glycolic acid), where PEG has a 5 kDa chain length. In certain embodiments, the polymer is PLGA with a MW of 30 kDa. In certain embodiments, the polymer is PLGA with a MW of 40 kDa. In certain embodiments, the polymer is PLGA with 100% poly(lactic acid). In certain embodiments, the polymer is PLGA with a 75:25 ratio of poly(lactic acid) to poly(glycolic acid). In some embodiments, the core comprises a mixture of two or more polymers.

In some embodiments, the particle has an encapsulating efficiency of over 50% of the pharmaceutically active agent. In some embodiments, the particle has an encapsulating efficiency of over 60% of the pharmaceutically active agent. In some embodiments, the particle has an encapsulating efficiency of 60-70% of the pharmaceutically active agent. In some embodiments, the particle has an encapsulating efficiency of 60-65% of the pharmaceutically active agent. In some embodiments, the particle has an encapsulating efficiency of 65-70% of the pharmaceutically active agent. In some embodiments, the particle has an encapsulating efficiency of 50-60% of the pharmaceutically active agent. In some embodiments, the particle has an encapsulating efficiency of 50-70% of the pharmaceutically active agent. In some embodiments, the particle has an encapsulating efficiency of 50-55% of the pharmaceutically active agent. In some embodiments, the particle has an encapsulating efficiency of 55-60% of the pharmaceutically active agent.

In some embodiments, the particle has an encapsulating efficiency of below 50% of the pharmaceutically active agent. In some embodiments, the particle has an encapsulating efficiency of less than 30% of the pharmaceutically active agent. In some embodiments, the particle has an encapsulating efficiency of less than 20% of the pharmaceutically active agent. In some embodiments, the particle has an encapsulating efficiency of 5-30% of the pharmaceutically active agent. In some embodiments, the particle has an encapsulating efficiency of 5-10% of the pharmaceutically active agent. In some embodiments, the particle has an encapsulating efficiency of 10-20% of the pharmaceutically active agent. In some embodiments, the particle has an encapsulating efficiency of 20-30% of the pharmaceutically active agent. In some embodiments, the particle has an encapsulating efficiency of 10-15% of the pharmaceutically active agent. In some embodiments, the particle has an encapsulating efficiency of 15-20% of the pharmaceutically active agent. In some embodiments, the particle has an encapsulating efficiency of below 25% of the pharmaceutically active agent. In some embodiments, the particle has an encapsulating efficiency of between 1-25% of the pharmaceutically active agent. In some embodiments, the particle has an encapsulating efficiency of between 1-20% of the pharmaceutically active agent. In some embodiments, the particle has an encapsulating efficiency of between 5-25% of the pharmaceutically active agent. In some embodiments, the particle has an encapsulating efficiency of between 5-20% of the pharmaceutically active agent. In some embodiments, the particle has an encapsulating efficiency of between 5-15% of the pharmaceutically active agent. In some embodiments, the particle has an encapsulating efficiency of between 5-10% of the pharmaceutically active agent.

The particle may optionally include other components (e.g., chemical compounds, coatings), in addition to the core and the antibody or fragment thereof conjugated to the surface of the particle. In some embodiments, the particle comprises a surface modifying agent on the surface of the particle. Examples of surface modifying agents include polymers (e.g., polyethylene glycol). In certain embodiments, the surface modifying agent is polyethylene glycol. In certain embodiments, the surface modifying agent is a co-polymer of polyethylene glycol. In certain embodiments, the surface modifying agent changes the surface characteristics of the particle.

Another aspect relates to methods of preparing the particles described herein. In certain embodiments, the method comprises providing a polymeric core containing a pharmaceutically active agent; and conjugating an antibody or fragment thereof to the surface of the particle, wherein the antibody or fragment thereof targets a T-cell. In certain embodiments, the method comprises providing a polymeric core containing a pharmaceutically active agent; and conjugating an antibody fragment to the surface of the particle, wherein the antibody fragment thereof targets a T-cell. In certain embodiments, before an antibody or fragment thereof is conjugated to the surface of the particle, the antibody or fragment thereof is first treated with an immunoglobulin-degrading enzyme, and reduced with a reducing agent, (e.g., dithiothreitol (DTT)). In certain embodiments, before an antibody fragment is conjugated to the surface of the particle, the antibody fragment is first treated with an immunoglobulin-degrading enzyme, and reduced with a reducing agent. In certain embodiments, the immunoglobulin-degrading enzyme is IdeS enzyme (e.g., FabRICATOR). In certain embodiments, the immunoglobulin-degrading enzyme is IdeZ enzyme. In certain embodiments, the step of conjugating the antibody or fragment thereof to the surface of the particle comprises attaching an electrophile to a PEG corona on the surface of the particle; and conjugating the antibody or fragment thereof to the electrophile-PEG corona on the surface of the particle. In certain embodiments, the step of conjugating the antibody or fragment thereof to the surface of the particle comprises attaching an electrophile to a PEG corona on the surface of the particle; and conjugating the antibody fragment to the electrophile-PEG corona on the surface of the particle. In certain embodiments, the electrophile is maleimide. In certain embodiments, maleimide is attached to a PEG corona on the surface of the particle, and the antibody or fragment thereof is conjugated to the maleimide-PEG corona on the surface of the particle. In certain embodiments, maleimide is attached to a PEG corona on the surface of the particle, and the antibody fragment is conjugated to the maleimide-PEG corona on the surface of the particle. In some embodiments, the antibody or fragment thereof is directly conjugated to the surface of the particle. In some embodiments, the antibody or fragment thereof is directly conjugated to the PEG-PLGA nanoparticle. In some embodiments, the antibody or fragment thereof is not non-covalently bound to the surface of the particle. In some embodiments, the antibody or fragment thereof is covalently bound to the surface of the particle. In some embodiments, the antibody or fragment thereof is derived from nivolumab, pembrolizumab, PDR001, MBG453, LAG525, or GWN323. In some embodiments, the antibody fragment is derived from nivolumab, pembrolizumab, PDR001, MBG453, LAG525, or GWN323. In some embodiments, the antibody or fragment thereof targets GITR or Gr-1. In some embodiments, the antibody fragment targets GITR or Gr-1. In some embodiments, the target of the antibody or fragment thereof is CCR2, CD11b, CD14, CD15, CD33, CD39, CD66b, CD124, IL4Ra, and/or S100 family members, including S100A8, S100A9, S10A12. In certain embodiments, an antibody or fragment thereof targets two of these receptors.

Once the particles have been prepared, the prepared particles may be combined with pharmaceutically acceptable excipients to form a pharmaceutical composition. Another aspect of the invention relates to a pharmaceutical composition, wherein the pharmaceutical composition comprises a plurality of particles and a pharmaceutically acceptable excipient. In some embodiments, the pharmaceutical composition comprises a therapeutically effective amount of the particle for use in treating a proliferative disease in a subject in need thereof. In some embodiments, the proliferative disease is cancer. Once the particles have been prepared, they may be combined with pharmaceutically acceptable excipients to form a pharmaceutical composition. As would be appreciated by one of skill in this art, the excipients may be chosen based on the route of administration as described below, the agent being delivered, and the time course of delivery of the agent.

Pharmaceutical compositions of the present disclosure and for use in accordance with the present invention may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The pharmaceutical compositions of this invention can be administered to humans and/or to animals, orally, rectally, parenterally, intracisternally, intravaginally, intranasally, intraperitoneally, topically (as by powders, creams, ointments, or drops), bucally, or as an oral or nasal spray.

Liquid dosage forms for oral administration include pharmaceutically acceptable emulsions, microemulsions, solutions, suspensions, syrups, and elixirs. In addition to the active ingredients (i.e., the particles), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3 butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

Compositions for rectal or vaginal administration are preferably suppositories which can be prepared by mixing the inventive particles with suitable non irritating excipients or carriers such as cocoa butter, polyethylene glycol, or a suppository wax which are solid at ambient temperature but liquid at body temperature and therefore melt in the rectum or vaginal cavity and release the microparticles.

Solid dosage forms for oral administration include capsules, tablets, pills, powders, and granules. In such solid dosage forms, the particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of an inventive pharmaceutical composition include ointments, pastes, creams, lotions, gels, powders, solutions, sprays, inhalants, or patches. The particles are admixed under sterile conditions with a pharmaceutically acceptable carrier and any needed preservatives or buffers as may be required. Ophthalmic formulation, ear drops, and eye drops are also contemplated as being within the scope of this invention.

The ointments, pastes, creams, and gels may contain, in addition to the particles of this invention, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the particles of this invention, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the particles in a proper medium. Absorption enhancers can also be used to increase the flux of the compound across the skin. The rate can be controlled by either providing a rate controlling membrane or by dispersing the particles in a polymer matrix or gel.

In another aspect, a method of treating a disease in a subject is provided. In some embodiments, the method includes providing a polymeric core containing a pharmaceutically active agent; and conjugating an antibody or fragment thereof to the surface of the particle, wherein the antibody or fragment thereof targets an endogenous immune cell subset. In some embodiments, the method includes providing a polymeric core containing a pharmaceutically active agent; and conjugating an antibody fragment to the surface of the particle, wherein the antibody fragment targets an endogenous immune cell subset. In some embodiments, the endogenous immune cell subset is a T-cell. In some embodiments, the method includes targeting a T-cell to deliver pharmaceutical agents to specific T-cells for the treatment of proliferative disease. In some embodiments, the method includes targeting a T-cell to deliver pharmaceutical agents to cells in the tumor microenvironment or draining lymph node for the treatment of proliferative disease. In some embodiments, the endogenous immune cell subset is an MDSC. In some embodiments, the method includes providing a polymeric core containing a pharmaceutically active agent; and conjugating an antibody or fragment thereof to the surface of the particle, wherein the antibody or fragment thereof targets an MDSC. In some embodiments, the method includes providing a polymeric core containing a pharmaceutically active agent; and conjugating an antibody fragment to the surface of the particle, wherein the antibody fragment targets an MDSC. In some embodiments, the method includes targeting an MDSC to deliver pharmaceutical agents to specific an MDSC for the treatment of proliferative disease. In some embodiments, the method includes targeting a an MDSC to deliver pharmaceutical agents to cells in the tumor microenvironment or draining lymph node for the treatment of proliferative disease. In some embodiments, the method comprises administering the particle. In some embodiments, the method comprises administering the pharmaceutical composition to the subject. In some embodiments, the disease is an inflammatory disease or neoplastic disorder (e.g., cancer, benign neoplasm). In some embodiments, the disease is a proliferative disease. In some embodiments, the treated proliferative disease is cancer. In some embodiments, the cancer is melanoma. In some embodiments, the cancer is metastatic melanoma. In some embodiments, the cancer is colorectal cancer. In some embodiments, the cancer is metastatic colorectal cancer. In certain embodiments, the proliferative disease is an autoimmune disease. In some embodiments, the step of administering comprises administering the pharmaceutical composition parenterally. In some embodiments, the step of administering comprises administering the pharmaceutical composition orally. In certain embodiments, the step of administering comprises administering the pharmaceutical composition intravenously. In certain embodiments, the step of administering comprises administering the pharmaceutical composition intravenously and not intraperitoneally. In certain embodiments, the step of administering does not comprise administering the pharmaceutical composition via intraperitoneal injection. In some instances, the particle is used to deliver a prophylactic agent. In certain embodiments, the particle is used to deliver diagnostic agents, such as a contrast agent or labelled agent for imaging (e.g., CT, NMR, x-ray, ultrasound). The particle may be administered in any way known in the art of drug delivery, for example, intravenously, intramuscularly, subcutaneously, intradermally, transdermally, intrathecally, submucosally, sublingually, rectally, vaginally, etc.

Another aspect of the present disclosure relates to kits for use in preparing or administering the inventive particles or compositions thereof. A kit for forming particles may include a polymeric core and an antibody or fragment thereof or precursor thereof, as well as any solvents, solutions, buffer agents, acids, bases, salts, targeting moiety, etc. needed in the particle formation process. A kit for forming particles may include a polymeric core and an antibody fragment, as well as any solvents, solutions, buffer agents, acids, bases, salts, targeting moiety, etc. needed in the particle formation process. Different kits may be available for different targeting moieties. In certain embodiments, the kit includes materials or reagents for purifying, sizing, and/or characterizing the resulting particles. The kit may be useful in a method of the disclosure. The kit may also include instructions on how to use the materials in the kit. The one or more agents (e.g., pharmaceutically active agent) to be encapsulated in the particle are typically provided by the user of the kit.

Kits are also provided for using or administering the inventive particle or pharmaceutical compositions thereof. The particle may be provided in convenient dosage units for administration to a subject. The kit may include multiple dosage units. For example, the kit may include 1-100 dosage units. In certain embodiments, the kit includes a week supply of dosage units, or a month supply of dosage units. In certain embodiments, the kit includes an even longer supply of dosage units. The kits may also include devices for administering the particles or a pharmaceutical composition thereof. Exemplary devices include syringes, spoons, measuring devices, amongst others. The kit may optionally include instructions for administering the inventive particles (e.g., prescribing information).

In another aspect, the use of a particle to treat a proliferative disease in a subject is provided. In certain embodiments, the proliferative disease is cancer. In certain embodiments, the proliferative disease is an autoimmune disease. In certain embodiments, the particle comprises: a polymeric core containing a pharmaceutically active agent; and an antibody or fragment thereof conjugated to the surface of the particle, wherein the antibody or fragment thereof targets a T-cell. In certain embodiments, the particle comprises: a polymeric core containing a pharmaceutically active agent; and an antibody fragment conjugated to the surface of the particle, wherein the antibody fragment targets an endogenous immune cell subset. In some embodiments, the endogenous immune cell subset is a T-cell. In some embodiments, the endogenous immune cell subset is a myeloid-derived suppressor cell (MDSC). In certain embodiments, the particle comprises: a polymeric core containing a pharmaceutically active agent; and an antibody or fragment thereof conjugated to the surface of the particle, wherein the antibody or fragment thereof targets an MDSC. In certain embodiments, the particle comprises: a polymeric core containing a pharmaceutically active agent; and an antibody fragment conjugated to the surface of the particle, wherein the antibody fragment targets an MDSC.

The following examples are intended to illustrate certain embodiments of the present invention, but do not exemplify the full scope of the invention.

### EXAMPLES

These examples describe the characterization, drug-loading, and biological activity of poly(lactic-co-glycolic acid) (PLGA)-based nanoparticles (NP) and an antibody or fragment thereof conjugated to the surface of the nanoparticle, wherein the nanoparticle with a polymeric core contains a pharmaceutically active agent. The synthetic and biological examples described in this application are offered to illustrate the particles, pharmaceutical compositions, and methods provided herein and are not to be construed in any way as limiting their scope.

### Nanoparticle Formulation

PLGA-based nanoparticles were prepared using single-emulsion evaporation. PLGA (AP041, acid end-capped, 50:50, 10-15 kDa, Akina) was blended with Mal-PEG-PLGA (AI53, diblock copolymer, 50:50, 5-10 kDa, Akina) at 25% w/w. The polymers were dissolved in 1 mL dichloromethane (Sigma) and added to 6 mL of ice-cold 0.25% PVA (30,000-70,000 g/mol, Sigma) in 50 mM phosphate buffer, pH 5.8. The two phases were emulsified using a sonic probe (Qsonica Q700 with microtip, amplitude 10, 3 s power with 2 s break). SD-208-loaded nanoparticles were prepared by adding 10% (w/w) SD-208 (Selleckchem) to the solvent/polymer phase. The emulsion was stirred at room temperature for 3 h to evaporate the dichloromethane and afterwards purified by two wash-spin cycles in PBS at 20,000 g for 10 min. Nanoparticles s were assessed for size distribution using a Zetasizer Nano series ZS90, and drug encapsulation was determined by absorbance at 370 nm.

### Antibody Cleavage and Conjugation

IdeS and IdeZ (obtained from Genovis or Promega) were used for site-specific cleavage of full-length IgG antibodies into F(ab′)2 and Fc. IdeS was used for the anti-CD8 (BioXCell, YTS169.4), rat IgG2b isotype control (BioXCell, LTF-2), pembrolizumab (DFCI), human IgG4 isotype control (BioLegend, ET904), GITR (BioLegend, DTA-1), and Gr-1 (BioLegend, RB6-8C5). IdeZ was used for anti-PD-1 clone 332.6D2 from Dr. Gordon Freeman (DFCI) and mouse IgG2a isotype control (BioXCell, C1.18.4). Antibodies were diluted in PBS with 5 mM EDTA to 1-4 mg/mL and incubated for 1-2 h at the recommended concentration of 1 unit enzyme per μg of antibody at 37° C. Antibody cleavage was confirmed by non-reducing SDS PAGE. The antibody fragments were then reduced using 0.5 mM dithiothreitol (DTT, Sigma) for 30 min at 25° C. to retrieve free sulfhydryl groups for chemical linkage to the maleimide group on the nanoparticle surface. Free DTT was removed before conjugation using 7 kDa desalting columns (Thermo Scientific). Antibody concentration was measured by NanoDrop (Thermo Scientific), and 25 μg of antibody was added per 1 mg of polymer. The reaction was carried out for 2 h at 25° C. under shaking. The amount of antibody on the nanoparticle surface was quantified by BCA assay (Thermo Scientific). Western blot (following reducing SDS PAGE) was performed to confirm the absence of Fc on the nanoparticle surface using Fc- and F(ab′)-specific antibodies (Jackson ImmunoResearch, 112-035-008 and 112-035-006).

### Cell Culture

Murine T cells were enriched from spleens using the EasySep™ T cell enrichment kit (StemCell Technologies) and cultured in RPMI-1640 media supplemented with 10% FBS, 1% penicillin-streptomycin, 1% GlutaMAX™, 10 mM HEPES, 1 mM sodium pyruvate, and 55 nM 2-mercaptoethanol. B16-F10 (ATCC) were cultured in DMEM supplemented with 10% FBS and 1% penicillin-streptomycin, the media for ovalbumin-expressing B16 cells was further supplemented with 0.5 mg/mL geniticin. All supplements were obtained from Life Technologies.

Blood collars for human T cells were obtained from the Brigham and Women's Hospital Blood Donor Center. T cells were enriched using the Rosette Sep™ Human T cell enrichment kit, and cells were separated via ficoll gradient separation using SepMate™. T cells were cultured in ImmunoCult™-XF T Cell Expansion Medium supplemented with 10 ng/mL IL-2 (Peprotech) and activated with 25 μl/mL ImmunoCult™ Human T Cell Activator (all from StemCell Technologies). The purity of the isolated cells was determined using anti-human CD3 antibody (BioLegend) and confirmed to be greater than 95% purity.

### In Vitro T Cell Assays

In vitro binding of nanoparticles was assessed after incubation of 250,000 enriched T cells with fluorescently (DiD, Life Technologies) labelled nanoparticles at different concentrations for 30 min at 37° C. After the incubation, T cells were washed 3 to 5 times in PBS and directly assessed by flow cytometry for DiD fluorescence. T cells isolated from OT-I Rag^{-/-} mice were activated by Dynabeads Mouse T-Activator CD3/CD28 (Thermo Scientific) at a ratio of 2:1 T cell to bead or by ova-expressing B16 melanoma cells at a ratio of 10:1 T cell to B16 cell. Carboxyfluorescein succinimidyl ester (CFSE, BioLegend) or cell trace violet (CTV, Thermo Scientific) was used to assess T cell proliferation; the labeling was carried out according to the manufacturer's recommendations. Mouse TGFβ1 was purchased from Cell Signaling Technologies, and T cell supernatants were analyzed by mouse IFN-γ ELISA MAX™ (BioLegend).

### Flow Cytometry

The following antibody clones were used for assessments by flow cytometry (BD LSR Fortessa) using murine T cells: mCD8a 53-6.7, mCD8b YTS156.7.7, mCD4 GK1.5, mCD3e 145-2C11, mCD3 17A2, mPD-1 29F.1A12, mGranzyme B GB11, mCD45 30-F11, mCD62L MEL-14, mCD44 IM7, mGITR YGITR.765, mCD11b M1/70, mLy-6C HK1.4, and mLy-6G 1A8. The following clones were used for experiments involving human T cells: hPD1 EH12.2H7, hIFNy B27, hCD3 HIT3a. Zombie Aqua™ was used as a dead/live stain. All antibodies were purchased from BioLegend.

### Animal Experiments

Animal experiments were carried out according to protocols approved by Dana-Farber Cancer Institute, Institutional Animal Care and Use Committee (IACUC). Six-to-ten week-old C57BL/6 mice were purchased from Jackson Laboratory. For experiments designed to assess nanoparticle binding, 400,000 B16 melanoma cells were inoculated subcutaneously into the flanks of the mice. When the tumors had grown to ˜400 mm³ (tumor volume calculated as ½×length×width²), nanoparticles were administered intravenously. One or two hours later, tumors were cut into small pieces, and extracellular components were digested by addition of collagenase type IV (˜50 units/mL, Thermo Scientific) and DNase (˜20 units/mL, Roche). Tumor samples were homogenized using gentleMACS for 37 s. Red blood cells were removed by ACK buffer (Life Technologies) for all mouse tissue samples. For experiments designed to assess therapeutic efficacy, 200,000 MC38 cells were inoculated subcutaneously into the flanks of the mice. After 5 days, nanoparticles or free drugs were administered intravenously twice weekly up to a total of 7 injections. 2 mg of nanoparticles were administered, translating to a dose of 20 μg anti-PD-1 and 40 μg SD-208. For experiments designed to assess the ability to “warm” a tumor microenvironment, 200,000 MC38 cells were inoculated subcutaneously into the flanks of the mice. After 14 days, nanoparticles or free drugs were administered intravenously, and tumors were recovered 72 hours later. 2 mg of nanoparticles were administered, translating to a dose of 20 μg anti-PD-1 and 60 μg R848.

### Example 1

This example provides characterization of the nanoparticles, including the types of polymers used for the polymer core, percent of drug encapsulation, nanoparticle size and polydispersity index, as depicted in Table 1. The encapsulation efficiency is determined by the ratio of drug in particles compared to initial added drug prior to particle formation and purification.

TABLE 1
Polymer core and nanoparticle size, percent
encapsulation, and polydispersity index.

| | % Encapsu- | | Polydispersity |
|---|---|---|---|
| Polymer | lation | Size | index (PDI) |
| AP01-PLA 12.5 kDA | 67 | 302 nm | 0.16 |
| AP41-PLGA 50:50 12.5 kDa | 65 | 282 nm | 0.18 |
| AP45-PLGA 50:50 40 kDa | 57 | 291 nm | 0.21 |
| AP32-PLGA 75:25 30 kDa | 61 | 328 nm | 0.20 |

### Example 2

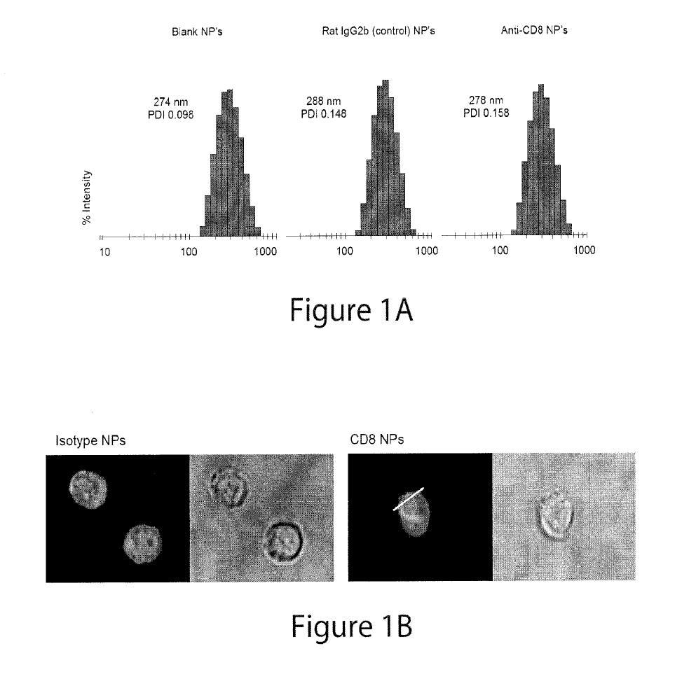

This example describes the in vitro characterization of the anti-CD8 nanoparticles (NP). FIG. 1A depicts the in vitro characterization of the anti-CD8 NP's, including the size distribution of optimized blank NP's, anti-CD8 NP's, and control formulations, and the PDI of each set of NP's.

### Example 3

Confocal microscopy of particle and CD8+ T-cell interaction was performed as follows. CD8+ T-cells were isolated from mouse spleens by negative selection, and the cytosol was stained with Carboxyfluorescein succinimidyl ester (CFSE). The isolated CD8+ T-cells were incubated with NP's labeled with the fluorescent dye DiIC18(5) (DiD), and conjugated to anti-CD8 antibody or isotype antibody control for 10 to 30 minutes in serum-free media. Unbound NP's were washed off by centrifugation at 300 g for 3 minutes. CD8+ T-cells with bound NP's on the cell surface were re-suspended in fresh media and confocal microscopy was performed to assess NP binding within 2 hours, using a spinning disk confocal microscope from Andor (Yokogawa CSU-X1). FIG. 1B provides confocal microscopy images of the CD8 and isotype NP's on the CD8+T-cell surface.

### Example 4

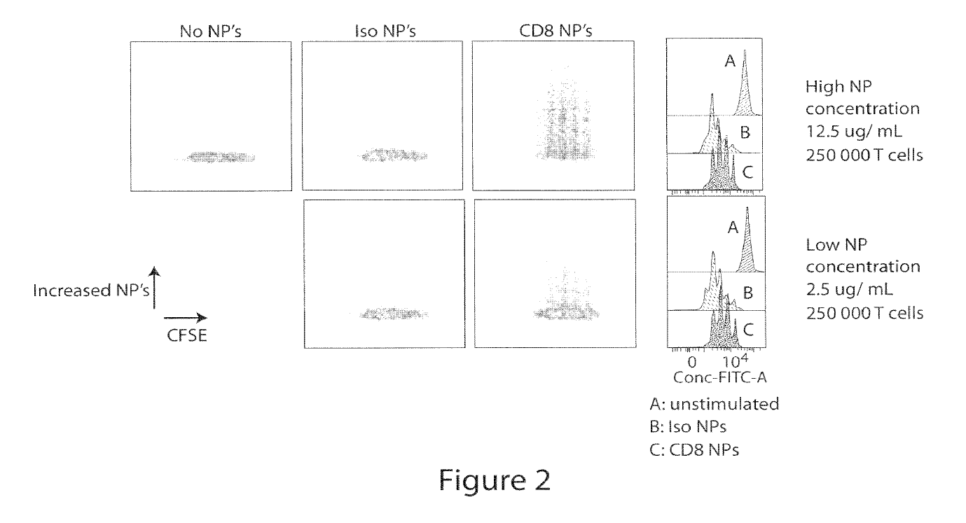

This example describes the activation of the CD8+T-cells by B16 tumor cells following CD8-NP binding. Ovalbumin-specific (OT-1) CD8+ T-cells were incubated with anti-CD8 NP's for 30 minutes, washed to remove unbound NP's, and co-cultured with ovalbumin (Ova-) expressing B16 tumor cells for 72 hours. Proliferation was assessed by CFSE dilution and NP binding by the fluorescent dye DiD that was loaded in the NP core, as depicted in FIG. 2.

### Example 5

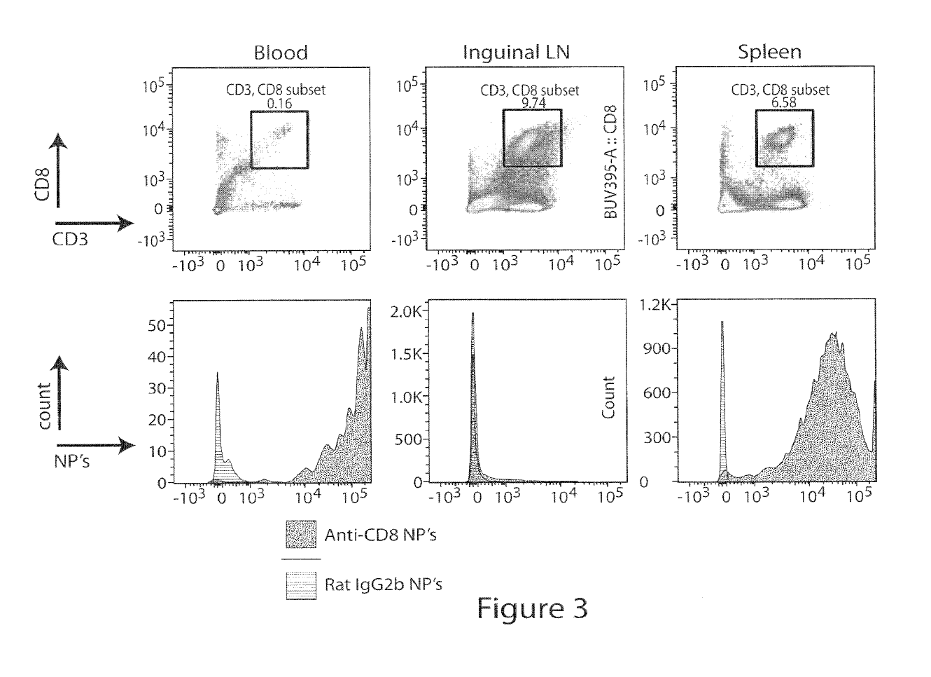

This example describes the binding of anti-CD8 NP's in vivo. DiD-labeled nanoparticles were injected intravenously, and detected on T-cells in blood, inguinal lymph nodes (LN) and spleen after 2 hours in circulation. FIG. 3 depicts the binding of anti-CD8 NP's in vivo.

### Example 6

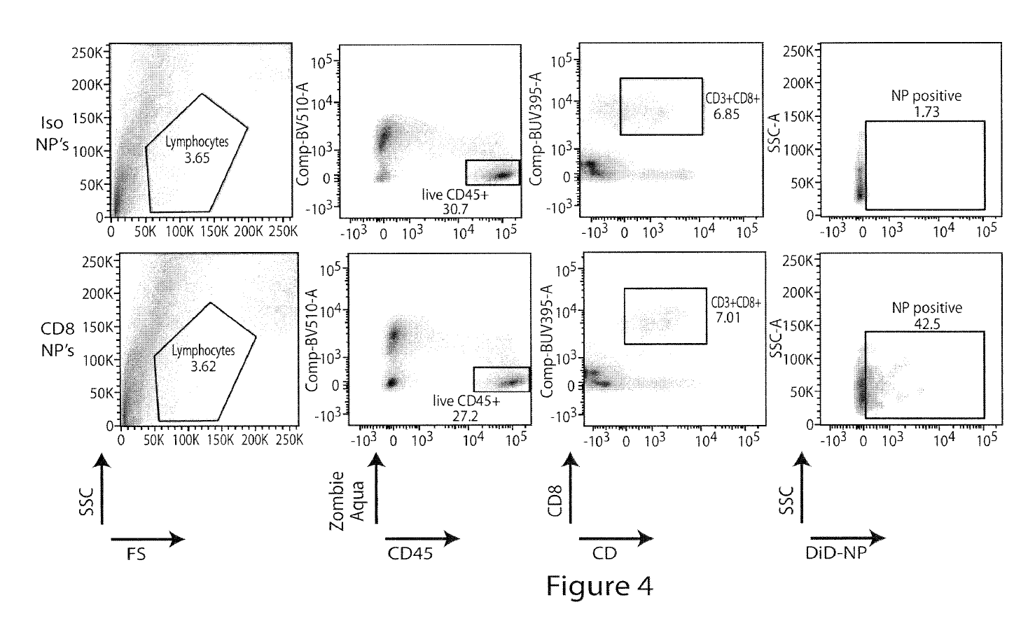

This example describes the binding of anti-CD8 NP's in tumor-bearing mice. B16 melanoma cells were injected subcutaneously in C57B6 mice, which developed tumors over 13 days to a size of ˜400 mm³. 1 mg of nanoparticles was injected intravenously and blood, tumor, tumor-draining lymph node and spleen were collected. FIG. 4 depicts the exemplified gating strategy on a tumor isolated after 24 hours.

### Example 7

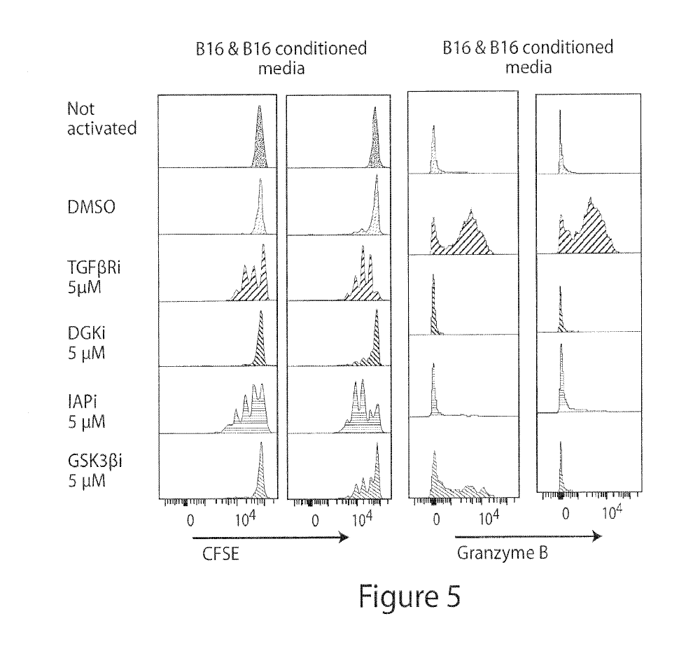

This example describes a small molecule inhibitor (SMI) screen, assessing the immunomodulatory effects for selected SMI's. The screened SMI's include: Transforming Growth Factor β receptor I kinase inhibitor (TGF-βRi), Diacylglycerol Kinase inhibitor (DGKi), Inhibitors of Apoptosis Proteins inhibitor (IAPi), and glycogen synthase kinase-3β inhibitor (GSK-3βi). Dentritic cells presenting SIINFEKL peptide were generated from bone marrow-derived cells and used to activate OT-I T cells in presence or absence of a tumor environment (B16 melanoma cells combined with conditioned media). Activation was performed for 72 hours in presence of different SMI's to assess the immunomodulatory effects of the SMI's. Enhanced proliferation was assessed by CFSE dilution, and intracellular staining was performed to assess Granzyme B production in T cells. FIG. 5 depicts the assessment of the effects of the SMI's on the enhanced proliferation and Granzyme B production in T cells.

### Example 8

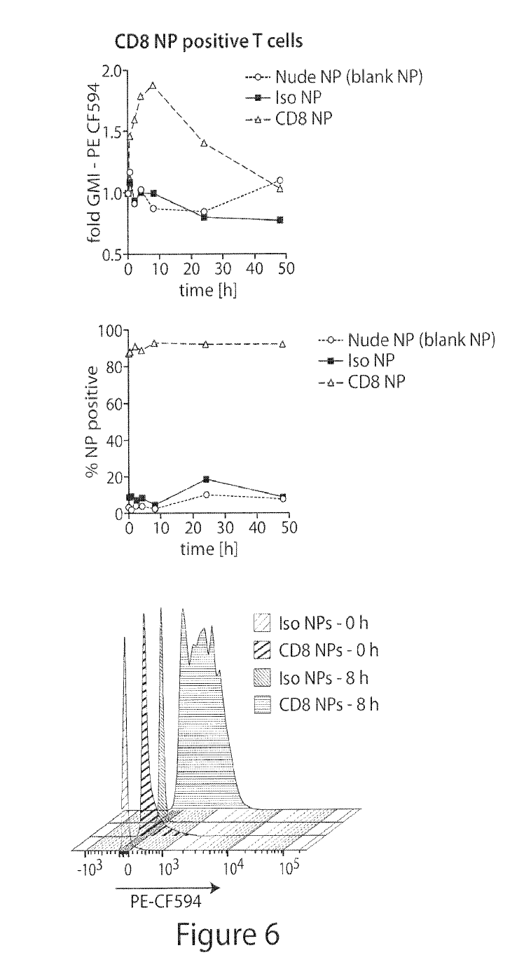

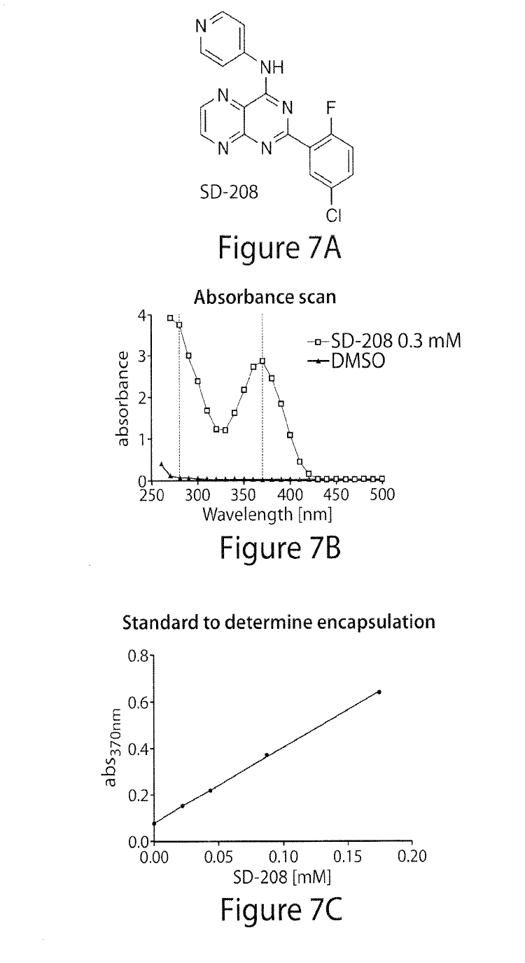

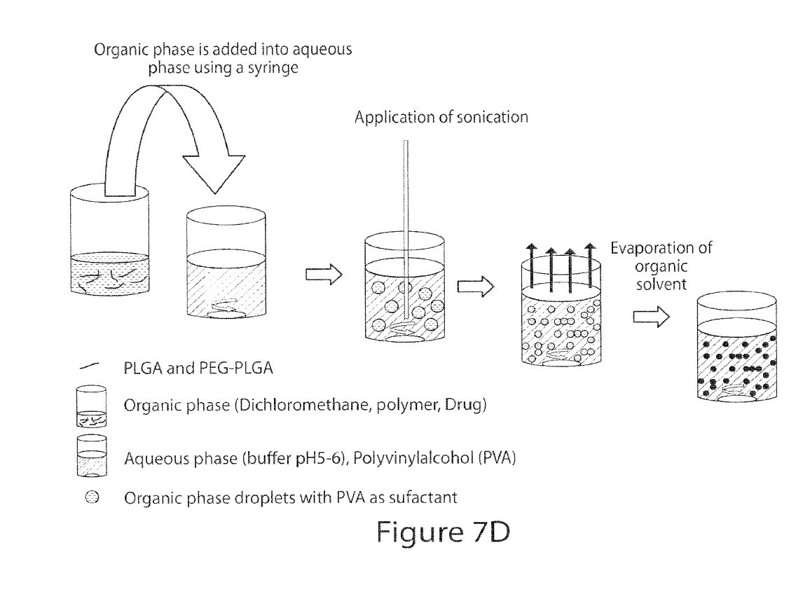

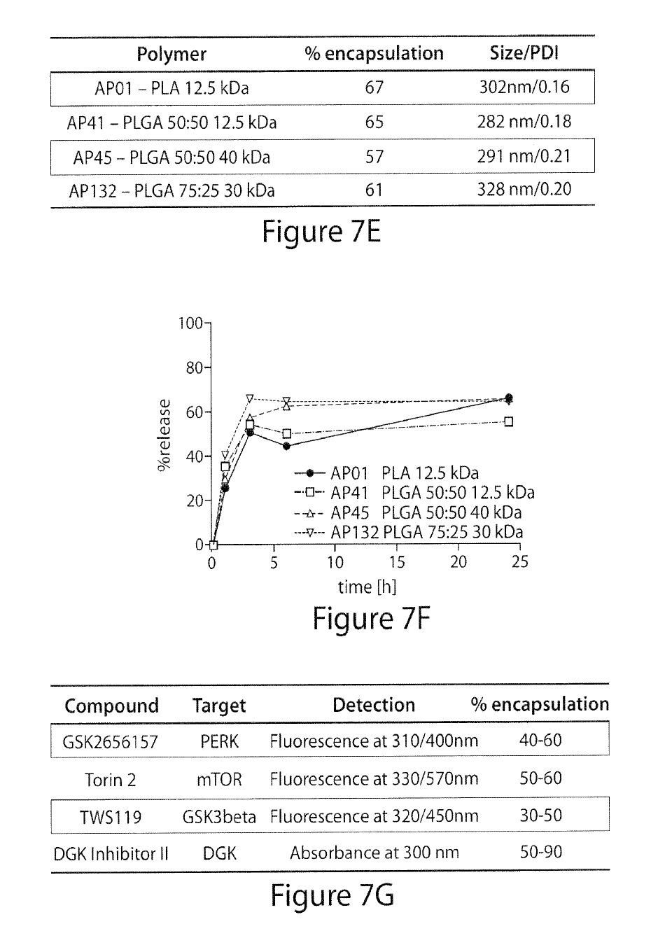

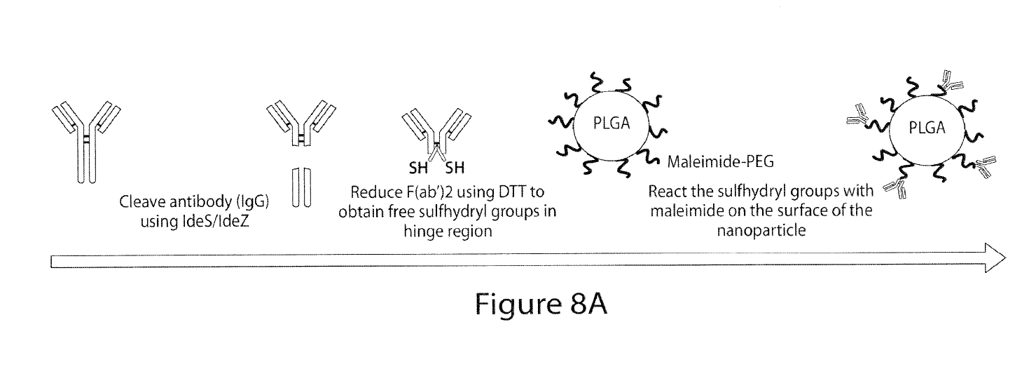

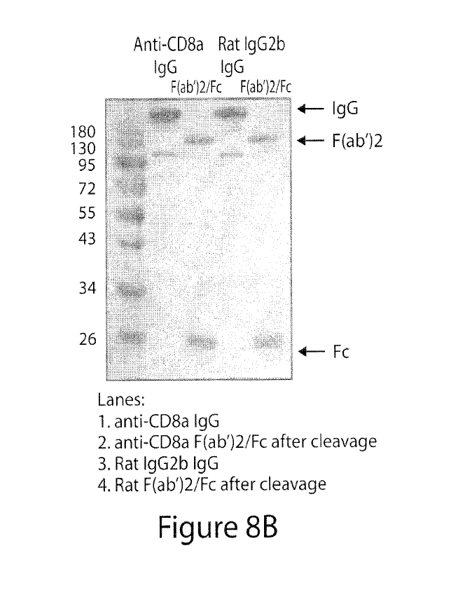

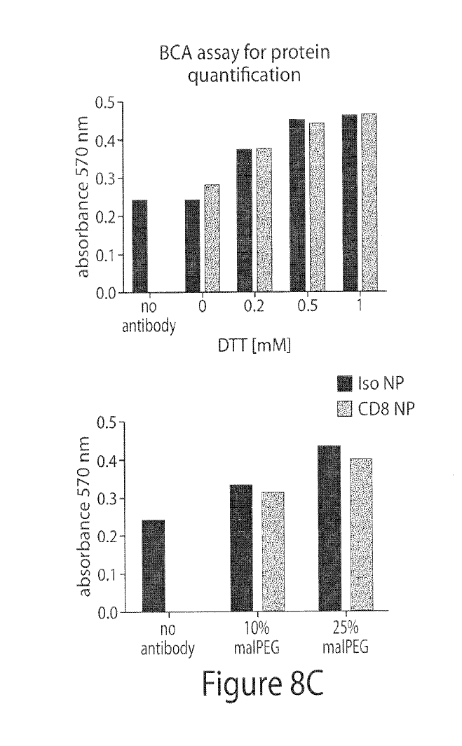

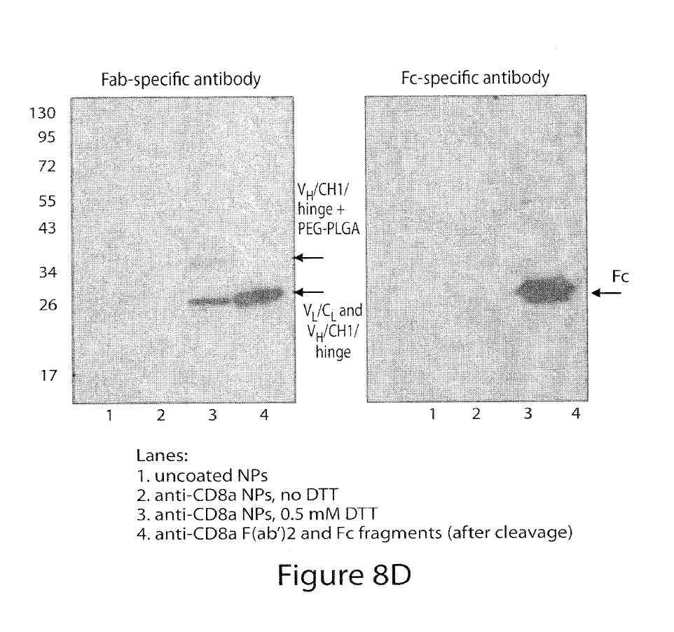

This example describes the internalization of CD8-targeted nanoparticles (NP) by CD8+ T-cells. F(ab′)2 conjugated and DiD-loaded nanoparticles were labeled using the pHAb Amine Reactive Dye (G9841, Promega), which has low fluorescence at pH greater than 7, but fluoresces at 532/560 nm in acidic solution (as found in lysosomal cell compartments). CD8+ positive T cells were incubated with isotype NP's and CD8-targeted NP's for the indicated time and the fluorescent signal was measured over time by flow cytometry. DiD was used to confirm nanoparticle binding, and the fluorescence intensity (of PE CF594) was used as a measure for NP internalization. FIG. 6 depicts the fluorescence intensity as a measure of the internalization of CD8-targeted nanoparticles by CD8+ T-cells.

### Generation of Manoparticles Targeting CD8⁺ T Cells

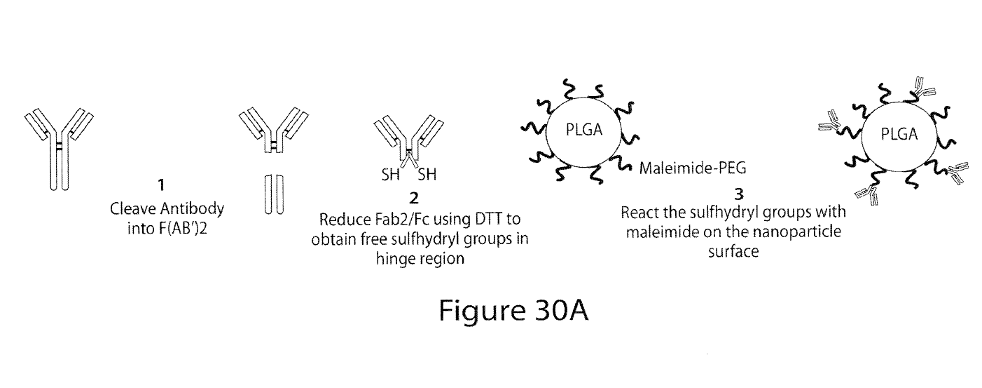

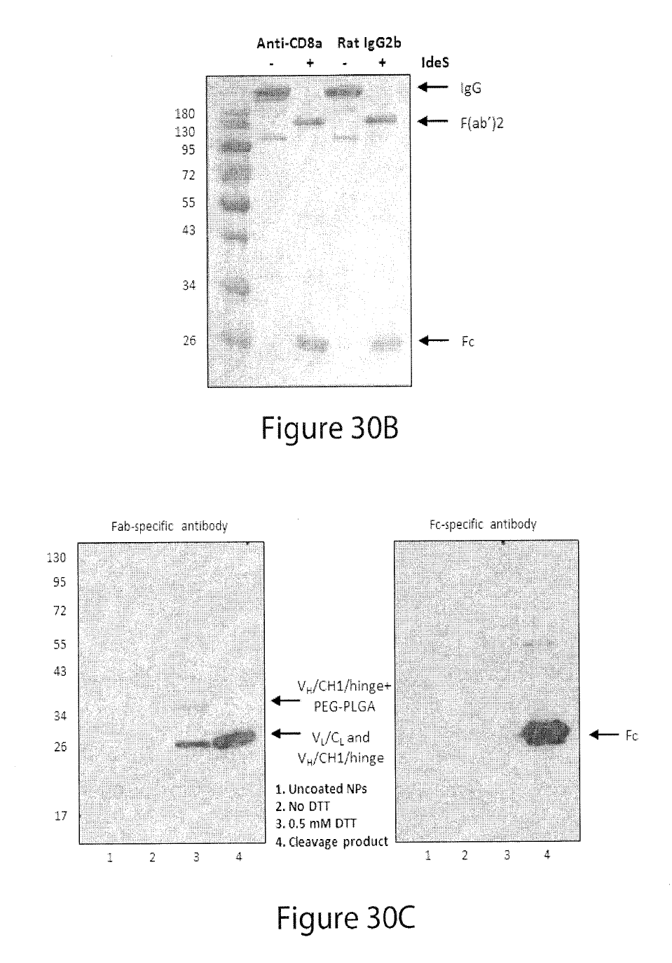

CD8+ T cell-specific nanoparticles were generated by conjugating anti-CD8a F(ab′)₂ fragments to the particle surface. These antibody fragments were produced by IdeS-mediated cleavage of full-length IgG molecules. High target affinity and avidity were thus achieved in the absence of potential interactions with Fc receptors expressed by phagocytic cells, which are a major means of nanoparticle clearance. Following the sequence-specific cleavage of the antibody below its hinge region, the disulfide bonds were reduced, and the resulting sulfhydryl groups were reacted with maleimide-functionalized PEGylated PLGA nanoparticles (scheme shown in FIG. 30A).

IdeS cleaved rat IgG2b antibodies (anti-CD8a and isotype control) with greater than 95% efficiency (FIG. 30B), and Western blot analysis confirmed that reduction of disulfide bonds (with 0.5 mM dithiothreitol) was required for conjugation of (Fab′)2 fragments (FIG. 30C, lanes 2 and 3 of left panel). Moreover, this analysis showed that the Fc portion that remained present in the reaction mixture as cleavage product was not conjugated to the nanoparticle surface (FIG. 30C, lane 3 of right panel compared to positive control in lane 4). The addition of F(ab′)2 did not lead to a significant increase in nanoparticle size (269±8 nm for Iso NPs and 273±8 nm for anti-CD8 NPs, n=8±SD) relative to uncoated nanoparticles (267±8 nm, n=9±SD), as determined by dynamic light scattering.

### Binding to CD8+ T Cells is Specific In Vitro and In Vivo

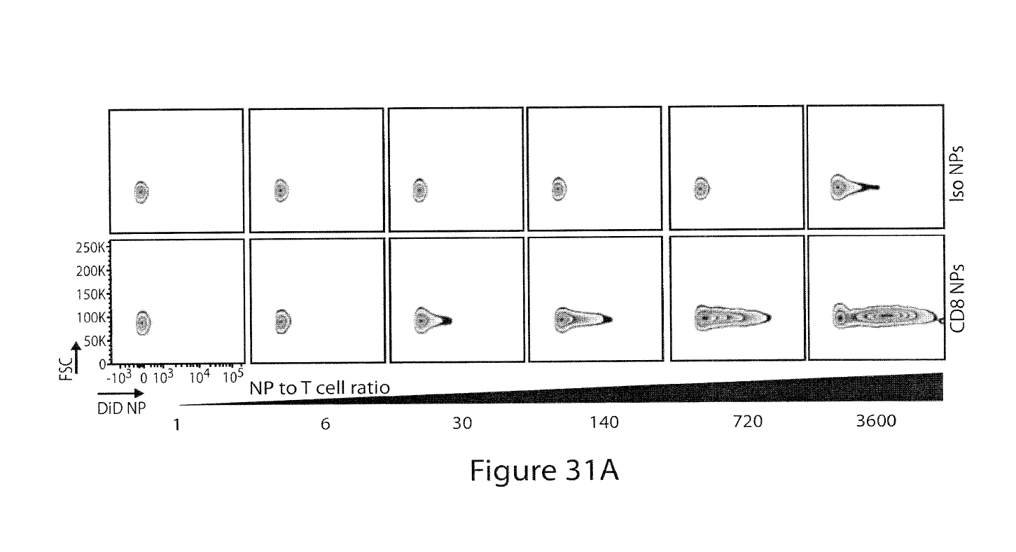

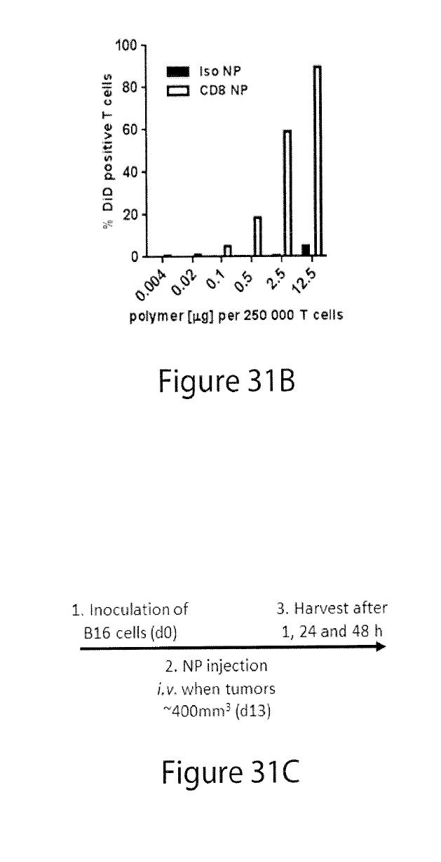

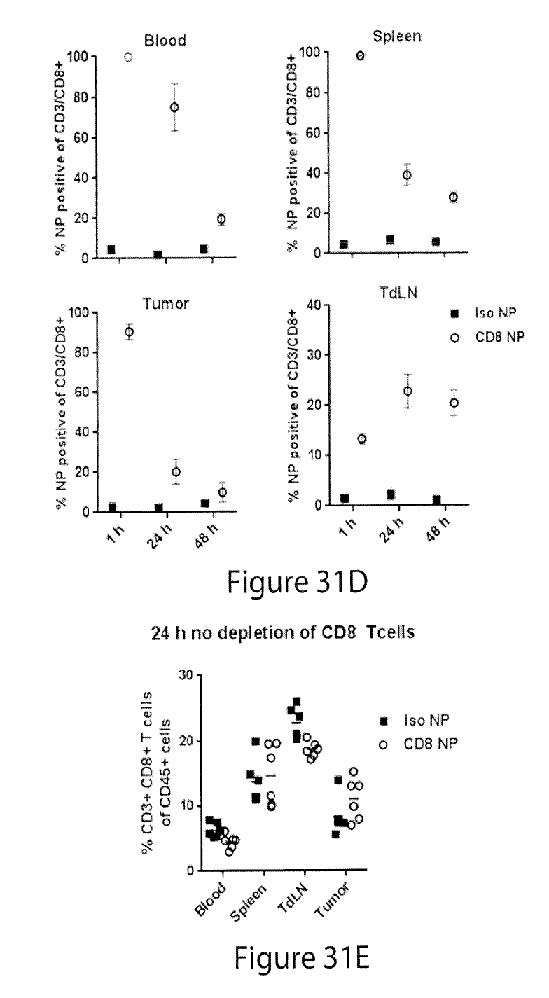

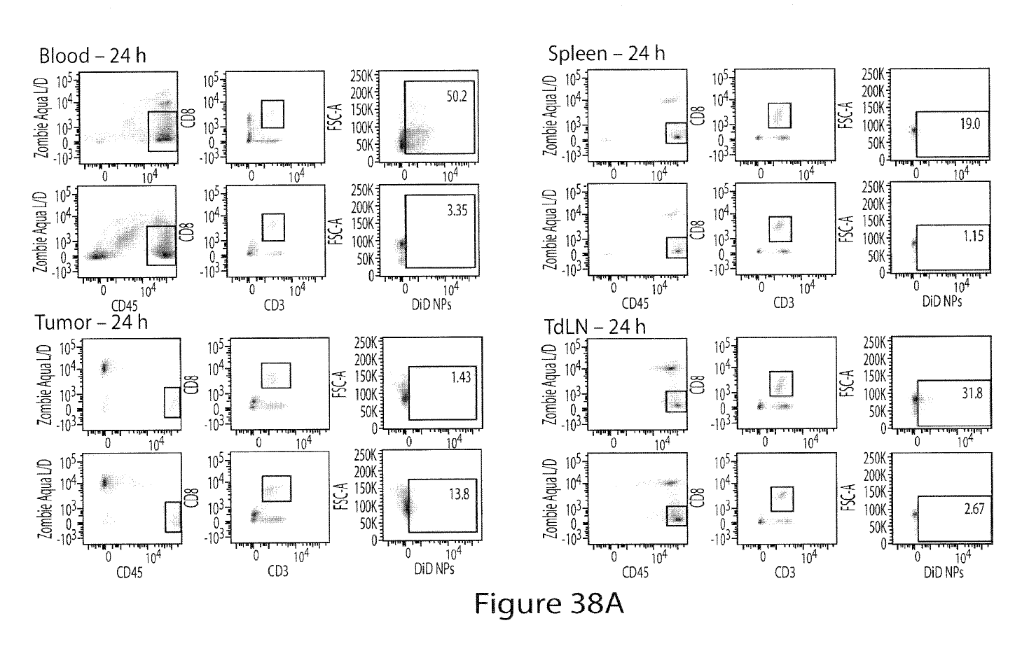

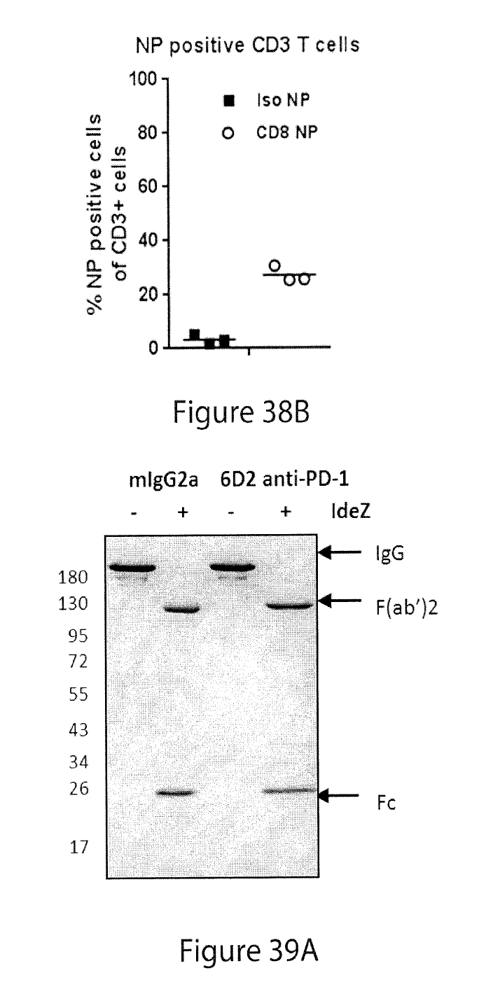

These CD8a-targeting nanoparticles bind to CD8 T cells, enriched from murine spleens, in a dose-dependent manner (FIG. 31A). At nanoparticle to T cell ratios greater than 3000:1, up to 90% of the T cell population were bound by CD8a-targeting nanoparticles with very little non-specific binding observed by isotype control nanoparticles (Iso NPs) (FIG. 31B). Ovalbumin-specific OT-I CD8+ T cells retain their ability to proliferate in the presence of ovalbumin-expres sing B16 melanoma cells when nanoparticles are bound to the surface of the T cells (FIG. 37). Next, we confirmed that an endogenous immune cell subset could be targeted in vivo. Nanoparticle binding was confirmed in a subcutaneous model of B16 melanoma. Mice with established tumors (˜400 mm³) were injected intravenously with CD8a-targeting nanoparticles, and immune cells were recovered from the circulation, spleen, tumor, and tumor-draining lymph node over a timeframe of 48 hours (FIG. 31C, gating strategy shown in FIG. 38A).

One hour after injection, 90-100% of the CD8⁺ T cells in the blood, spleen, and tumor tissue were bound by DiD-labeled CD8a-targeting nanoparticles, as determined by flow cytometry (FIG. 31D). Remarkably, CD8+ T cells isolated from the blood after one hour could not even be stained with free anti-CD8 antibody, evidently owing to steric shielding of the receptors by the nanoparticles. 27.2±2.4% of CD3⁺ T cells stained positively for DiD (FIG. 38B), which corresponds to the fraction of CD8⁺ T cells detected in the unbound Iso NP group, 26.6±5.8%. Hence, CD8a receptors on T cells in the blood are completely saturated by the CD8a-targeting nanoparticles after one hour. The percentage of CD8+ T cells recovered from blood, spleen, and tumor that are bound by CD8a-targeting nanoparticles decreases over 24 hours but persists for at least 48 hours.

Interestingly, the accumulation of CD8a-targeting nanoparticles in the tumor-draining lymph nodes increases over the time frame evaluated. It is possible that the nanoparticles accumulate passively in the draining lymph nodes and/or that T cells from the blood and/or tumor are trafficking there. Of note, unlike free anti-CD8a IgG, which results in target cell depletion owing to its isotype (14), administration of CD8a-targeting nanoparticles does not induce a significant reduction of CD8⁺ T cells (FIG. 31E). These data confirm that the Fc has been effectively removed during the cleavage and conjugation process.

Targeting to Functional Markers, such as PD-1, Can Also be Achieved

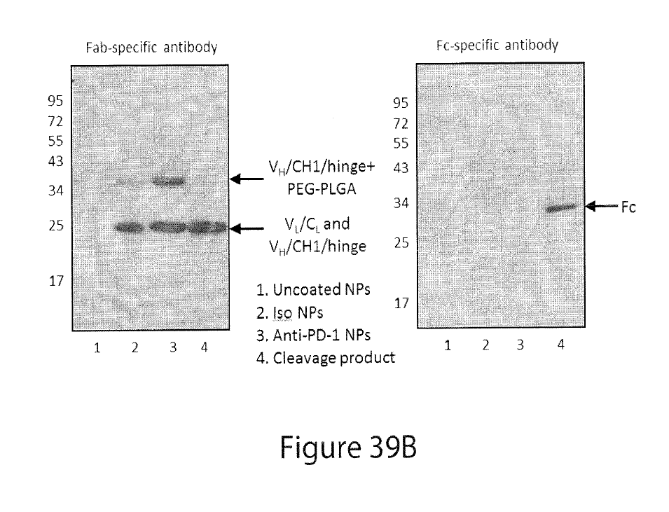

It has been shown that PD-1 identifies the tumor-reactive repertoire of CD8+ T cells that infiltrate human tumors (15) as well as neoantigen-specific CD8+ T cells in the peripheral blood of melanoma patients (16). We thus sought to target PD-1+ cells rather than all CD8+ cells. Anti-PD-1 clone 6D2 (mouse IgG2a, provided by Gordon Freeman) was cleaved using IdeZ (FIG. 39A), and the absence of Fc on the nanoparticle surface was again confirmed by Western blotting (FIG. 39B).

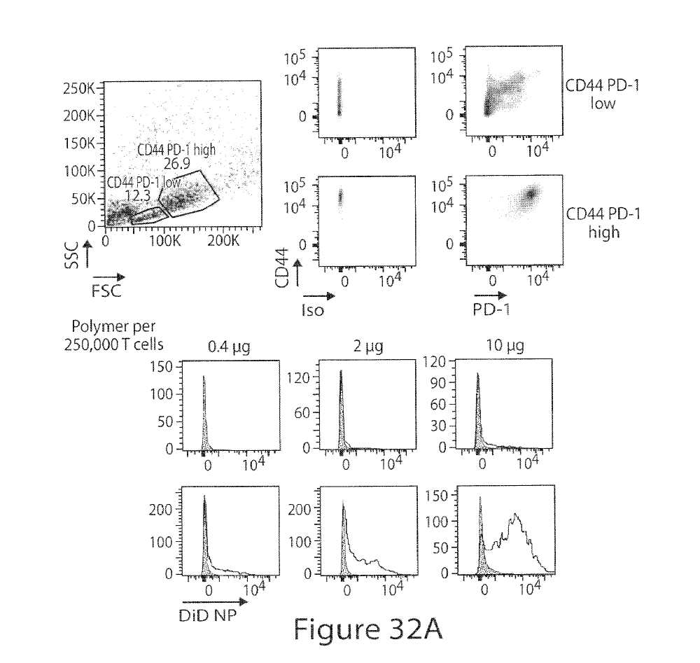

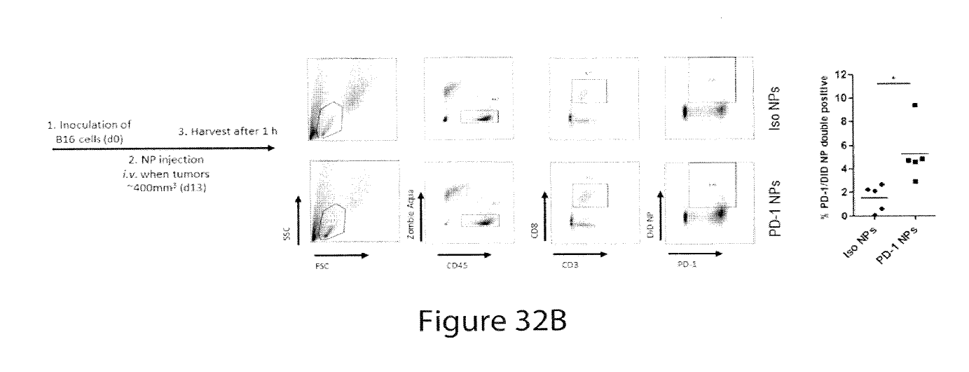

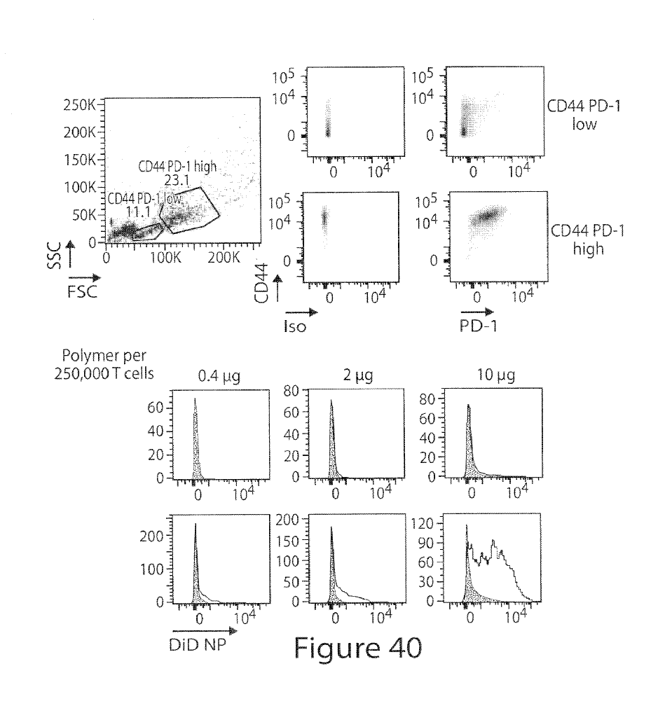

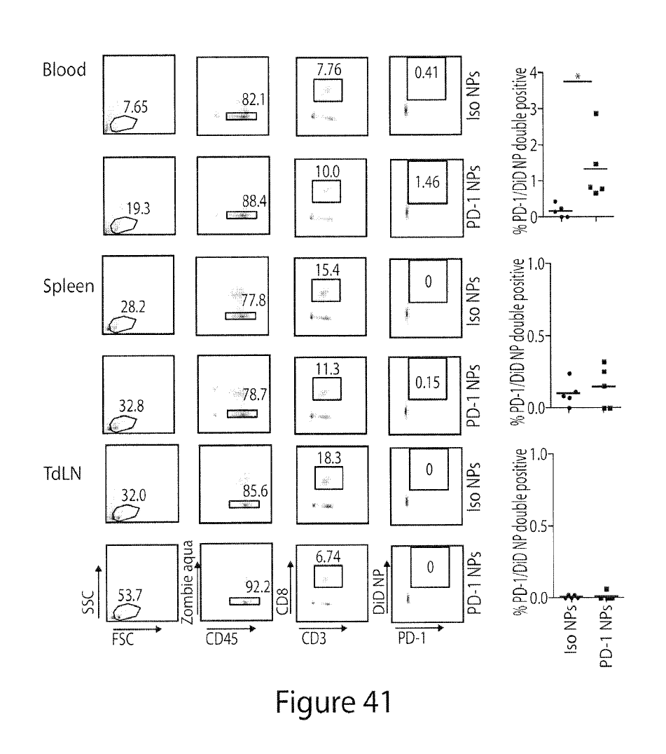

Naïve OT-I T cells were activated using ovalbumin-expressing B16 melanoma cells, and cells were gated according to their size and granularity. The smaller and less granular population exhibited lower expression levels of the activation markers CD44 and PD-1, and the binding of PD-1-targeting nanoparticles overlaid with isotype control nanoparticles for these cells (FIG. 32A). In contrast, the bigger and more granular population, which exhibited high expression levels of CD44 and PD-1, showed a dose-dependent increase in DiD signal with increasing amounts of anti-PD-1 nanoparticles. Similar results were obtained when the T cells were activated with anti-CD3/CD28 beads (FIG. 40).

To assess binding of PD-1-targeting nanoparticles in vivo, mice were inoculated with B16 melanoma cells, and nanoparticles were administered intravenously when the subcutaneous tumors reached a size of ˜400 mm³. Among immune cells isolated from tumor tissue that was harvested one hour after injection, ˜5% of PD-1+ T cells were also positive for anti-PD-1 nanoparticles, which was three-fold higher than the baseline observed for control isotype nanoparticles (FIG. 32B). We also found a significant increase (>10-fold) of nanoparticle-positive PD-1+ T cells in the blood, but this was not observed in the TdLN or spleen, where there were very few PD-1+ T cells at this time point (FIG. 41).

### Specific Binding to Human T Cells is Observed

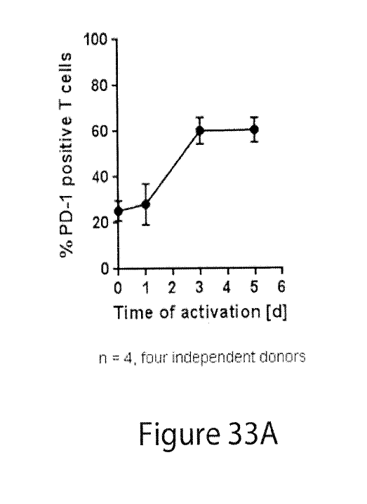

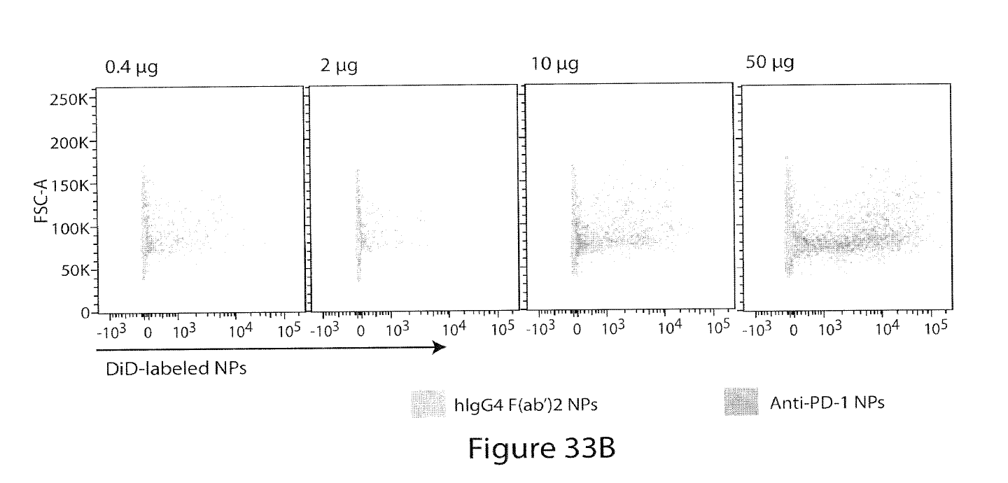

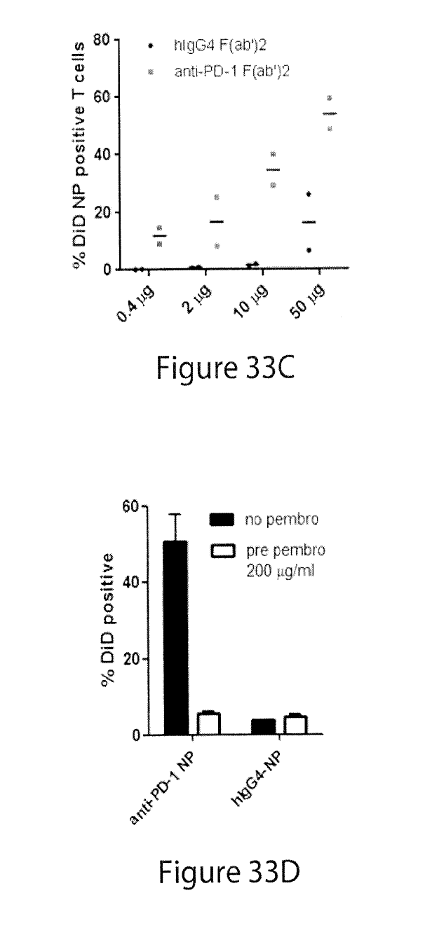

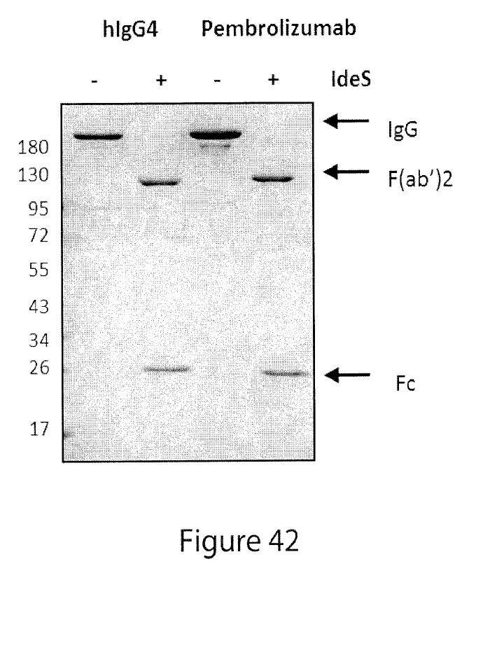

Pembrolizumab is a fully humanized anti-PD-1 antibody that is approved for the treatment of melanoma (17), non-small-cell lung cancer (18), and head and neck cancer (19). It was successfully cleaved (FIG. 42) and conjugated onto the surface of nanoparticles to assess the potential application of this platform for clinical use. Primary T cells were isolated from healthy human donors, and PD-1 expression was assessed by flow cytometry following activation with anti-CD3/CD28 complexes. PD-1 expression on human T cells increased to 60% by day three (FIG. 33A). As no further increase was observed by day five, T cells activated for three days were used for further binding studies using fluorescent nanoparticles. Pembrolizumab-coated nanoparticles showed dose-dependent binding to human T cells (FIG. 33B), with up to 60% of the cells being positive for DiD (FIG. 33C). This binding was prevented by pre-incubation of the activated T cells with free pembrolizumab (FIG. 33D), demonstrating that the binding was specific.

TGFβR1 Inhibitor Released from Nanoparticles Phenocopies Free Inhibitor

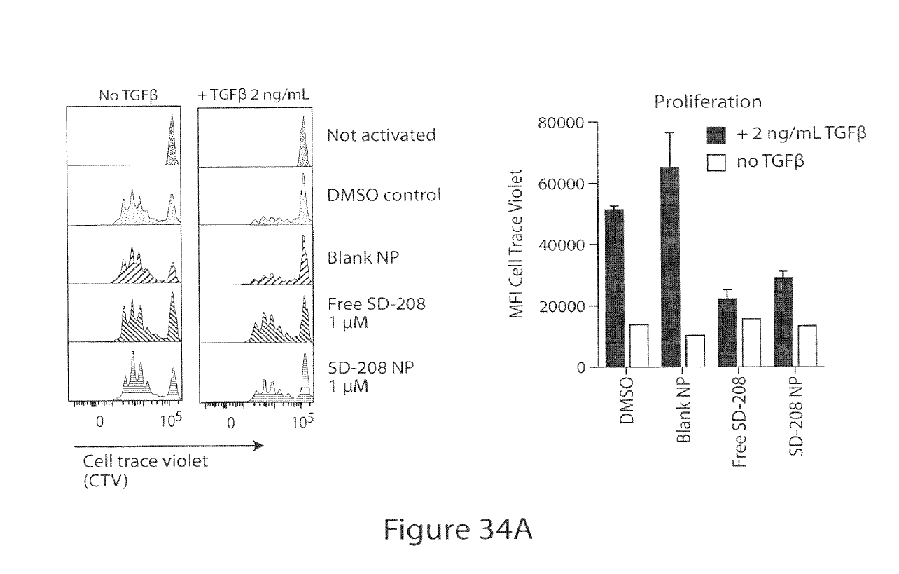

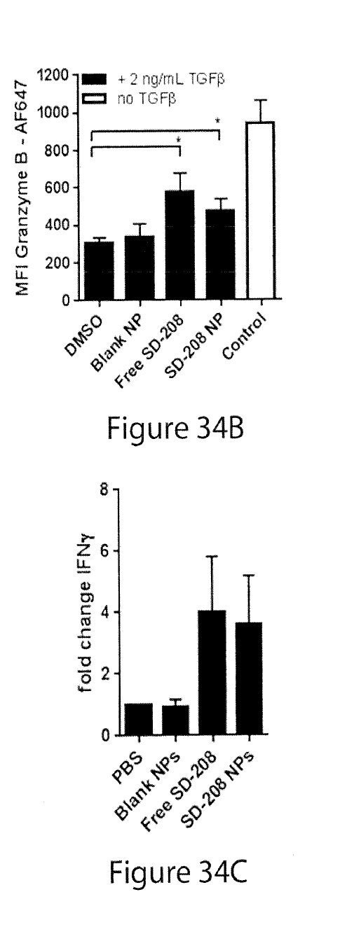

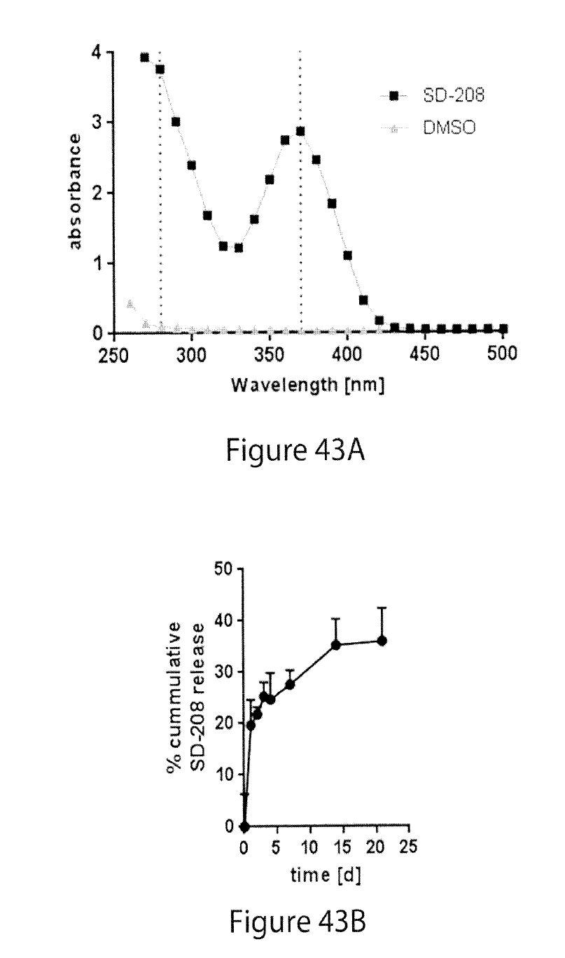

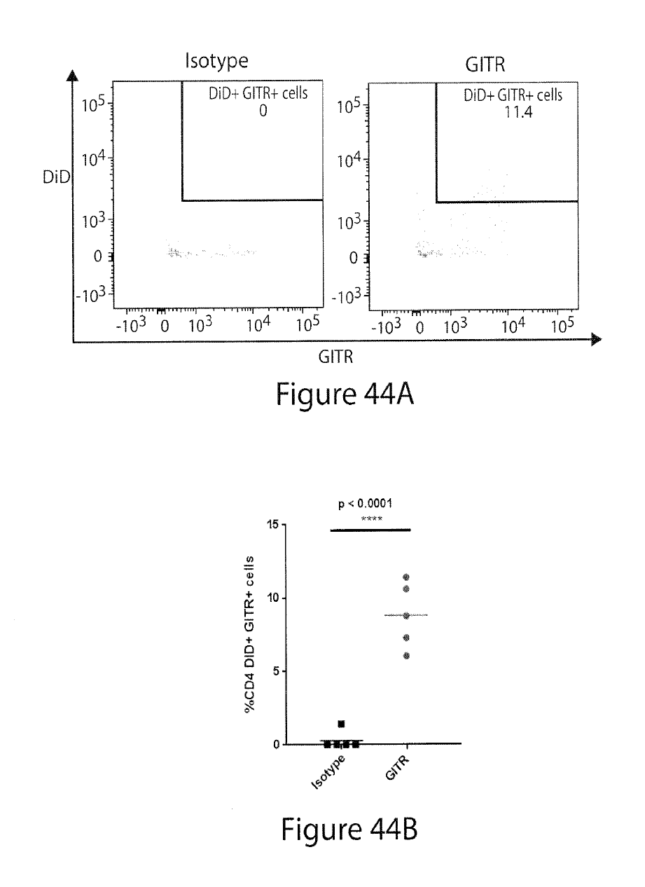

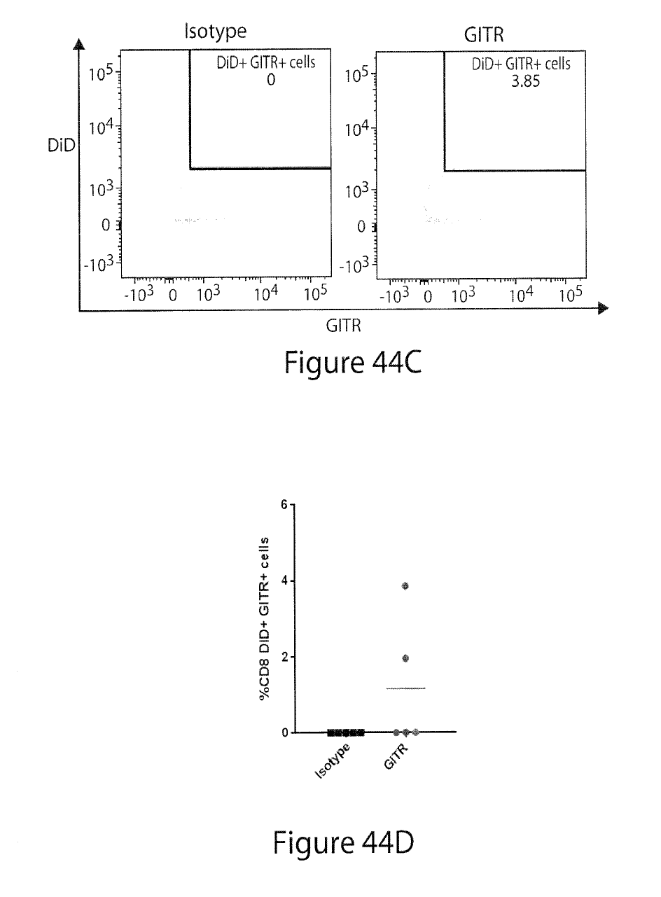

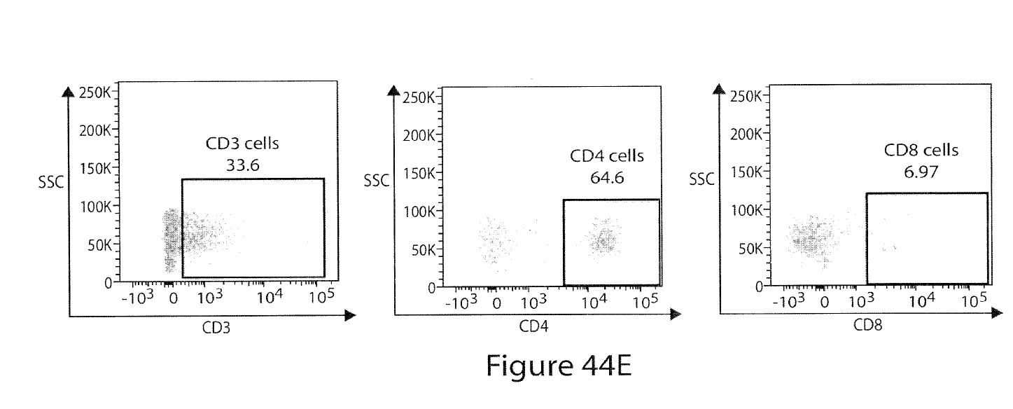

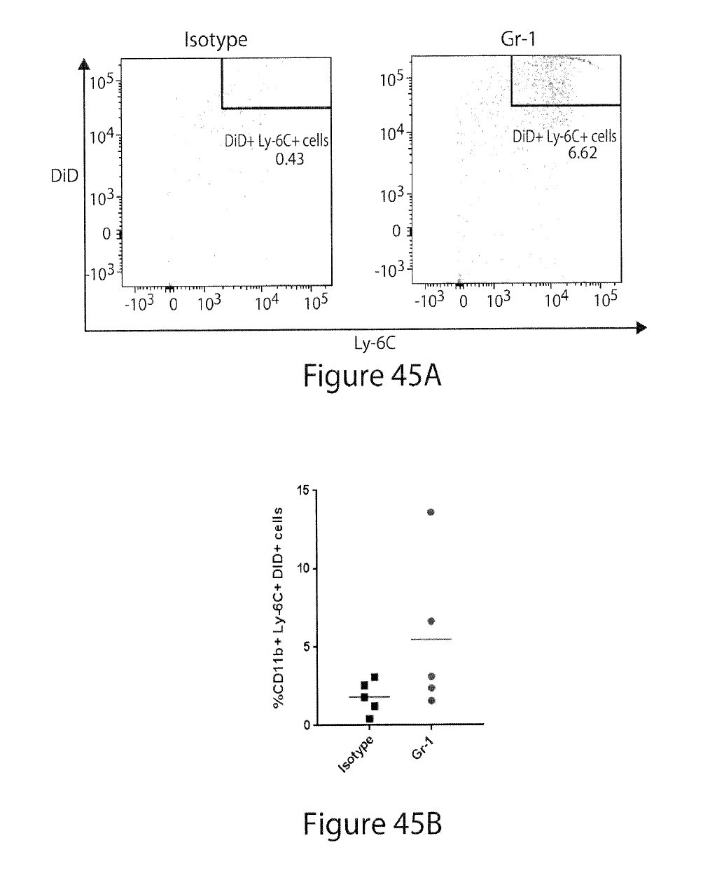

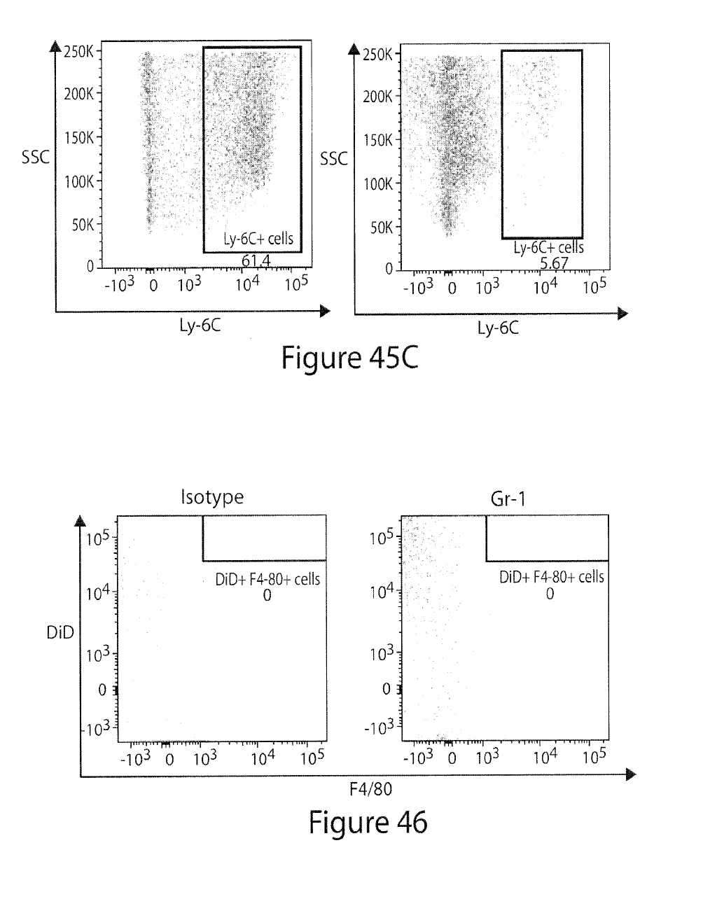

Having established that the nanoparticles can bind specifically to a defined target in vitro and in vivo, we sought to investigate the impact of targeting delivery of an immunomodulatory small molecule. SD-208 is an inhibitor of TGFβRI kinase (20) and thereby blocks immunosuppressive pathways induced by TGFβ, which is frequently expressed in tumor tissue (4). SD-208 is poorly water soluble and is therefore readily entrapped in the hydrophobic core of PEG-PLGA nanoparticles (20 μg/mg polymer). Encapsulation efficiency and drug release kinetics were analyzed by its absorbance maximum at 370 nm (FIG. 43A). Owing to its limited solubility in aqueous solution, SD-208 is released slowly from the nanoparticles over the course of weeks, as assessed in PBS containing 10% serum (FIG. 43B). SD-208 that was released from nanoparticles conferred similar effects to free SD-208 in cellular assays. Specifically, TGFβ-mediated inhibition of T cell proliferation was reversed in a comparable manner as shown by CFSE dilution (left panel, FIG. 34A) and its mean fluorescence intensity (right panel, FIG. 34A). Moreover, the markers of T cell function granzyme B and interferon γ (IFNγ) were upregulated to a similar extent as free inhibitor in DMSO (FIG. 34B and 34C).

### Therapeutic Efficacy is Observed Only if Delivery of Inhibitor is Targeted

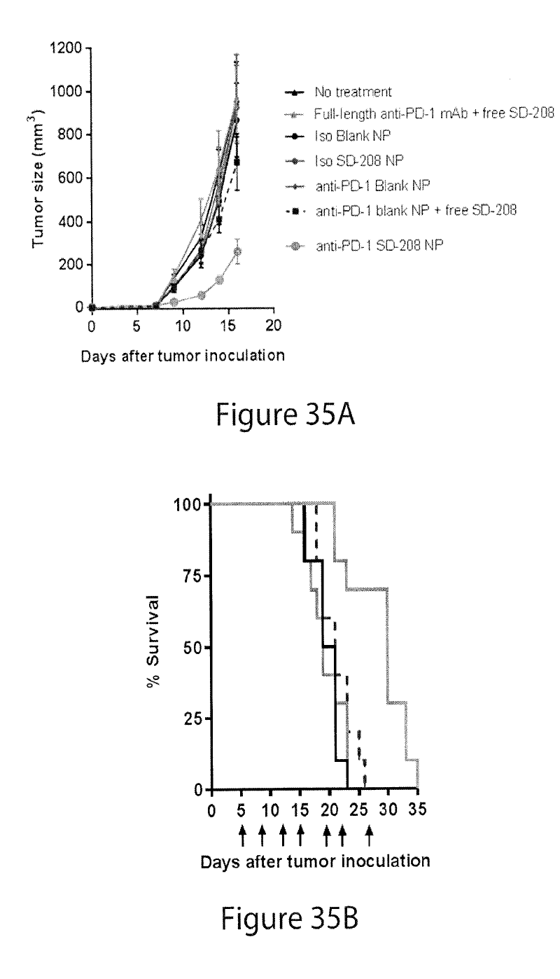

Because antitumor immune responses are highly dynamic and coordinated, we transitioned to in vivo studies using the MC38 model of colorectal cancer in order to assess for therapeutic efficacy. Growth of MC38 tumors is delayed by anti-PD-1 monotherapy at relatively high doses (300 μg/dose) (21). We sought to demonstrate that this platform can improve the therapeutic index and achieve efficacy at lower doses, thereby decreasing potential side effects, which remain a challenge in immunotherapy, particularly when multiple agents are being administered. Mice were inoculated with subcutaneous MC38 tumors and, beginning five days later, were administered anti-PD-1 and SD-208 intravenously at a dose of 20 μg anti-PD-1 and 40 μg SD-208, respectively Like all of the negative controls, free anti-PD-1 and SD-208 had no effect on tumor growth (FIG. 35A) or mouse survival (FIG. 35B). Delayed tumor growth and extended mouse survival were observed if and only if SD-208 was delivered by the PD-1-targeting nanoparticles. In contrast, PD-1-targeting nanoparticles administered in combination with free SD-208 had no impact, suggesting that targeted delivery of the small molecule drug was required. In this model, immune evasion ultimately prevailed, as the tumors eventually progressed. Though we successfully demonstrate he ability to focus the action of a TGFβR1 inhibitor on the tumor microenvironment, inhibition of TGFβ signaling may not be particularly relevant to this model or may not be sufficient to produce curative outcomes.

Targeting Delivery of R848 can Convert “Cold” Tumors into “Hot” Ones

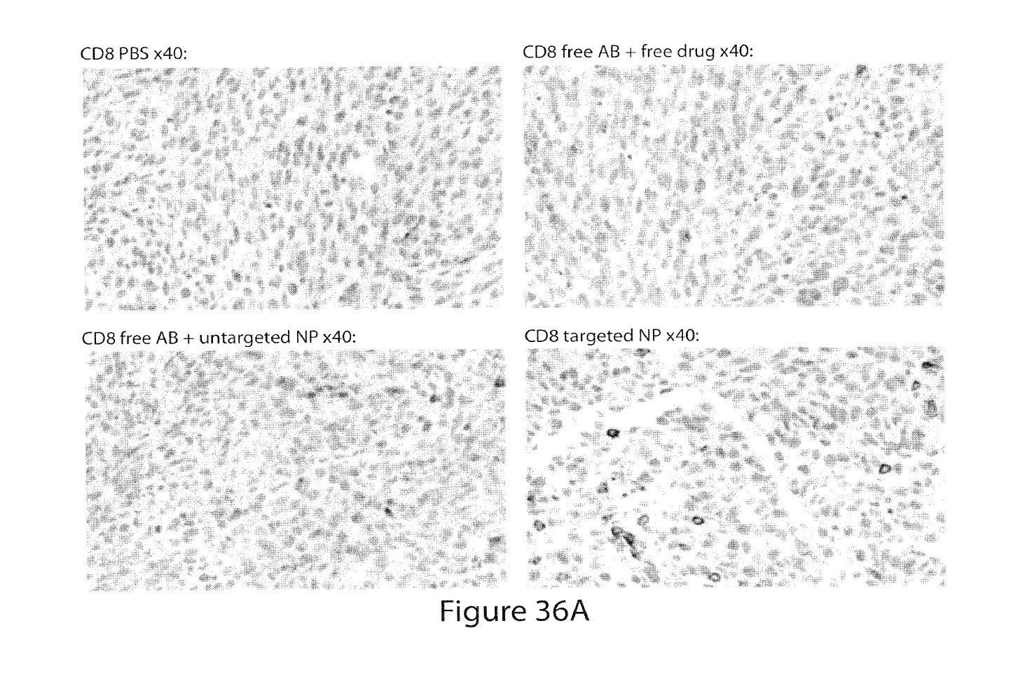

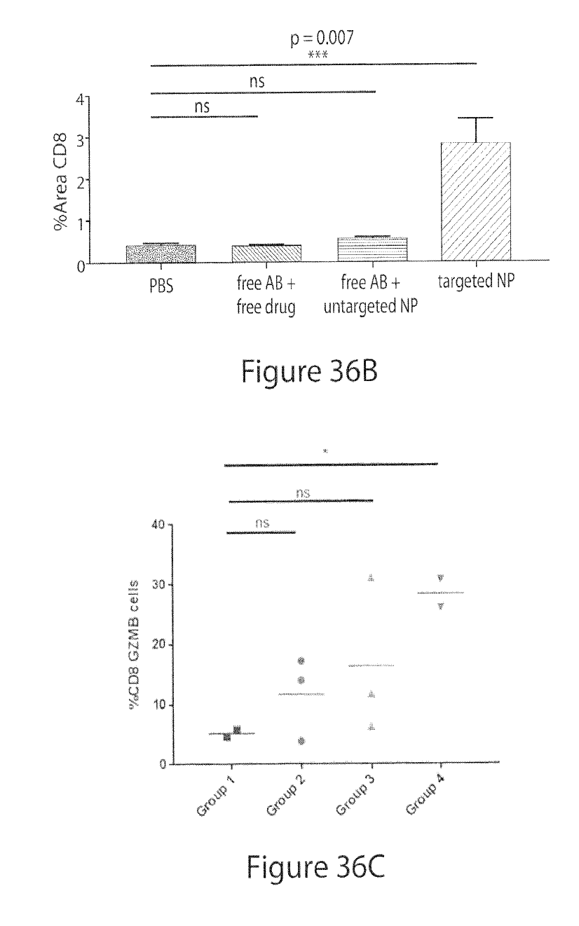

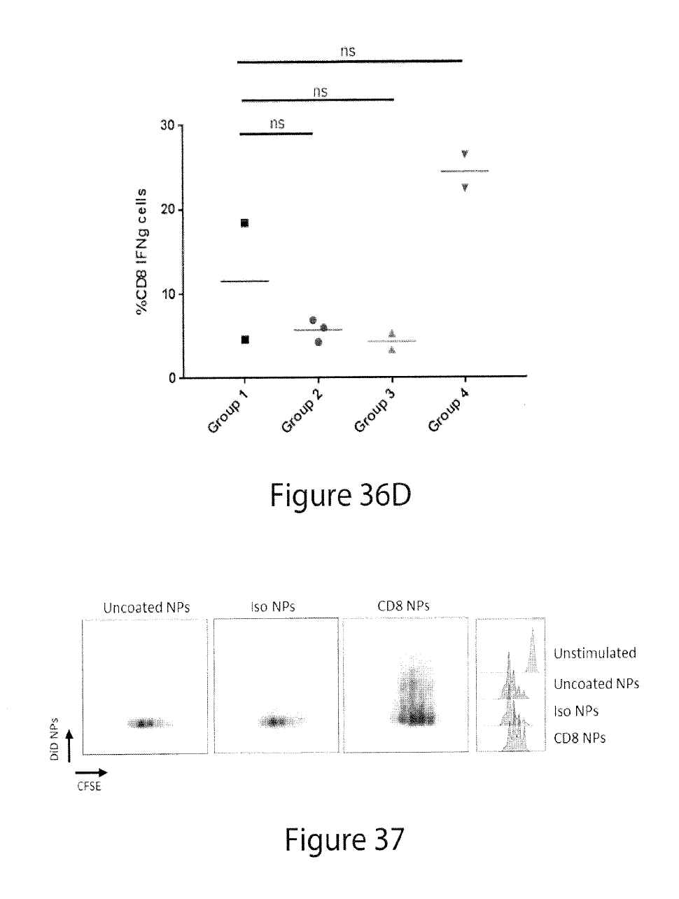

The majority of cancer patients still do not respond to immunotherapy, and a major obstacle is the fact that many tumors are not inflamed (22). Delivery of inhibitors of immunosuppression—including inhibitors of TGFβ, IDO, and PD-L1—would not be expected to have much impact in the absence of tumor-infiltrating lymphocytes (TILs). The possibility of inflaming a cold tumor microenvironment by leveraging the few PD-1+ cells that enter the tumors to deliver a Toll-like receptor 7/8 agonist, R848 was considered. (23). Delivery of R848 loaded in PD-1-targeting nanoparticles results in an increase in CD8+ T cells, as determined by immunohistochemistry (FIG. 36A,B). Functionally, these CD8+ T cells produced elevated levels of granzyme B and IFN-γ (FIG. 36C,D), as determined by flow cytometry. Again, the effect was specific to targeted delivery of the payload to PD-1-expressing cells. Delivery of free antibody and free small molecule had no effect, nor did delivery of free anti-PD-1 in combination with R848 loaded in untargeted particles, indicating that the nanoparticles do not passively accumulate in the tumors.

### Discussion

Unlike traditional cancer therapies, the immune system is adaptive and has capacity for memory. Adaptation is critical because cytotoxic agents select for resistant cancer clones, as tumors are heterogeneous and evolving (24). Memory is vital to achieving durable responses by preventing the recurrence that claims so many lives. Cancer immunotherapy can generate a coordinated and proliferative response that is relevant across numerous cancer types and their underlying mutations (25). Still, the fraction of patients who benefit from immunotherapy remains low, so new approaches that increase the therapeutic index are required.

The T cell-targeting nanoparticles described herein can concentrate immunomodulatory drugs at the site of immunosuppression following systemic administration. Whereas nanoparticles carrying cytotoxic payloads experience impaired diffusion into tumors (26), T cells can penetrate deeply into the tumor parenchyma. Moreover, leukocytes are the first items that nanoparticles contact upon intravenous injection. As such, it is much more likely a targeting nanoparticle will bind to a receptor on an immune cell than to a receptor on a distant cancer cell that may be secluded behind dense extracellular matrix and high interstitial fluid pressure. Still, targeting of nanoparticles to P-selectin, which is expressed on stromal endothelial cells in addition to cancer cells, vastly improves the efficacy of cytotoxic agents relative to administration of free drug (27), suggesting that targeting tumor vasculature may be a viable strategy as well.

Targeting of immune cells in vivo remains a nascent endeavor, particularly for delivery of small molecules. A previous study demonstrated that pre-incubation of LIF-containing particles targeted to CD4 with splenocytes in vitro prior to adoptive cell transfer supported expansion of Foxp3+ regulatory T cells (Tregs) as well as allograft survival (28). Such nanoparticles could be administered intraperitoneally to increase the percentage of Tregs in lymphoid compartments (29), though untargeted control particles were not included for comparison in either study. It is possible that Treg development could be induced by administration of free TGFβ and IL-2 or by sustained release of these two biologics from nanoparticles even in the absence of targeting to CD4 cells. The data presented herein are the first to show targeted delivery of an immunomodulatory small molecule to endogenous immune cell subsets in vivo following intravenous administration.

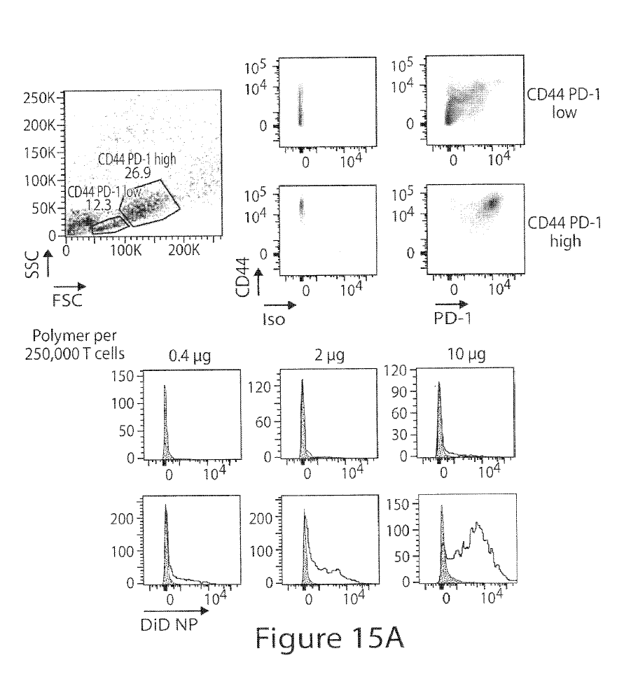

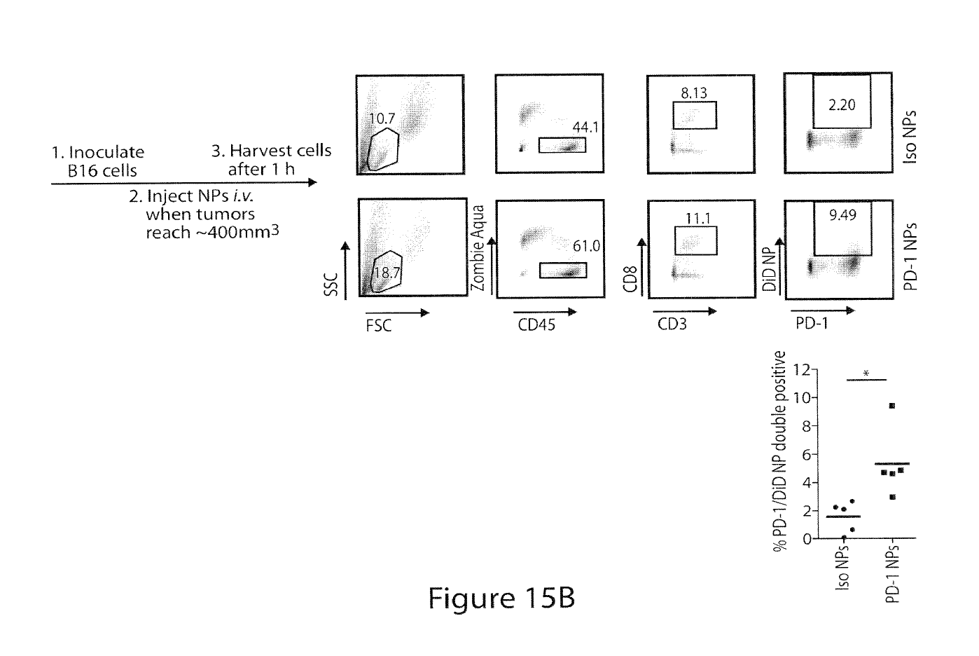

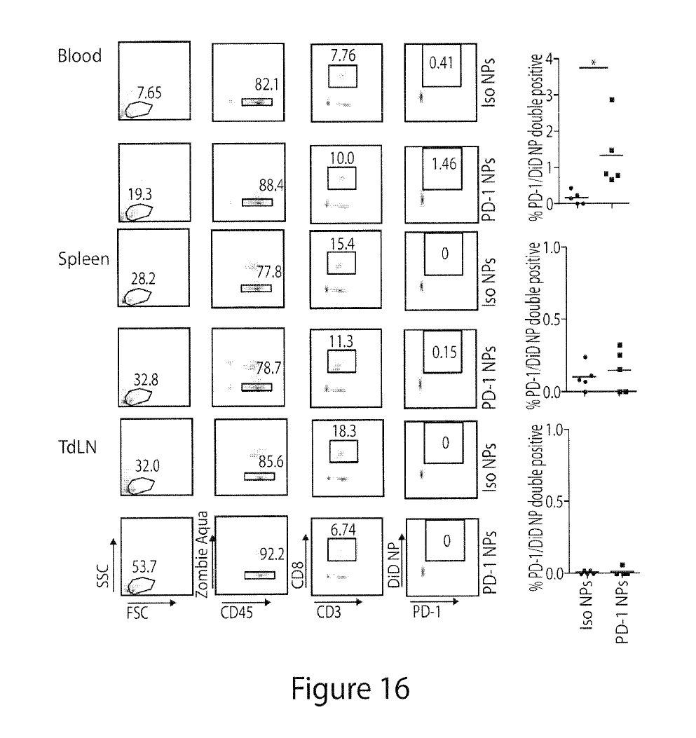

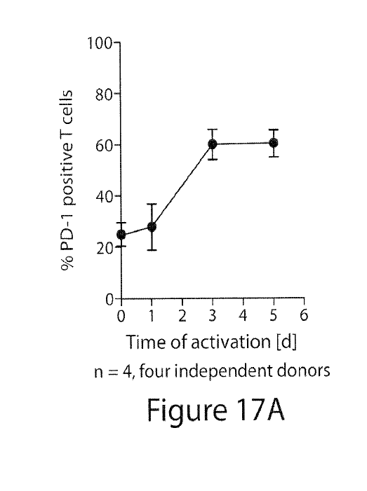

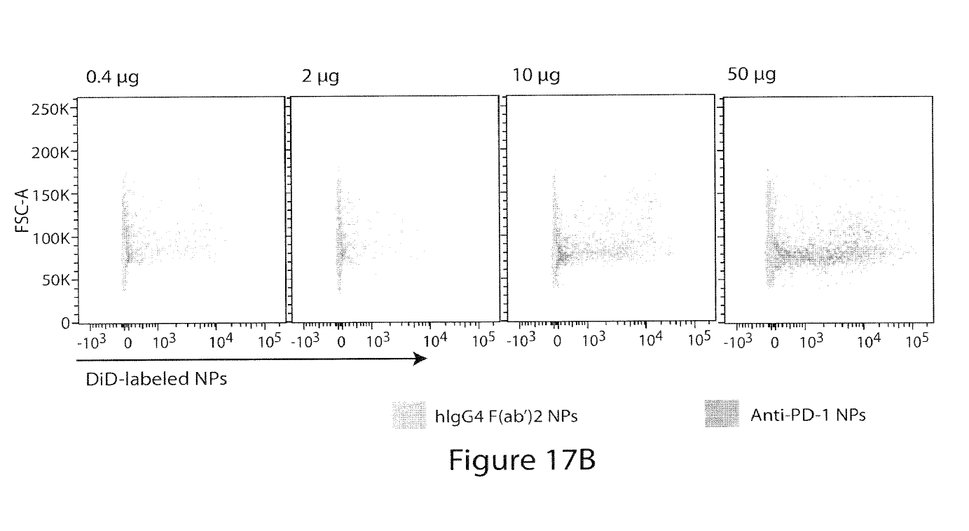

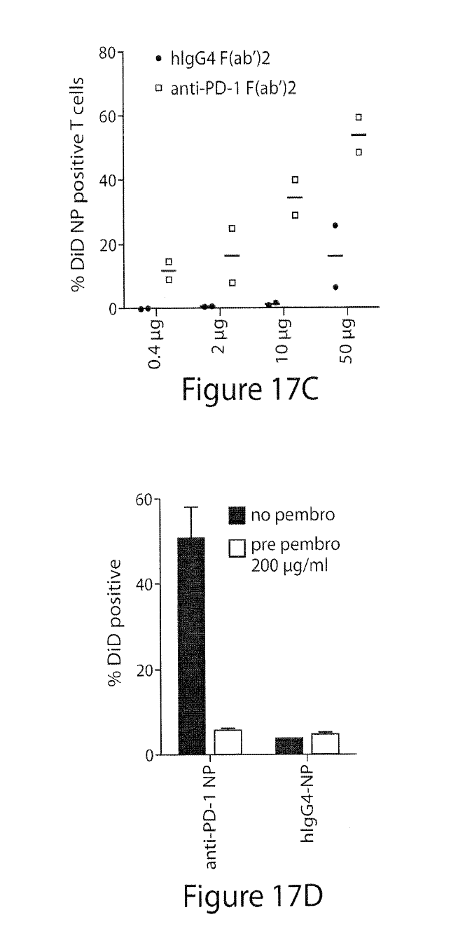

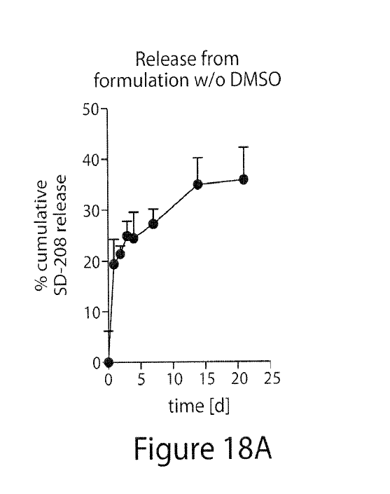

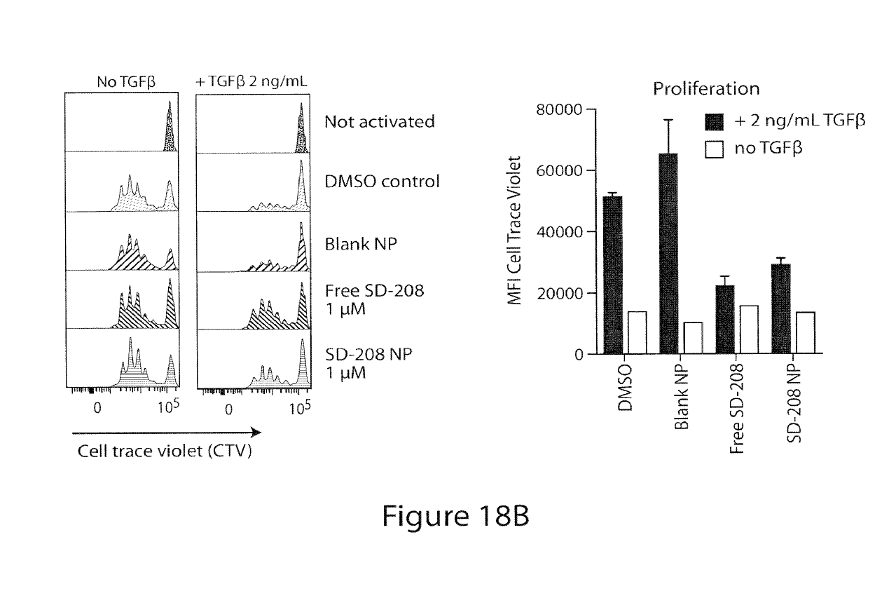

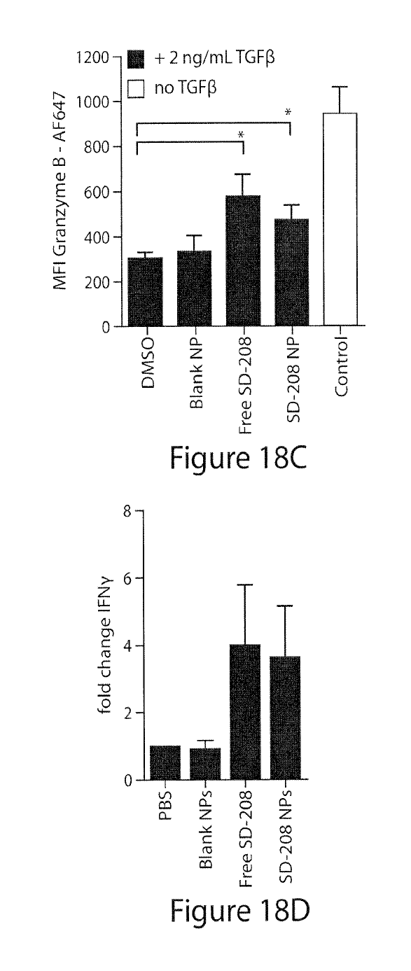

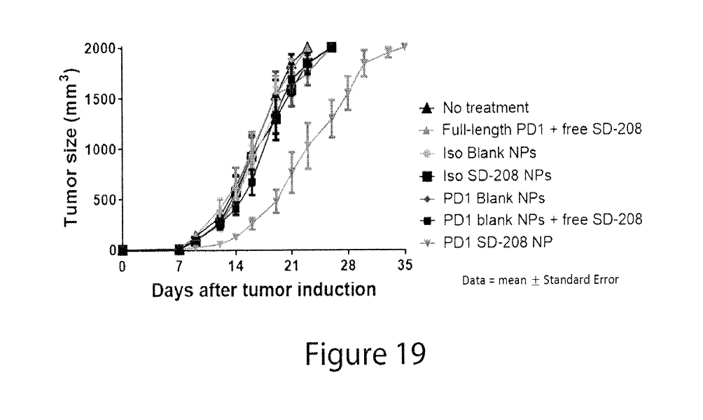

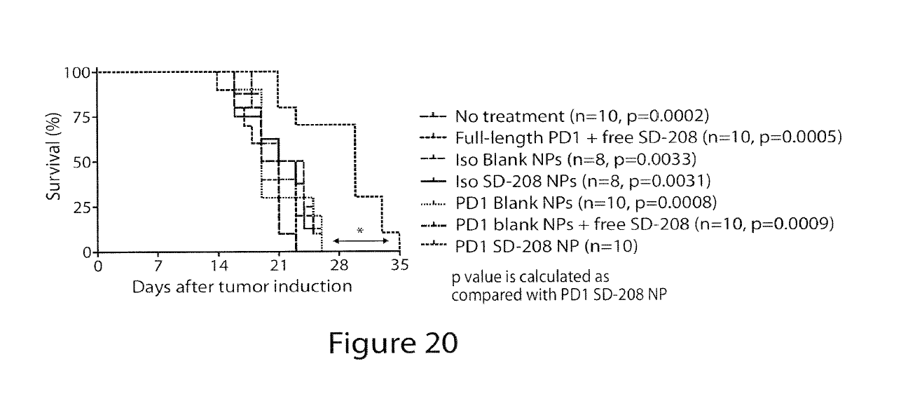

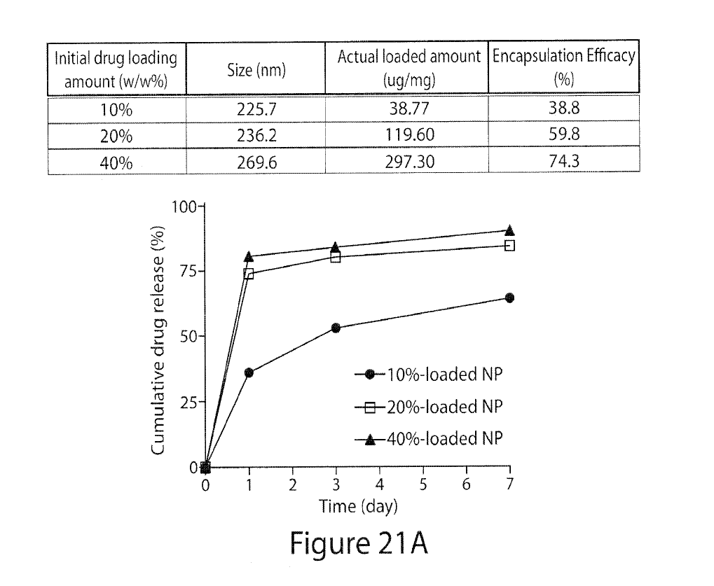

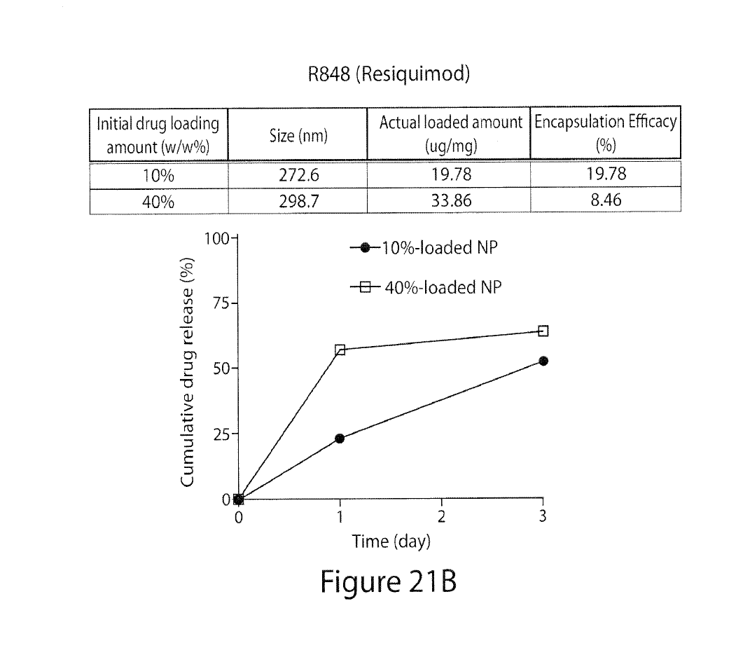

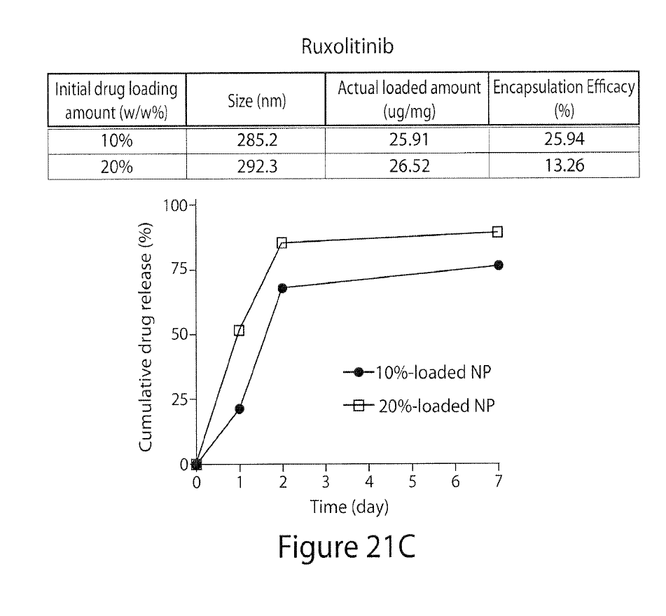

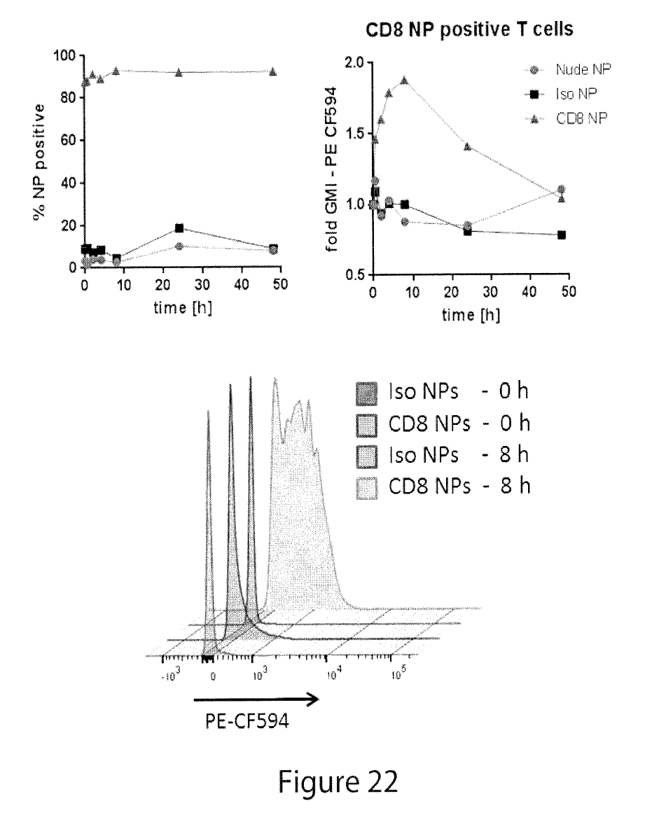

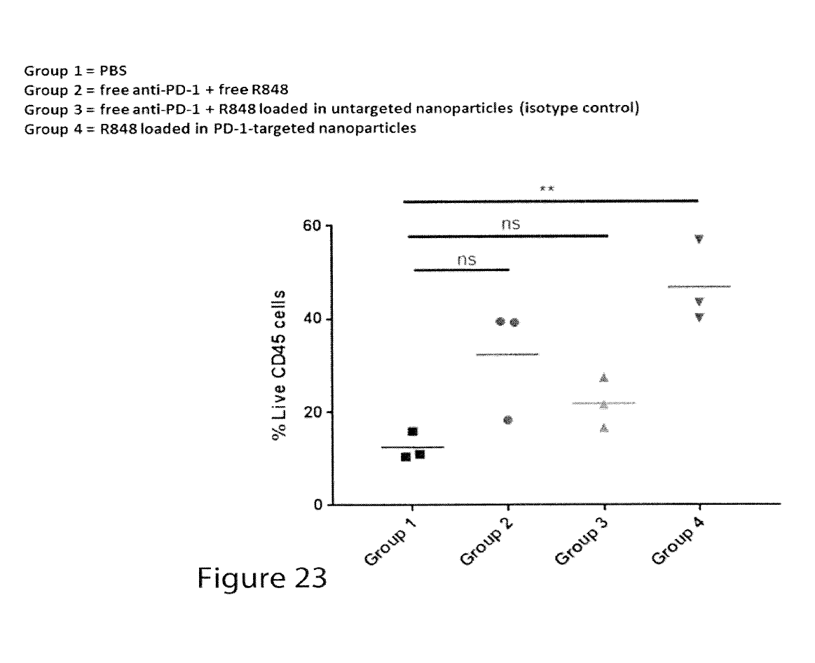

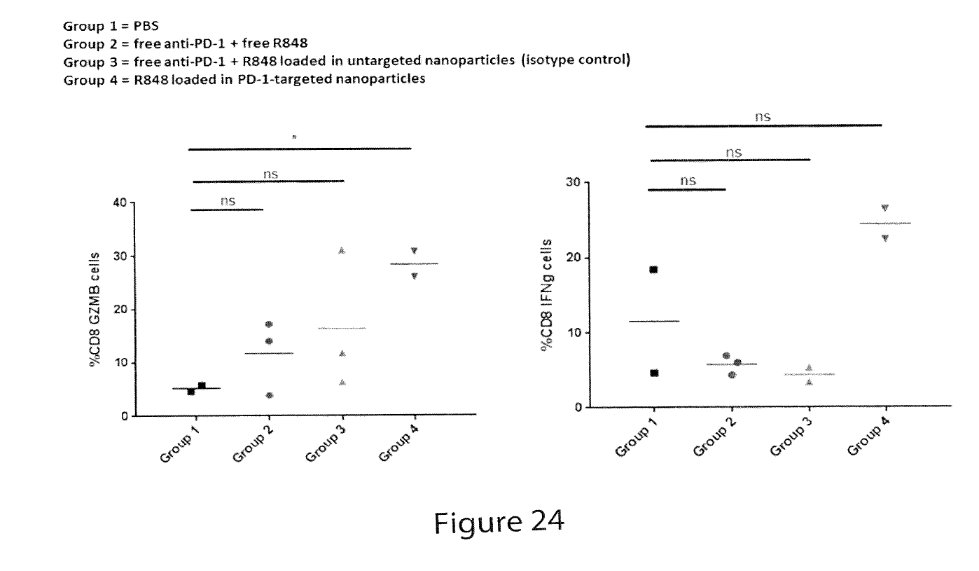

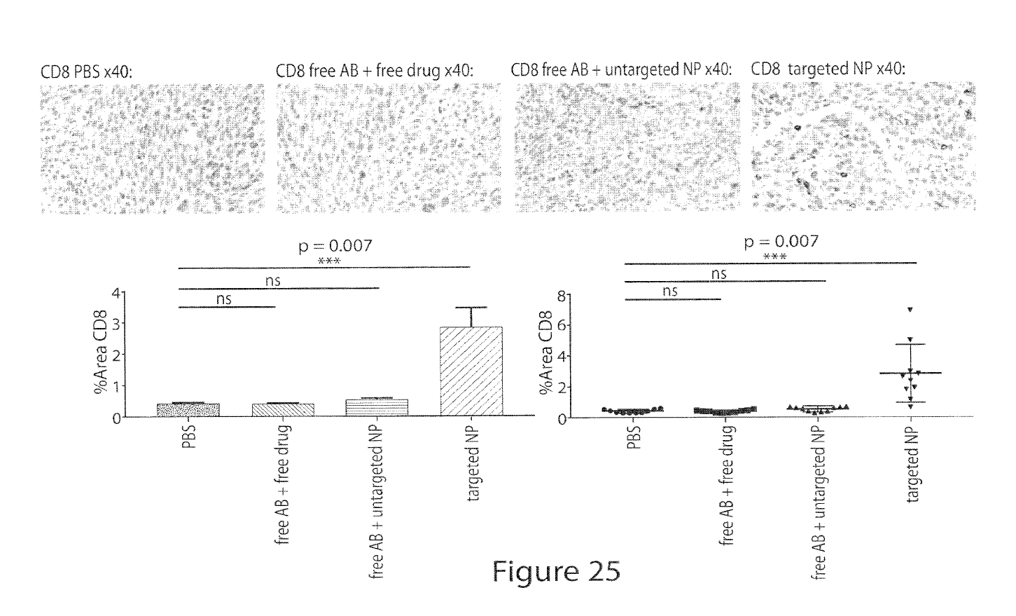

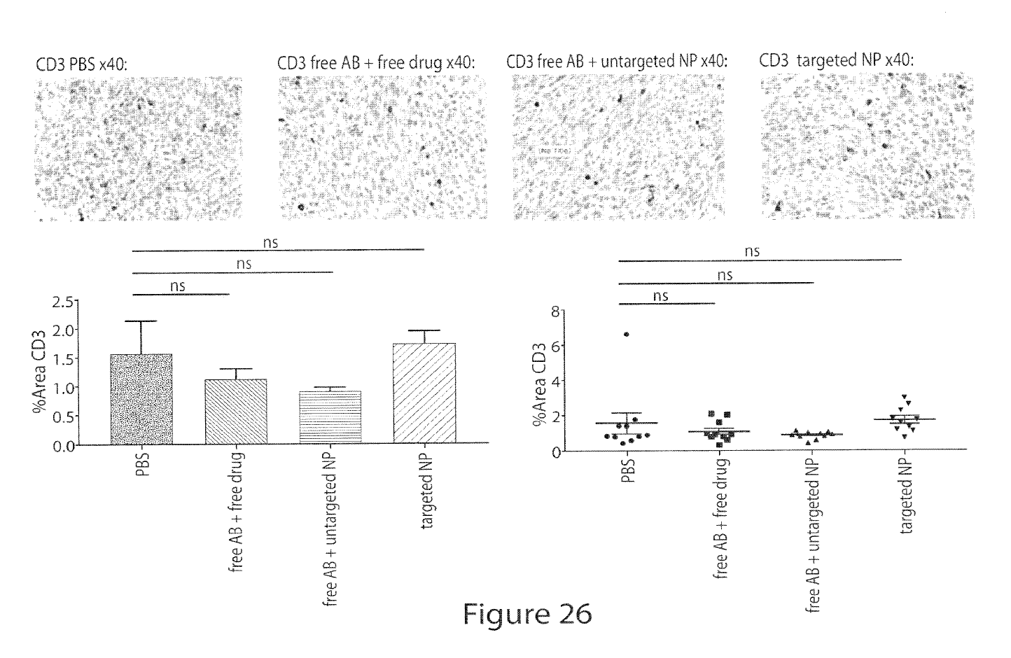

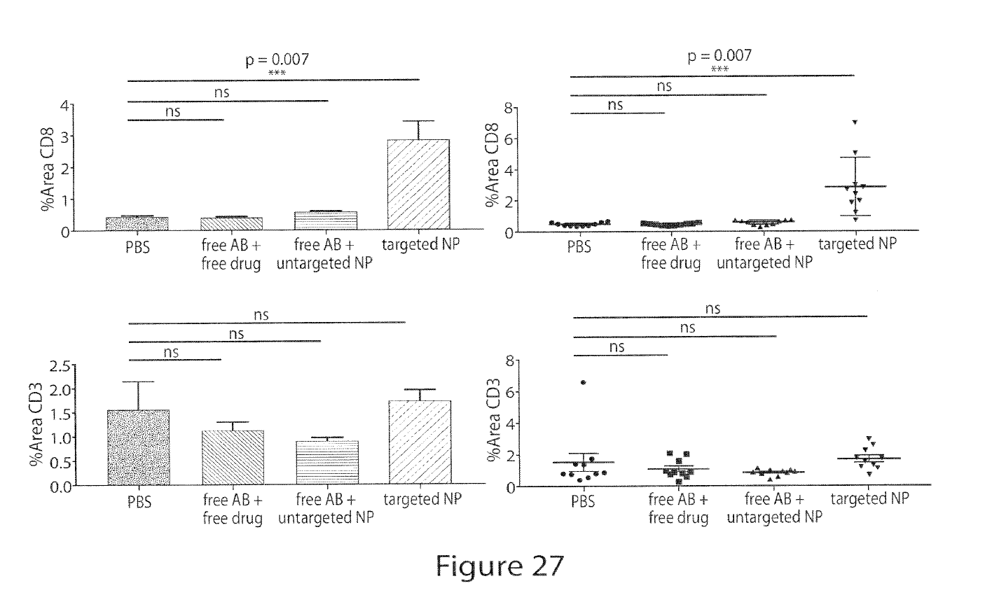

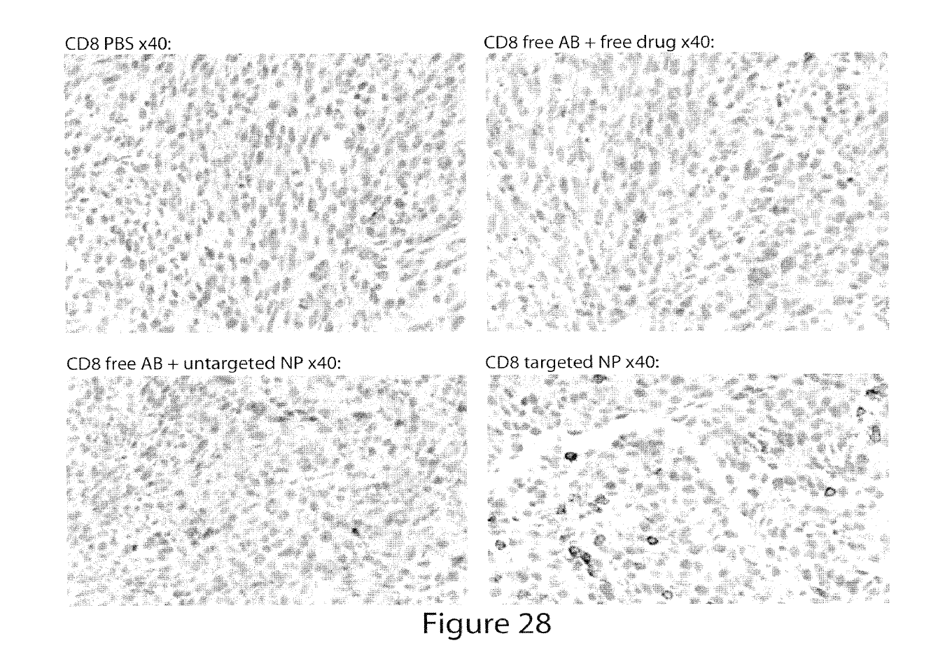

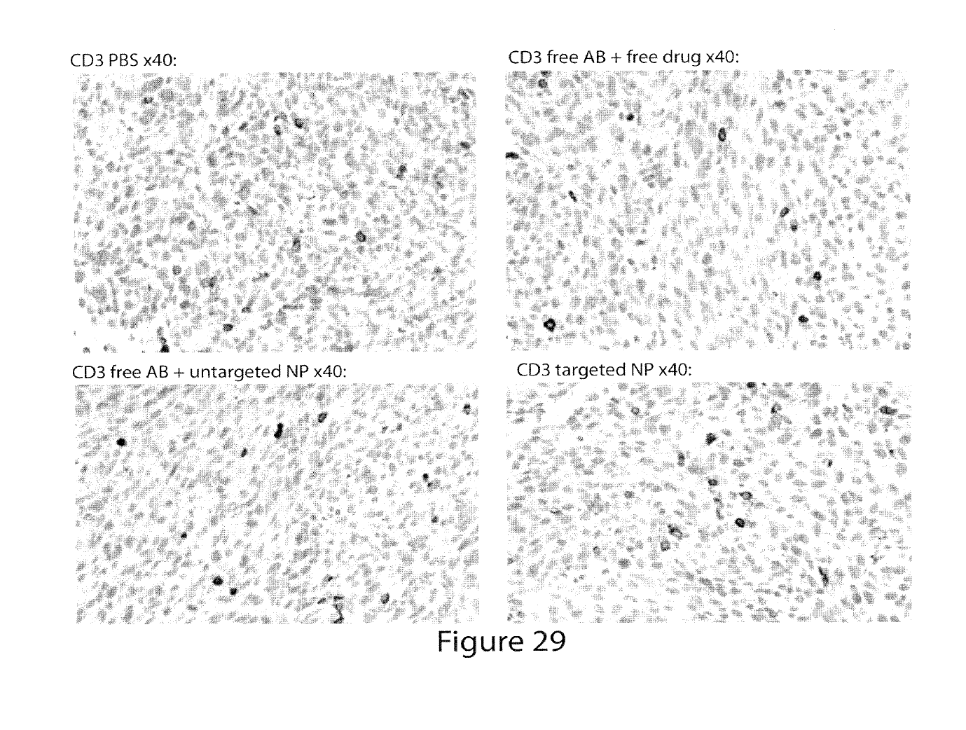

While the proof-of-concept studies were conducted by targeting CD8 as a model receptor, therapeutics studies were performed by targeting PD-1. PD-1 is an attractive receptor for targeting, as PD-1 expression defines the tumor-reactive repertoire of T cells in tumors (15) and in the circulation (16). PD-1-targeting nanoparticles accumulate in tumors more effectively than isotype control particles (FIG. 15B), suggesting that the effect may be mediated by homing of PD-1+ T cells from the blood (FIG. 16) into tumors.

Notably, the antibody fragments on the nanoparticles' surface can be used not only to target specific T cell subsets but also to functionally neutralize co-inhibitory receptors. The particles can thus both induce immune checkpoint blockade and target the sustained release of complementary small molecules to inhibit other mediators of immunosuppression in an autocrine- and/or paracrine-like manner. The platform is modular, both in terms of payload and in terms of the targeting moiety. Co-stimulatory TNF receptor superfamily members (e.g., GITR) may be of particular interest, as their natural ligands are trimeric. Monoclonal antibodies have been developed to agonize some of these targets, but the highly multivalent format afforded by the nanoparticles may add further benefit.

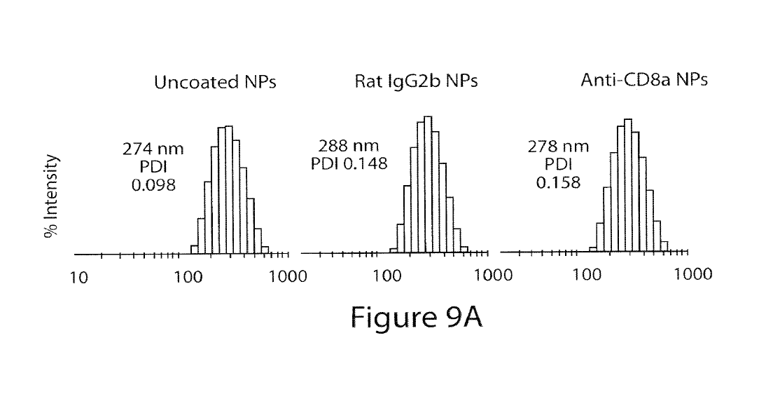

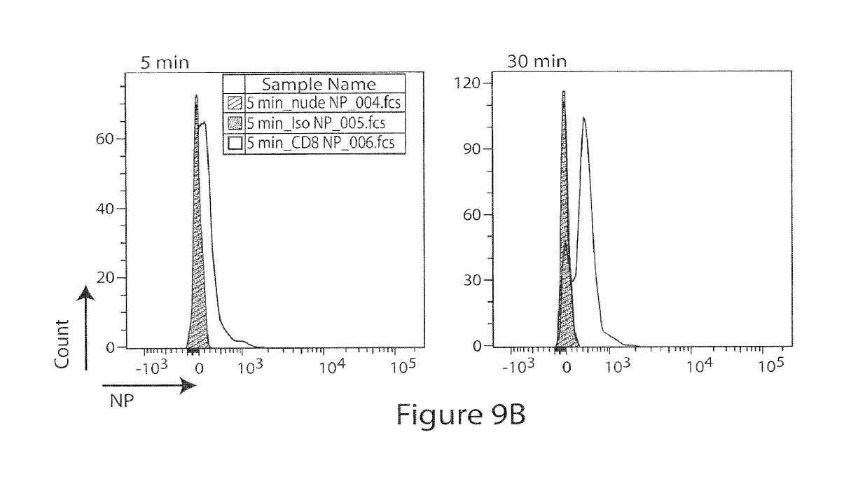

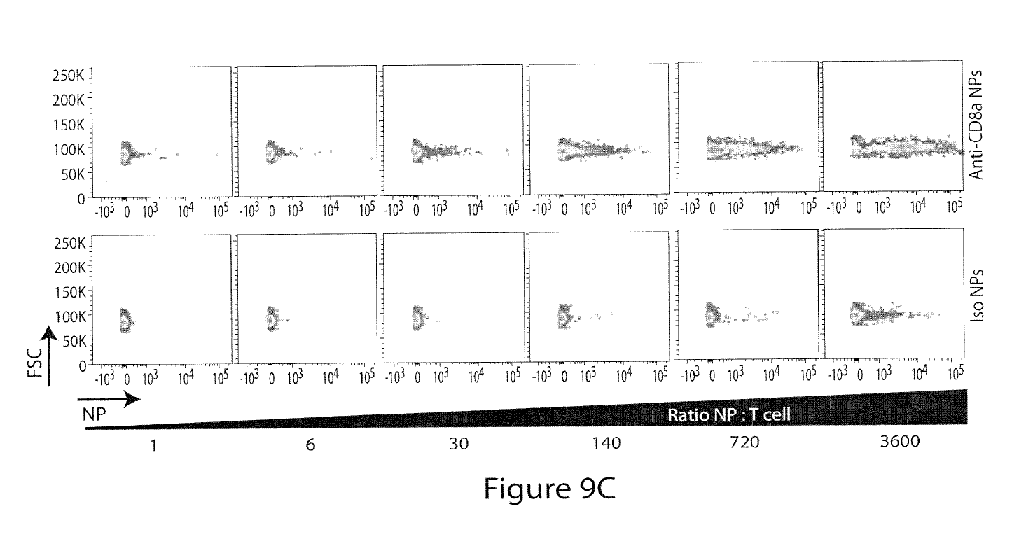

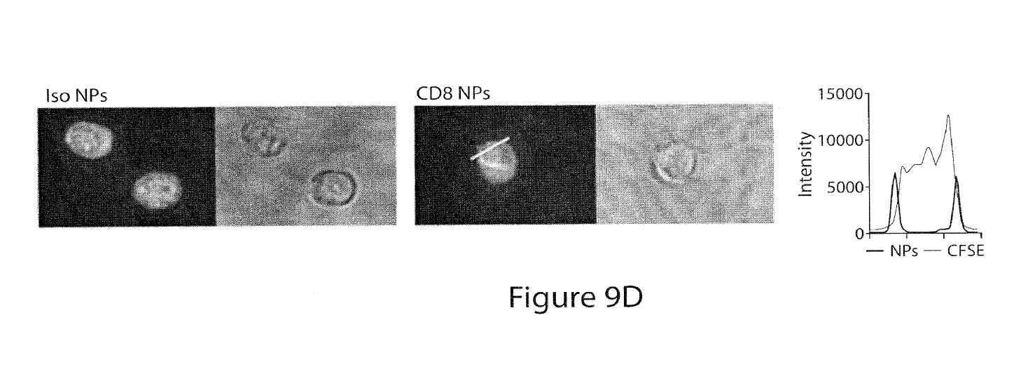

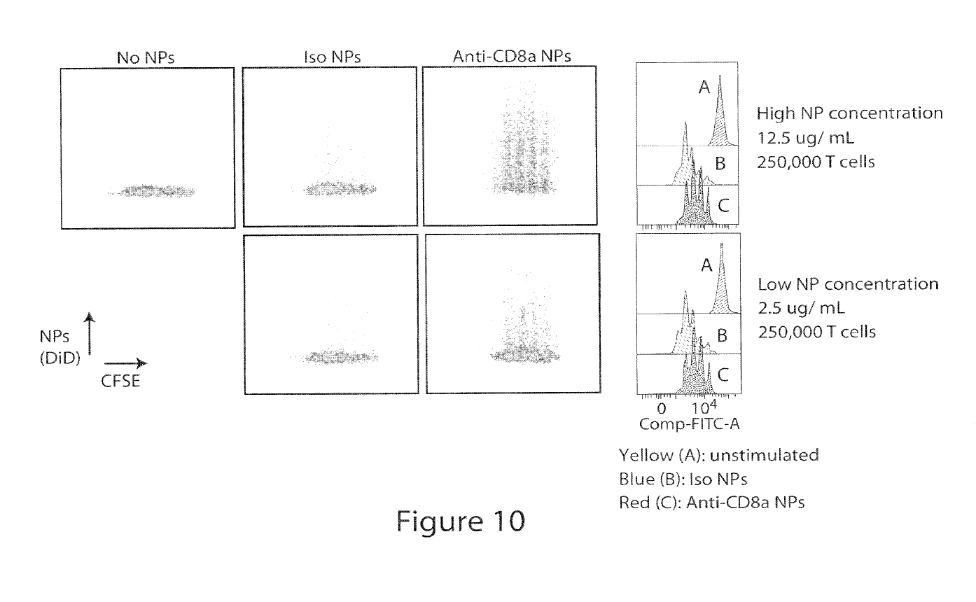

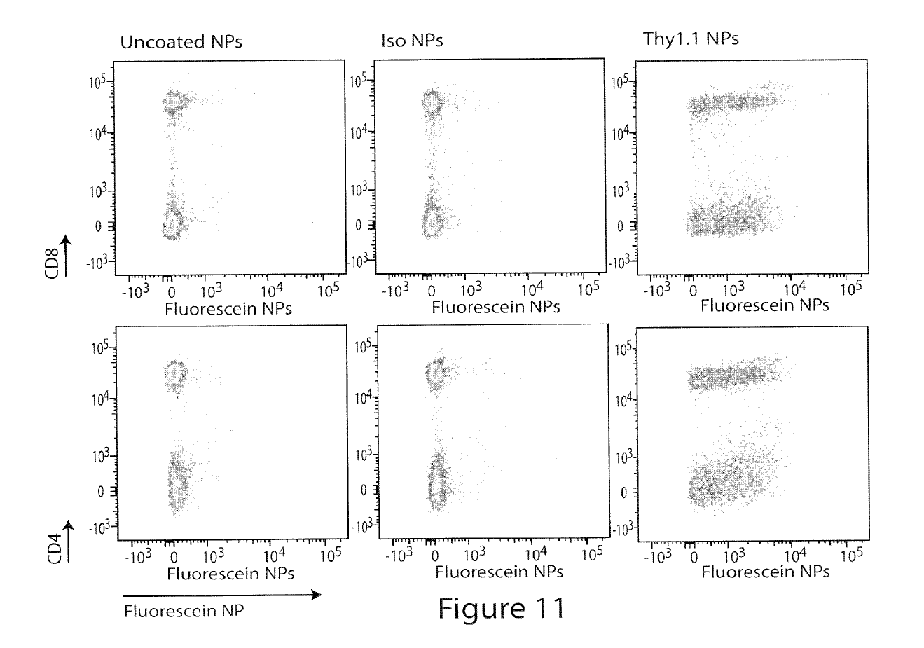

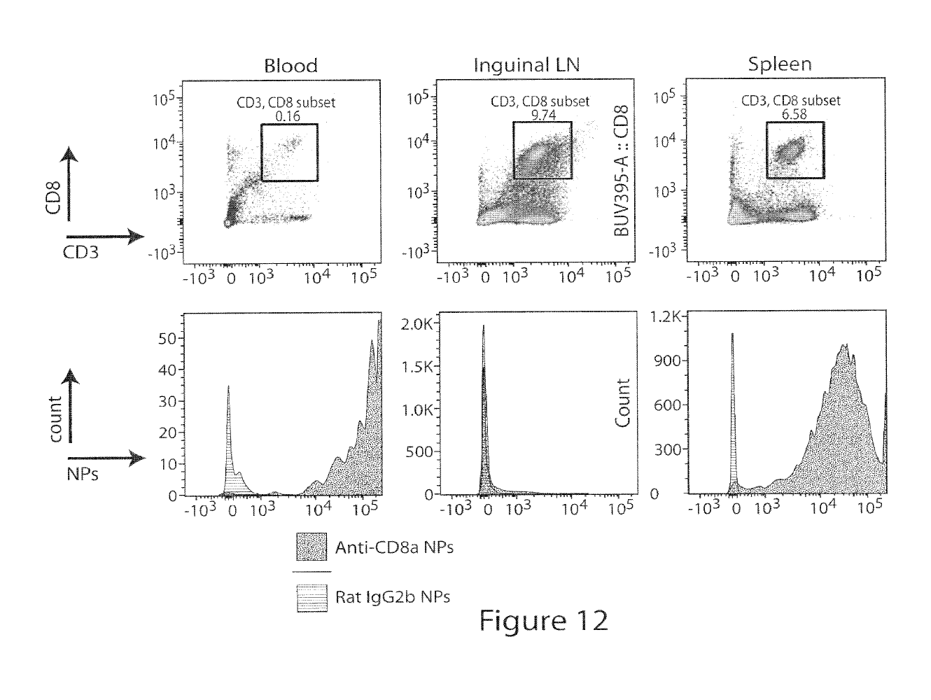

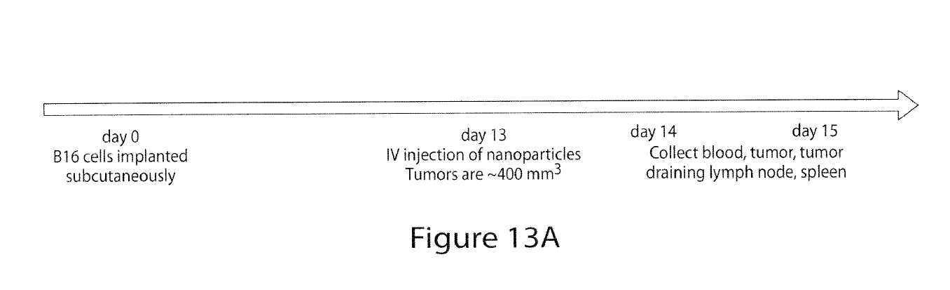

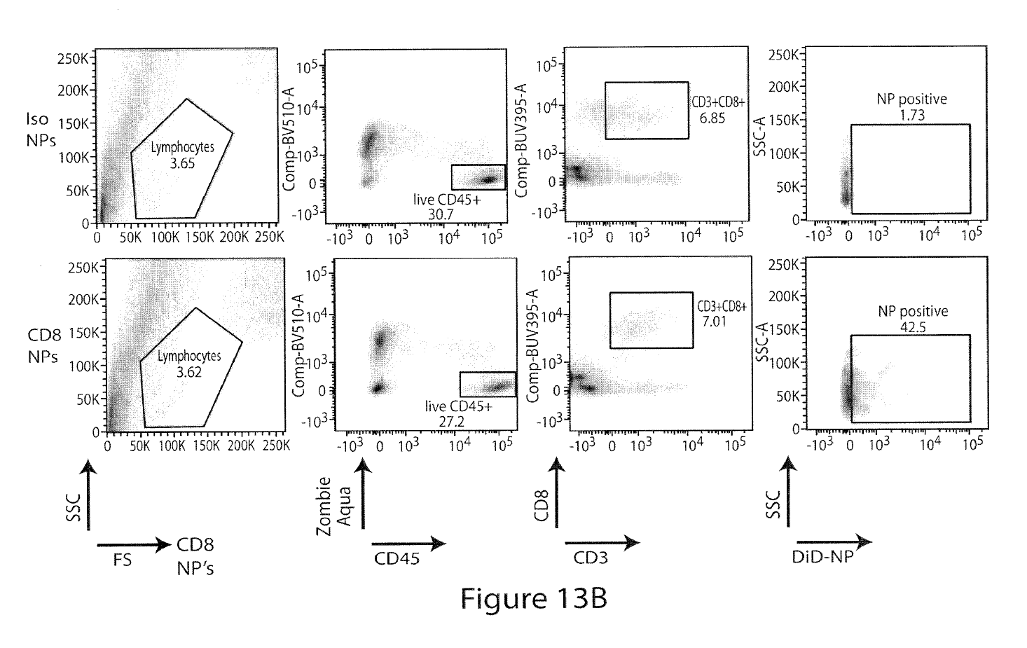

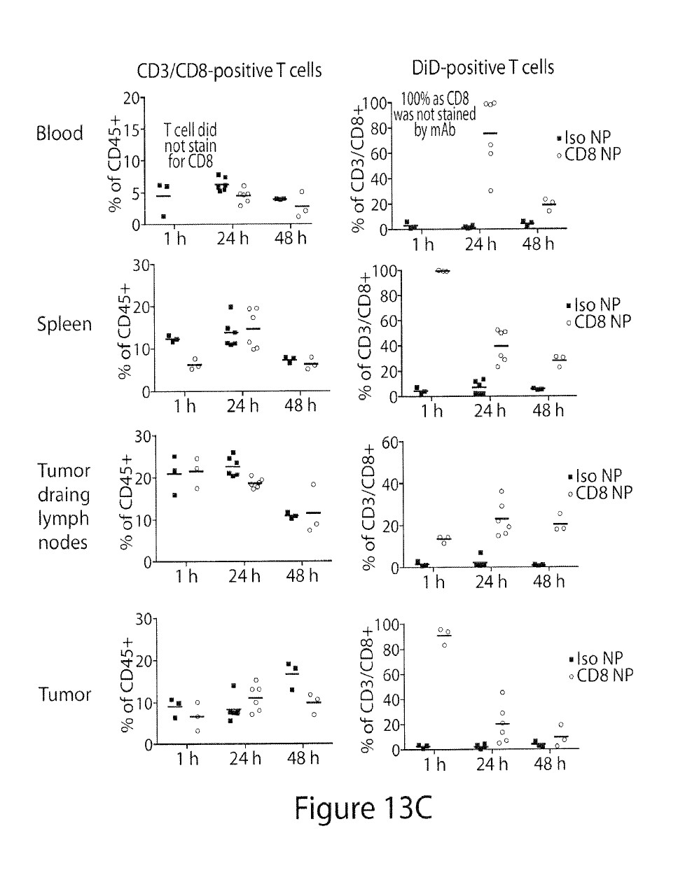

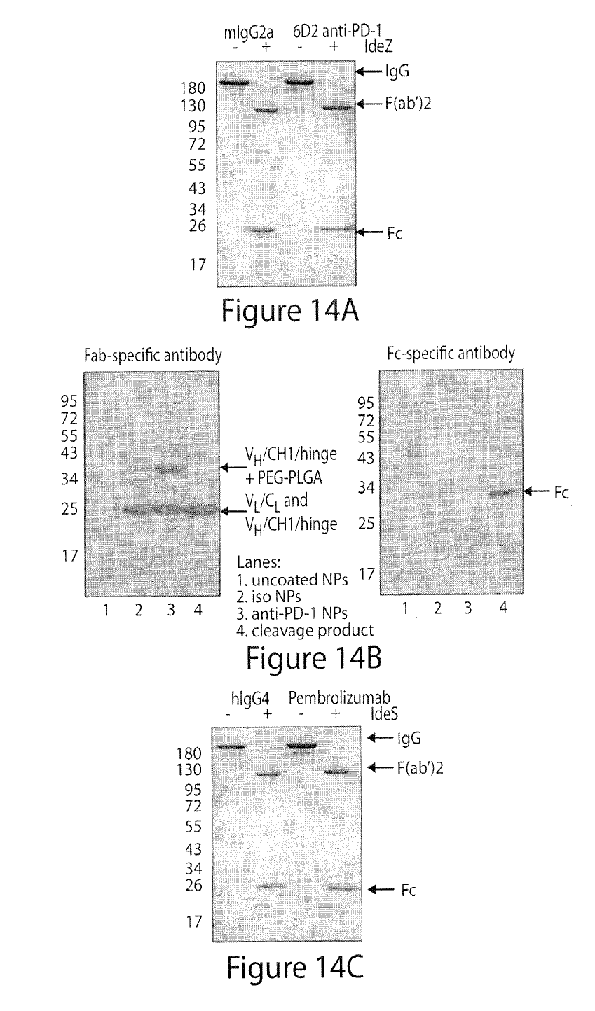

A robust in vivo T cell-targeting drug delivery system has been developed. Specific and efficient binding is observed in vitro (FIGS. 9C, 9D, 11, 31A, and 31B), including to human cells (FIGS. 33B and 33C), and in vivo (FIGS. 12, 13B, 13C, 32B, 41). Such binding allows for targeted delivery of a TGFβR1 inhibitor, delaying tumor growth and extending survival of tumor-bearing mice if and only if the inhibitor is delivered via PD-1-targeting nanoparticles (FIG. 35). Excitingly, this platform can be used to deliver immune agonists as well, which is essential to inflame tumors that are otherwise sparse for TILs. Targeted delivery of a TLR7/8 agonist, R848, promotes infiltration of CD8+ T cells into MC38 tumors, and these cells were observed to express higher levels of the antitumor effector molecules granzyme B and IFNγ (FIG. 36). Again, the effect was observed if the immunomodulatory compound was delivered via the PD-1-targeting nanoparticles, as free compounds and untargeted particles had no effect. Together, these data suggest, but are not limited to the concept, that targeting delivery of immunotherapy to endogenous immune cell subsets can improve the therapeutic index and may be worthy of additional investigation, particularly with regards to breaking immune tolerance and increasing the proportion of patients who respond to cancer immunotherapy.

### REFERENCES

1. E. J. Lipson et al., Durable cancer regression off-treatment and effective reinduction therapy with an anti-PD-1 antibody. Clin Cancer Res 19, 462-468 (2013).
2. C. F. Friedman, T. A. Proverbs-Singh, M. A. Postow, Treatment of the immune-related adverse effects of immune checkpoint inhibitors: a review. JAMA Oncol 2, 1346-1353 (2016).
3. J. Naidoo et al., Pneumonitis in patients treated with anti-Programmed Death-1/Programmed Death Ligand 1 therapy. J Clin Oncol Epub ahead of print, (2016).
4. M. Pickup, S. Novitskiy, H. L. Moses, The roles of TGFβ in the tumour microenvironment. Nat Rev Cancer 13, 788-799 (2013).
5. J. M. Yingling, K. L. Blanchard, J. S. Sawyer, Development of TGF-beta signalling inhibitors for cancer therapy. Nat Rev Drug Discov 3, 1011-1022 (2004).
6. J. Hrkach et al., Preclinical development and clinical translation of a PSMA-targeted docetaxel nanoparticle with a differentiated pharmacological profile. Sci Transl Med 4, 128ra139 (2012).
7. S. Ashton et al., Aurora kinase inhibitor nanoparticles target tumors with favorable therapeutic index in vivo. Sci Transl Med 8, 325ra317 (2016).
8. D. W. Bartlett, H. Su, I. J. Hildebrandt, W. A. Weber, M. E. Davis, Impact of tumor-specific targeting on the biodistribution and efficacy of siRNA nanoparticles measured by multimodality in vivo imaging. Proc Natl Acad Sci USA 104, 15549-15554 (2007).
9. U. Prabhakar et al., Challenges and key considerations of the enhanced permeability and retention effect for nanomedicine drug delivery in oncology. Cancer Res 73, 2412-2417 (2013).
10. B. Huang et al., Active targeting of chemotherapy to disseminated tumors using nanoparticle-carrying T cells. Sci Transl Med 7, 291ra294 (2015).
11. M. T. Stephan, J. J. Moon, S. H. Um, A. Bershteyn, D. J. Irvine, Therapeutic cell engineering with surface-conjugated synthetic nanoparticles. Nat Med 16, 1035-1041 (2010).
12. M. T. Stephan, S. B. Stephan, P. Bak, J. Chen, D. J. Irvine, Synapse-directed delivery of immunomodulators using T-cell-conjugated nanoparticles. Biomaterials 33, 5776-5787 (2012).
13. Y. Zheng et al., In vivo targeting of adoptively transferred T-cells with antibody- and cytokine-conjugated liposomes. J Control Release 172, 426-435 (2013).
14. M. Recher et al., Deliberate removal of T cell help improves virus-neutralizing antibody production. Nat Immunol 5, 934-942 (2004).
15. A. Gros et al., PD-1 identifies the patient-specific CD8+ tumor-reactive repertoire infiltrating human tumors. J Clin Invest 124, 2246-2259 (2014).
16. A. Gros et al., Prospective identification of neoantigen-specific lymphocytes in the peripheral blood of melanoma patients. Nat Med 22, 433-438 (2016).
17. A. Ribas et al., Association of Pembrolizumab with tumor response and survival among patients with advanced melanoma. JAMA 315, 1600-1609 (2016).
18. R. S. Herbst et al., Pembrolizumab versus docetaxel for previously treated, PD-L1-positive, advanced non-small-cell lung cancer (KEYNOTE-010): a randomised controlled trial. Lancet 387, 1540-1550 (2016).
19. T. Y. Seiwert et al., Safety and clinical activity of pembrolizumab for treatment of recurrent or metastatic squamous cell carcinoma of the head and neck (KEYNOTE-012): an open-label, multicentre, phase 1b trial. Lancet Oncol 17, 956-965 (2016).
20. M. Uhl et al., SD-208, a novel transforming growth factor beta receptor I kinase inhibitor, inhibits growth and invasiveness and enhances immunogenicity of murine and human glioma cells in vitro and in vivo. Cancer Res 64, 7954-7961 (2004).
21. B. Homet Moreno et al., Response to Programmed Cell Death-1 blockade in a murine melanoma syngeneic model requires costimulation, CD4, and CD8 T cells. Cancer Immunol Res 4, 845-857 (2016).
22. T. F. Gajewski, The next hurdle in cancer immunotherapy: overcoming the non-T-cell-inflamed tumor microenvironment. Semin Oncol 42, 663-671 (2015).
23. C. L. Ahonen et al., Dendritic cell maturation and subsequent enhanced T-cell stimulation induced with the novel synthetic immune response modifier R-848. Cell Immunol 197, 62-72 (1999).
24. M. Gerlinger et al., Intratumor heterogeneity and branched evolution revealed by multiregion sequencing. N Engl J Med 366, 883-892 (2012).
25. I. Mellman, G. Coukos, G. Dranoff, Cancer immunotherapy comes of age. Nature 480, 480-489 (2011).
26. M. S. Goldberg et al., Biotargeted nanomedicines for cancer: six tenets before you begin. Nanomedicine 8, 299-308 (2013).
27. Y. Shamay et al., P-selectin is a nanotherapeutic delivery target in the tumor microenvironment. Sci Transl Med 8, 345ra387 (2016).
28. J. Park et al., Modulation of CD4+ T lymphocyte lineage outcomes with targeted, nanoparticle-mediated cytokine delivery. Mol Pharm 8, 143-152 (2011).
29. M. D. McHugh et al., Paracrine co-delivery of TGF-β and IL-2 using CD4-targeted nanoparticles for induction and maintenance of regulatory T cells. Biomaterials 59, 172-181 (2015).

### Equivalents and Scope

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A particle comprising:
a polymeric core containing a pharmaceutically active agent; and
an antibody fragment conjugated to the surface of the particle, wherein the antibody fragment targets an endogenous immune cell subset.

2. The particle of claim 1, wherein the endogenous immune cell subset is a T-cell or a myeloid-derived suppressor cell.

3. (canceled)

4. The particle of claim 1, wherein the pharmaceutically active agent is a small molecule.

5-7. (canceled)

8. The particle of claim 1, wherein the pharmaceutically active agent is an immunomodulatory compound.

9. The particle of claim 8, wherein the immunomodulatory compound is a kinase inhibitor selected from the group consisting of: transforming growth factor β receptor I (TGF-βR I) kinase inhibitor, mammalian target of rapamycin (mTOR) inhibitor, glycogen synthase kinase-3β (GSK-3β) inhibitor, diacylglycerol kinase (DGK) inhibitor, proto-oncogene serine/threonine-protein kinase (PIM) inhibitor, phosphatidyl-inositol-3 kinase (PI3K) inhibitor, Janus kinase (JAK) inhibitor, mitogen-activated protein kinase (MEK) inhibitor, and combinations thereof.

10. The particle of claim 8, wherein the immunomodulatory compound that is not a kinase inhibitor is selected from the group consisting of:
indoleamine 2,3-dioxygenase (IDO1) inhibitor, tryptophan 2,3-dioxygenase (TDO2) inhibitor, arginase (ARG1) inhibitor, prostaglandin E2 (PGE2), phosphodiesterase type 5 (PDE5) inhibitor, cyclooxygenase-2 (COX2) inhibitor, inhibitors of apoptosis proteins (IAP) inhibitor, Src homology region 2 domain-containing phosphatase-1 (SHP-1) inhibitor, Src homology region 2 domain-containing phosphatase-2 (SHP-2) inhibitor, porcupine homology (PORCN) inhibitor, adenosine A2A receptor (A2AR) inhibitor, colony-stimulating factor 1 receptor (CSF1R) inhibitor, macrophage-stimulating protein receptor (RON) inhibitor, and combinations thereof.

11. The particle of claim 8, wherein the immunomodulatory compound is a an agonist of a Toll-like receptor (TLR), a C-type lectin receptor (CLR), or a NOD-like receptor (NLR) selected from the group consisting of: TLR2 agonist, TLR4 agonist, TLR5 agonist, TLR7 agonist, TLR8 agonist, Dectin-1 agonist, Dectin-2 agonist, Mincle agonist, NOD1 agonist, NOD2 agonist, and combinations thereof.

12-16. (canceled)

17. The particle of claim 11, wherein the immunomodulatory compound increases the proportion of CD8+ T cells in a tumor.

18-20. (canceled)

21. The particle of claim 1, wherein the antibody fragment is a F(ab′)2 fragment, Fab fragment, or Fab′ fragment.

22-23. (canceled)

24. The particle of claim 1, wherein the antibody fragment targets endogenous T-cells.

25. (canceled)

26. The particle of claim 1, wherein the antibody fragment targets a marker expressed on the surface of myeloid-derived suppressor cells.

27-37. (canceled)

38. The particle of claim 1, wherein the antibody fragment comprises two antibodies, wherein one antibody targets CD8, and a second antibody targets PD-1.

39. The particle of claim 1, wherein the particle comprises two antibodies, wherein one antibody targets PD-1, and a second antibody targets GITR.

40. The particle of claim 1, wherein the particle comprises two antibodies, wherein one antibody targets PD-1, and a second antibody targets LAG-3 or TIM-3.

41. The particle of claim 1, wherein the antibody fragment targets a peripheral T-cell or a tumor-resident T-cell.

42. The particle of claim 1, wherein the antibody fragment targets an activated T-cell.

43-47. (canceled)

48. The particle of claim 1, wherein the particle comprises a corona around at least a portion of the surface of the particle core.

49-63. (canceled)

64. A pharmaceutical composition comprising:
a plurality of particles of claim 1; and
a pharmaceutically acceptable excipient.

65. (canceled)

66. A method of treating a proliferative disease in a subject comprising:
administering the particle of claim 1.

67-74. (canceled)

75. A method of forming a particle comprising:
providing a polymeric core containing a pharmaceutically active agent; and
conjugating an antibody fragment to the surface of the particle, wherein the antibody fragment targets an endogenous immune cell subset, to form a particle as in claim 1.

76-84. (canceled)
